SEQUENCE LISTING (31 sequences)

SEQ ID NO: 1 - protein, 184 aa, synthetic construct
KYNNAKVTVDTVCKRGFLIQMSGHLECKCENDLVLVNEETCEEKVLKCDEKTVNKPCGDFSKCIKIDGNPVSYACKCNLGYDMVNNVCIPNECKNVTCGNGKCILDTSNPVKTGVCSCNIGKVPNVQDQNKCSKDGETKCSLKCLKENETCKAVDGIYKCDCKDGFIIDNESSICTAFSAYNIL

SEQ ID NO: 2 - protein, 355 aa, synthetic construct
QEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCS

SEQ ID NO: 3 - protein, 17 aa, synthetic construct
MNKLYSLFLFLFIQLSI

SEQ ID NO: 4 - protein, 20 aa, synthetic construct
MMRKLAILSVSSFLFVEALF

SEQ ID NO: 5 - protein, 16 aa, synthetic construct
NLSIMFILFSVCFFIM

SEQ ID NO: 6 - protein, 22 aa, synthetic construct
SVFNVVNSSIGLIMVLSFLFLN

SEQ ID NO: 7 - protein, 217 aa, synthetic construct
MNKLYSLFLFLFIQLSIKYNNAKVTVDTVCKRGFLIQMSGHLECKCENDLVLVNEETCEEKVLKCDEKTVNKPCGDFSKCIKIDGNPVSYACKCNLGYDMVNNVCIPNECKNVTCGNGKCILDTSNPVKTGVCSCNIGKVPNVQDQNKCSKDGETKCSLKCLKENETCKAVDGIYKCDCKDGFIIDNESSICTAFSAYNILNLSIMFILFSVCFFIM

SEQ ID NO: 8 - protein, 397 aa, synthetic construct
MMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLN

SEQ ID NO: 9 - DNA, 654 bp, synthetic construct
  source 1..654: mol_type = other DNA
atgaataaactttacagtttgtttcttttccttttcattcaacttagcataaaatataataatgcgaaagttaccgtggatactgtatgcaaaagaggatttttaattcagatgagtggtcatttggaatgtaaatgtgaaaatgatttggtgttagtaaatgaagaaacatgtgaagaaaaagttctgaaatgtgacgaaaagactgtaaataaaccatgtggagatttttccaaatgtattaaaatagatggaaatcccgtttcatacgcttgtaaatgtaatcttggatatgatatggtaaataatgtttgtataccaaatgaatgtaagaatgtaacttgtggtaacggtaaatgtatattagatacaagcaatcctgttaaaactgcagtttgctcatgtaatataggcaaagttcccaatgtacaagatcaaaataaatgttcaaaagatggagaaaccaaatgctcattaaaatgcttaaaagaaaatgaaacctgtaaagctgttgatggaatttataaatgtgattgtaaagatggatttataatagataatgaaagctctatatgtactgctttttcagcatataatattttaaatctaagcattatgtttatactattttcagtatgcttttttataatgtaa

SEQ ID NO: 10 - DNA, 1194 bp, synthetic construct
  source 1..1194: mol_type = other DNA
atgatgagaaaattagctattttatctgtttcttcctttttatttgttgaggccttattccaggaataccagtgctatggaagttcgtcaaacacaagggttctaaatgaattaaattatgataatgcaggcactaatttatataatgaattagaaatgaattattatgggaaacaggaaaattggtatagtcttaaaaaaaatagtagatcacttggagaaaatgatgatggaaataacgaagacaacgagaaattaaggaaaccaaaacataaaaaattaaagcaaccagcggatggtaatcctgatccaaatgcaaacccaaatgtagatcccaatgccaacccaaatgtagatccaaatgcaaacccaaatgtagatccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaaccccaatgcaaatcctaatgcaaacccaaatgcaaacccaaacgtagatcctaatgcaaatccaaatgcaaacccaaacgcaaaccccaatgcaaatcctaatgcaaaccccaatgcaaatcctaatgcaaatcctaatgccaatccaaatgcaaatccaaatgcaaacccaaacgcaaaccccaatgcaaatcctaatgccaatccaaatgcaaatccaaatgcaaacccaaatgcaaacccaaatgcaaaccccaatgcaaatcctaataaaaacaatcaaggtaatggacaaggtcacaatatgccaaatgacccaaaccgaaatgtagatgaaaatgctaatgccaacagtgctgtaaaaaataataataacgaagaaccaagtgataagcacataaaagaatatttaaacaaaatacaaaattctctttcaactgaatggtccccatgtagtgtaacttgtggaaatggtattcaagttagaataaagcctggctctgctaataaacctaaagacgaattagattatgcaaatgatattgaaaaaaaaatttgtaaaatggaaaaatgttccagtgtgtttaatgtcgtaaatagttcaataggattaataatggtattatccttcttgttccttaattag

SEQ ID NO: 11 - DNA, 6239 bp, synthetic construct
  source 1..6239: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcgtcgacagagctgagatcctacaggagtccagggctggagagaaaacctctgcgaggaaaaggaaggagcaagccgtgaatttaagggacgctgtgaagcaatcatggatgcaatgaagagagggctctgctgtgtgctgctgctgtgtggagcagtcttcgtttcgcccagcggtaccggatccatgatgagaaaattagctattttatctgtttcttcctttttatttgttgaggccttattccaggaataccagtgctatggaagttcgtcaaacacaagggttctaaatgaattaaattatgataatgcaggcactaatttatataatgaattagaaatgaattattatgggaaacaggaaaattggtatagtcttaaaaaaaatagtagatcacttggagaaaatgatgatggaaataacgaagacaacgagaaattaaggaaaccaaaacataaaaaattaaagcaaccagcggatggtaatcctgatccaaatgcaaacccaaatgtagatcccaatgccaacccaaatgtagatccaaatgcaaacccaaatgtagatccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaaccccaatgcaaatcctaatgcaaacccaaatgcaaacccaaacgtagatcctaatgcaaatccaaatgcaaacccaaacgcaaaccccaatgcaaatcctaatgcaaaccccaatgcaaatcctaatgcaaatcctaatgccaatccaaatgcaaatccaaatgcaaacccaaacgcaaaccccaatgcaaatcctaatgccaatccaaatgcaaatccaaatgcaaacccaaatgcaaacccaaatgcaaaccccaatgcaaatcctaataaaaacaatcaaggtaatggacaaggtcacaatatgccaaatgacccaaaccgaaatgtagatgaaaatgctaatgccaacagtgctgtaaaaaataataataacgaagaaccaagtgataagcacataaaagaatatttaaacaaaatacaaaattctctttcaactgaatggtccccatgtagtgtaacttgtggaaatggtattcaagttagaataaagcctggctctgctaataaacctaaagacgaattagattatgcaaatgatattgaaaaaaaaatttgtaaaatggaaaaatgttccagtgtgtttaatgtcgtaaatagttcaataggattaataatggtattatccttcttgttccttaattagagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 12 - DNA, 5625 bp, synthetic construct
  source 1..5625: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcgtcgacagagctgagatcctacaggagtccagggctggagagaaaacctctgcgaggaaaaggaaggagcaagccgtgaatttaagggacgctgtgaagcaatcatggatgcaatgaagagagggctctgctgtgtgctgctgctgtgtggagcagtcttcgtttcgcccagcggtaccggatccaagtacaacaacgccaaggtgaccgtggacaccgtgtgcaagcgcggcttcctgatccagatgagcggccacctggagtgcaagtgcgagaacgacctggtgctggtgaacgaggagacctgcgaggagaaggtgctgaagtgcgacgagaagaccgtgaacaagccctgcggcgacttcagcaagtgcatcaagatcgacggcaaccccgtgagctacgcctgcaagtgcaacctgggctacgacatggtgaacaacgtgtgcatccccaacgagtgcaagaacgtgacctgcggcaacggcaagtgcatcctggacaccagcaaccccgtgaagaccggcgtgtgcagctgcaacatcggcaaggtgcccaacgtgcaggaccagaacaagtgcagcaaggacggcgagaccaagtgcagcctgaagtgcctgaaggagaacgagacctgcaaggccgtggacggcatctacaagtgcgactgcaaggacggcttcatcatcgacaacgagagcagcatctgcaccgccttcagcgcctacaacatcctgtaaaagcttagaacctgaattcggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 13 - protein, 202 aa, synthetic construct
HMAHHHHHHPGGSGSGTKYNNAKVTVDTVCKRGFLIQMSGHLECKCENDLVLVNEETCEEKVLKCDEKTVNKPCGDFSKCIKIDGNPVSYACKCNLGYDMVNNVCIPNECKNVTCGNGKCILDTSNPVKTGVCSCNIGKVPNVQDQNKCSKDGETKCSLKCLKENETCKAVDGIYKCDCKDGFIIDNESSICTAFSAYNILN

SEQ ID NO: 14 - protein, 20 aa, synthetic construct
KYNNAKVTVDTVCKRGFLIQ

SEQ ID NO: 15 - protein, 20 aa, synthetic construct
TVCKRGFLIQMSGHLECKCE

SEQ ID NO: 16 - protein, 20 aa, synthetic construct
MSGHLECKCENDLVLVNEET

SEQ ID NO: 17 - protein, 20 aa, synthetic construct
NDLVLVNEETCEEKVLKCDE

SEQ ID NO: 18 - protein, 20 aa, synthetic construct
CEEKVLKCDEKTVNKPCGDF

SEQ ID NO: 19 - protein, 20 aa, synthetic construct
KTVNKPCGDFSKCIKIDGNP

SEQ ID NO: 20 - protein, 20 aa, synthetic construct
SKCIKIDGNPVSYACKCNLG

SEQ ID NO: 21 - protein, 20 aa, synthetic construct
VSYACKCNLGYDMVNNVCIP

SEQ ID NO: 22 - protein, 20 aa, synthetic construct
YDMVNNVCIPNECKNVTCGN

SEQ ID NO: 23 - protein, 20 aa, synthetic construct
NECKNVTCGNGKCILDTSNP

SEQ ID NO: 24 - protein, 20 aa, synthetic construct
GKCILDTSNPVKTGVCSCNI

SEQ ID NO: 25 - protein, 20 aa, synthetic construct
VKTGVCSCNIGKVPNVQDQN

SEQ ID NO: 26 - protein, 20 aa, synthetic construct
GKVPNVQDQNKCSKDGETKC

SEQ ID NO: 27 - protein, 20 aa, synthetic construct
KCSKDGETKCSLKCLKENET

SEQ ID NO: 28 - protein, 20 aa, synthetic construct
SLKCLKENETCKAVDGIYKC

SEQ ID NO: 29 - protein, 20 aa, synthetic construct
CKAVDGIYKCDCKDGFIIDN

SEQ ID NO: 30 - protein, 24 aa, synthetic construct
DCKDGFIIDNESSICTAFSAYNIL

SEQ ID NO: 31 - protein, 8 aa, synthetic construct
PGGSGSGT

(19) United States
(12) Patent Application Publication
(10) Pub. No.: US 2024/0343764 A1
(43) Pub. Date: Oct. 17, 2024

(54) COMPOSITIONS FOR AND METHODS OF TREATING AND/OR PREVENTING MALARIA

(71) Applicants: THE GEORGE WASHINGTON UNIVERSITY, Washington, DC (US); THE TRUSTEES OF THE UNIVERSITY OF PENNSYLVANIA, Philadelphia, PA (US)

(72) Inventors: NIRBHAY KUMAR, Washington, DC (US); CLIFFORD THOMAS HOLESKI HAYASHI, Washington, DC (US); YI CAO, Washington, DC (US); DREW WEISSMAN, Philadelphia, PA (US)

(21) Appl. No.: 18/661,538

(22) Filed: May 10, 2024

Related U.S. Application Data
(63) Continuation of application No. PCT/US22/80148, filed on Nov. 18, 2022.
(60) Provisional application No. 63/264,288, filed on Nov. 18, 2021.

(51) Int. Cl. C07K 14/445; A61K 39/00; A61P 33/06

(52) U.S. Cl. CPC C07K 14/445; A61P 33/06; A61K 2039/53; A61K 2039/55555

(57) ABSTRACT

Disclosed herein are methods inducing an antigen specific immune response, methods of interrupting malaria transmission, and methods of treating and/or preventing malaria. Disclosed herein are mRNA molecules, viral vectors, mRNA vaccines, and pharmaceutical formulations for use in the disclosed methods.

### CROSS-REFERENCE TO RELATED APPLICATIONS

This application is a continuation of PCT Patent Application No. PCT/US22/80148, filed 18 Nov. 2022, which claims the benefit of priority to U.S. Provisional Patent Application No. 63/264,288 filed 18 Nov. 2021, each of which is incorporated by reference herein in its entirety.

### STATEMENT REGARDING FEDERALLY SPONSORED RESEARCH OR DEVELOPMENT

This invention was made with government support under RO1 AI127544 awarded by the National Institutes of Health. The government has certain rights in the invention.

### REFERENCE TO THE SEQUENCE LISTING

The Sequence Listing submitted 18 Nov. 2022 as an XML file named “091019_709889_022_008_Kumar”, created on 15 Nov. 2022 and having a size of 46 kilobytes is hereby incorporated by reference pursuant to 37 C.F.R. § 1.52 (e) (5).

### BACKGROUND

Malaria is a deadly disease responsible for between 550,000 and 627,000 deaths annually. Over the past few decades, progress has been made toward reducing malaria incidence through mosquito control interventions and increased access to antimalarial drugs. Unfortunately, drug resistance towards frontline antimalarial drugs continues to increase, and overall progress in incidence reduction has started to stagnate.

Thus, there remains an urgent and unmet medical need for developing effective and safe vaccines that target multiple stages of the complex life cycle of the parasite responsible for malaria.

### BRIEF DESCRIPTION OF THE FIGURES

FIG. 1A-FIG. 1B show the timelines for mRNA immunization experiment FIG. 1A shows the groups of female Balb/c mice were immunized (Im) with 3 μg, 10 μg, and 30 μg of mRNA-LNPs encoding either Pfs25 or PfCSP. Another group of mice received a combination of 10 μg of both Pfs25 and PfCSP mRNA-LNPs. In parallel, mice were also immunized with 50 μg of DNA plasmids encoding Pfs25 or PfCSP and a combination of 25 μg of both Pfs25 and PfCSP DNA plasmids. Immunizations were repeated as indicated, and test bleeds (B1-B4) and final bleeds (FB1 and FB2) were collected at indicated time points. Mice immunized with 3 μg and 10 μg Pfs25 mRNA-LNP were terminally bled at 12 weeks (FB1) while all other groups were terminally bled at 24 weeks (FB2). Mice immunized with PfCSP mRNA or DNA plasmids were challenged (Ch1 and Ch2) with sporozoites of PbPfCSP-GFPLuc to assess protection. 30 μg Pfs25 mRNA-LNP and Pfs25 DNA immunized mice were used as a negative control for sporozoite challenge experiments. FIG. 1B provides a table of immunization parameters corresponding to timeline in FIG. 1A.

FIG. 2A-FIG. 2D show the experimental details for immunization experiment to evaluate lower vaccine doses and repeat immunizations. Groups of female Balb/c mice were immunized with 10 μg of either Pfs25 or PfCSP mRNA LNPs followed by a challenge with sporozoites after one (FIG. 2A), two (FIG. 2B), and three and four immunizations (FIG. 2C) as indicated. Two additional groups of mice for each mRNA-LNP were immunized with lower doses (0.1 μg and 1 μg) mRNA-LNPs to assess minimum immunogenic doses of Pfs25 or PfCSP mRNA-LNPs. B3 collected at 11 weeks from mice immunized with 0.1 μg, 1.0 μg, and 10.0 μg doses of Pfs25 mRNA-LNP were used in SMFA. Bleeds FB1 (FIG. 2A), FB2 (FIG. 2B), and B4 (FIG. 2C) were used in SMFA to evaluate transmission blocking activity in mice receiving one, two, or three immunizations, respectively. FIG. 2D shows the immunization parameters corresponding to the timelines in FIG. 2A, FIG. 2B, and FIG. 2C.

FIG. 3A-FIG. 3D show Pfs25-specific antibody titers determined by ELISA. mRNA-LNP immunization and blood collection schedules are shown in FIG. 1A. Sera (n=5 per group) from bleeds at indicated time points [B1 in FIG. 3A, B2 in FIG. 3B, B3 in FIG. 3C, and B4 in FIG. 3D] were analyzed using ELISA to determine antigen-specific antibody titers reported as the geometric mean endpoint titers with 95% confidence intervals. Endpoint titers were identified as the highest reciprocal serum dilution with an OD higher than a cutoff of the average OD plus three standard deviations of pre-immune sera replicates. Statistical analysis was performed using the Mann-Whitney U test (*p<0.05; **p<0.01).

FIG. 4A-FIG. 4D show PfCSP-specific antibody titers determined by ELISA. mRNA-LNP immunization and blood collection schedules are shown in FIG. 4A. Sera from bleeds at indicated time points [B1 in panel FIG. 4A, B2 in panel FIG. 4B, B3 in panel FIG. 4C, and B4 in panel FIG. 4D] were analyzed using ELISA to determine antigen-specific antibody titers reported as the geometric mean endpoint titers with 95% confidence intervals. Endpoint titers were identified as the highest reciprocal serum dilution with an OD higher than a cutoff of the average OD plus three standard deviations of pre-immune sera replicates. Statistical analysis was performed using the Mann-Whitney U test (*p<0.05)

FIG. 5 show evaluation of transmission reducing activity of antibodies induced by Pfs25 mRNA-LNPs. IgG was purified from pooled sera collected from mice immunized with 3 μg and 10 μg of Pfs25 mRNA-LNP (B3) and 30 μg Pfs25 mRNA and 10 μg of both Pfs25+PfCSP mRNA-LNP combinations groups (B4). Purified IgG were evaluated in mosquito standard membrane feeding assays (SMFA) at indicated final concentrations (0.25 mg/mL, 0.125 mg/mL, 0.63 mg/mL, and 0.031 mg/mL). Data points representing the oocyst counts from each mosquito, arithmetic means, and 95% confidence intervals are reported. N is the fraction of uninfected mosquitoes over the total number of mosquitoes dissected. % TRA is the mean reduction in oocysts in the presence of the test IgG compared with the IgG (1 mg/ml) from pre-immune mouse sera (Pre) used as a negative control. % TBA is the percent reduction in the proportion of infected mosquitoes in the presence of the test IgG compared with pre-immune IgG negative control (Pre). The inset shows IgG1/IgG2a isotype ratios and antibody avidity index for sera evaluated by SMFA. Statistical analysis was performed using the Mann-Whitney U test (ns p>0.05; *p<0.05; **p<0.01; ***p<0.001; ****p<0.0001).

FIG. 6A-FIG. 6C shows that PfCSP mRNA-LNP induced responses in mice and protection against sporozoite challenge after four immunizations. Mice (n=5 per group) immunized with 3 μg, 10 μg, 30 μg PfCSP mRNA-LNPs, or co-immunized with 10 μg of both Pfs25- and PfCSP mRNA-LNPs (as per FIG. 1A) were challenged with PbPfCSP-GFPLuc sporozoites and progression of infection was monitored by microscope examination of giemsa-stained blood smears for 11 days following challenge. Pfs25 immunized mice were used as negative controls. FIG. 6A shows Kaplan-Meier survival curves represent the percent of mice without detectable parasitemia following sporozoite challenge. Mice were considered completely protected if no parasitemia was detected by day 11. Statistical analysis was performed using log rank (Mantel-Cox) test (*p<0.05; **p<0.01). Antibodies from sera, collected one-week prior to the second challenge, bleed B4 in FIG. 1A, were characterized for antibody isotype (FIG. 6B) by ELISA using IgG1, IgG2a, IgG2b, and IgG3 specific secondary antibodies. The optical densities (OD) of each isotype were normalized to the OD of total anti-mouse IgG. FIG. 6C shows that the avidity was characterized by antibody-antigen dissociation curves resulting from treatment with different concentrations of NaSCN from sera collected from the Bleeds, B3 and B4, in FIG. 1A. The avidity index was calculated as the concentration (M) of NaSCN that resulted in 50% dissociation of bound antibodies.

FIG. 7A-FIG. 7B shows transmission reducing activity of antibodies elicited by lower doses and varying immunization schedules of Pfs25 mRNA-LNP. FIG. 7A shows purified IgG from pooled sera (bleed B3) from Balb/c mice immunized with 0.1 μg, 1 μg, and 10 μg Pfs25 mRNA-LNPs (FIG. 2C) were evaluated at indicated final concentrations by SMFA. The inset shows the geometric mean antibody titers, avidity indices, and IgG1/IgG2a ratios for the serum evaluated by SMFA. FIG. 7B shows SMFA data for purified IgG in FB1, FB2, and B4 (FIG. 2A-FIG. 2B) from Balb/c mice immunized one, two and three doses of 10 μg of Pfs25 mRNA-LNP, respectively. The inset shows the antibody avidity indices and IgG1/IgG2a ratios for the serum evaluated by SMFA. Data points representing the oocyst counts from each mosquito, arithmetic means, and 95% confidence intervals are reported. N is the fraction of uninfected mosquitoes over the total number of mosquitoes dissected. % TRA is the mean reduction in oocysts in the presence of the test IgG compared with the IgG (1 mg/ml) from pre-immune mouse sera (Pre) used as a negative control. % TBA is the percent reduction in the proportion of infected mosquitoes in the presence of the test IgG compared with pre-immune mouse IgG negative control (Pre). Statistical analysis was performed using the Mann-Whitney U test (*p<0.05; **p<0.01; ***p<0.001; ****p<0.0001).

FIG. 8A-FIG. 8H show the evaluation of varying vaccine doses and immunization schedules of PfCSP mRNA-LNPs. Details of immunization and challenge are described schematically in FIG. 2A-FIG. 2C. FIG. 2A shows anti-PfCSP antibody responses from B3 and B5 (FIG. 2C) were analyzed by ELISA. FIG. 8B shows Kaplan-Meier survival curves representing outcome of sporozoite challenge after three immunizations with PfCSP mRNA-LNP (n=5 per groups). Mice after challenge were observed for 12 days by microscopic examination of giemsa-stained blood smears. Following the first challenge, mice were treated and immunized a fourth time and challenged as shown in FIG. 2C. FIG. 8C shows Kaplan-Meier survival curves after repeat challenge (N=5 per group). FIG. 8D shows antibody isotypes from the bleed (B5) and FIG. 8E shows antibody-antigen avidity index from the bleeds, B3 and B5 (FIG. 2C). FIG. 8F, FIG. 8G, and FIG. 8H show Kaplan-Meier survival curves for mice immunized with 10 μg PfCSP mRNA-LNP (N=5 per group except mice receiving 2 immunizations, N=4) and challenged after each immunization (FIG. 2A-FIG. 2C), anti-PfCSP antibody isotypes and anti-PfCSP avidity index, respectively. Mann-Whitney U test was used for statistical analysis of antibody titers, while log rank (Mantel Cox) test was used for the analysis of survival curves (*p<0.05; **p<0.01).

FIG. 9A-FIG. 9H shows evaluation of cellular responses in mice immunized with Pfs25 and PfCSP mRNA-LNPs. Groups of C57Bl/6 and Balb/c mice (N=5) were immunized with 1 μg and 3 μg of either Pfs25 or PfCSP mRNA-LNPs following one or two immunizations. Additional groups were immunized with two doses of 10 μg of rPfs25 and rPfCSP formulated with Alhydrogel, as positive controls. Anti-Pfs25 antibody responses (FIG. 9A) and anti-PfCSP (FIG. 9B) antibody responses evaluated by ELISA are reported. Germinal center B (GCB) cells and antigen specific B cells were quantified by flow cytometry. The reagents used are found in Table 1 and the gating strategy is reported in FIG. 10A-FIG. 10C. FIG. 9C-FIG. 9D show quantification of GCB cells and Pfs25-specific B cells respectively in Pfs25 mRNA-LNP immunized mice. FIG. 9E and FIG. 9F show the quantification of GCB cells and PfCSP-specific B cells, respectively in PfCSP immunized mice. Additionally, for the Pfs25 immunized mice, the harvested splenocytes were stimulated with a pool of overlapping peptides of Pfs25 for 6 h and assessed by flow cytometry for T cell responses. The frequency of stimulated cytokine-producing T cell subpopulations is reported. FIG. 9G shows CD4 T cell responses and table shows the CD8 T cell responses.

FIG. 10A-FIG. 10C shows the B cell gating strategy. FIG. 10A shows the gating scheme to identify isotype-switched (IgD-IgM-) B cells. FIG. 10B shows the identification of germinal center B cells (CD38-GL7+). FIG. 10C shows identification of Ag-binding B cells using fluorophore-conjugated protein probe.

FIG. 11 shows the T cell gating strategy to identify cytokine producing CD4 and CD8 T cells.

FIG. 12A-FIG. 12B show the cross-reactivity of antibody responses between Pfs25 and PfCSP antigens. Sera collected from the bleed, B3 (FIG. 1A), were analyzed by ELISA to determine PfCSP reactivity of Pfs25 mRNA-LNP immunized mice sera and vice-versa. Pre-immune sera were used as a negative control. Immune and pre-immune sera were tested at a fixed 1:500 dilution. Reactivity of sera to Pfs25 (FIG. 12A) and PfCSP (FIG. 12B) are shown.

FIG. 13A-FIG. 13D show the comparison of antibody responses in mice immunized with Pfs25 mRNA-LNPs, PfCSP mRNA-LNP and DNA plasmids encoding Pfs25 and PfCSP. Immunization and blood collection schedules are shown in FIG. 1A. Sera from bleeds at indicated time points were analyzed using ELISA to determine antigen-specific antibody titers reported as the geometric mean endpoint titers with 95% confidence intervals. FIG. 13A reports Pfs25-specific antibody titers of mice immunized with Pfs25 immunogens (n=5 for all groups except DNA group at time point B1, n=3 and at B4, n=4). FIG. 13B reports PfCSP-specific titers of mice immunized with PfCSP immunogens (n=5 for all groups). FIG. 13C and FIG. 13D report Pfs25-specific antibody titers (FIG. 13C) and PfCSP-specific antibody titers (FIG. 13D) of mice co-immunized with Pfs25 and PfCSP immunogens (n=5 for all groups except for DNA at time point B1, n=4, DNA at time points B3 and B4, n=3). ELISA data for mRNA immunization groups are the same as in FIG. 2 and FIG. 3; however, shown here for comparison with DNA immunization groups (shaded grey). Statistical analysis was performed using the Mann-Whitney U test (*p<0.05; **p<0.01).

FIG. 14A shows the plasmid map for VR1020 comprising PfCSP while FIG. 14B shows the plasmid map for VR1020 comprising Pfs25.

FIG. 15 provides the mapping of the 17 synthetic peptides (SEQ ID NO:14-SEQ ID NO:30) against the Pfs25 amino acid sequence (SEQ ID NO:01)

### BRIEF SUMMARY

Disclosed herein is a messenger RNA (mRNA) molecule, comprising one or more coding regions encoding at least one antigenic peptide or protein, wherein the at least one antigenic peptide or protein is present in one or more life cycle stages of a malarial parasite.

Disclosed herein is a messenger RNA (mRNA) molecule, comprising one or more coding regions encoding Plasmodium falciparum surface protein Pfs25.

Disclosed herein is a messenger RNA (mRNA) molecule, comprising one or more coding regions encoding at least Plasmodium falciparum surface protein circumsporozoite protein (PfCSP).

Disclosed herein is a messenger RNA (mRNA) molecule, comprising a first coding region encoding a first antigenic peptide or protein, and a second coding region encoding a second antigenic peptide or protein, wherein the first and second antigenic peptides or proteins are present in one or more life cycle stages of a malarial parasite.

Disclosed herein is a messenger RNA (mRNA) molecule, comprising a first coding region encoding comprises Plasmodium falciparum surface protein Pfs25, and a second coding region encoding Plasmodium falciparum surface protein circumsporozoite protein (PfCSP).

Disclosed herein is an RNA molecule comprising (i) a first coding region comprising a first open reading frame, and (ii) a second coding region comprising a second open reading frame.

Disclosed herein is an isolated DNA molecule comprising the sequence set forth in SEQ ID NO:09. Disclosed herein is an isolated DNA molecule comprising the sequence set forth in SEQ ID NO:10. Disclosed herein is an isolated DNA molecule comprising a sequence encoding Pfs25 and PfCSP. Disclosed herein is an isolated DNA molecule comprising the sequence set forth in SEQ ID NO:09 and the sequence set forth in SEQ ID NO:10.

Disclosed herein is an isolated DNA molecule comprising a first coding region encoding a first antigenic peptide or protein, and a second coding region encoding a second antigenic peptide or protein, wherein the first and second antigenic peptides or proteins are present in one or more life cycle stages of a malarial parasite.

Disclosed herein is an isolated DNA molecule comprising a first coding region encoding comprises Plasmodium falciparum surface protein Pfs25, and a second coding region encoding Plasmodium falciparum surface protein circumsporozoite protein (PfCSP).

Disclosed herein is pharmaceutical formulation comprising a disclosed mRNA molecule and a pharmaceutically acceptable carrier. Disclosed herein is pharmaceutical formulation comprising a disclosed mRNA vaccine and a pharmaceutically acceptable carrier,

Disclosed herein is pharmaceutical formulation comprising a mRNA molecule comprising one or more coding regions encoding at least one antigenic peptide or protein, wherein the at least one antigenic peptide or protein is present in one or more life cycle stages of a malarial parasite, and a pharmaceutically acceptable carrier.

Disclosed herein is an mRNA vaccine comprising any disclosed mRNA molecule.

Disclosed herein is an mRNA vaccine, comprising at least one messenger ribonucleic acid (mRNA) encoding at least one antigenic peptide or protein, wherein the at least one antigenic peptide or protein is present in one or more life cycle stages of a malarial parasite.

Disclosed herein is an mRNA vaccine, comprising at least one messenger ribonucleic acid (mRNA) encoding at least two antigenic peptides or proteins, wherein the at least two antigenic peptides or proteins are present in one or more life cycle stages of a malarial parasite.

Disclosed herein is an mRNA vaccine, comprising at least one messenger ribonucleic acid (mRNA) encoding at least one antigenic peptide or protein, wherein the at least one antigenic peptide or protein are present in one or more life cycle stages of a malarial parasite, wherein the disclosed mRNA molecule is encapsulated in a lipid nanoparticle.

Disclosed herein is an mRNA vaccine, comprising at least one messenger ribonucleic acid (mRNA) encoding at least two antigenic peptides or proteins, wherein the at least two antigenic peptide or proteins are present in one or more life cycle stages of a malarial parasite, wherein the disclosed mRNA molecule is encapsulated in a lipid nanoparticle.

Disclosed herein is an mRNA vaccine comprising a messenger RNA (mRNA) molecule encoding at least one antigenic peptide or protein, wherein the at least one antigenic peptide or protein is present in one or more life cycle stages of a malarial parasite, wherein the one or more life cycle stages of the malarial parasite comprises the sporozoite stage, the liver stage, the blood-stage, and the sexual-stage.

Disclosed herein is an mRNA vaccine comprising a messenger RNA (mRNA) molecule comprising one or more coding regions encoding Plasmodium falciparum surface protein Pfs25.

Disclosed herein is an mRNA vaccine comprising a messenger RNA (mRNA) molecule comprising one or more coding regions encoding at least Plasmodium falciparum surface protein circumsporozoite protein (PfCSP).

Disclosed herein is an mRNA vaccine comprising any disclosed mRNA molecule, wherein the mRNA vaccine comprises lipid nanoparticles (LNPs), and wherein the mRNA molecule is encapsulated in the LNPs.

Disclosed herein is an mRNA vaccine, comprising at least one messenger ribonucleic acid (mRNA) encoding at least two antigenic peptides or proteins, wherein the at least two antigenic peptides or proteins are present in one or more life cycle stages of a malarial parasite, wherein the mRNA vaccine comprises lipid nanoparticles (LNPs), and wherein the mRNA molecule is encapsulated in the LNPs.

Disclosed herein is an mRNA vaccine, comprising at least one messenger ribonucleic acid (mRNA) encoding at least one antigenic peptide or protein, wherein the at least one antigenic peptide or protein are present in one or more life cycle stages of a malarial parasite, wherein the disclosed mRNA molecule is encapsulated in a lipid nanoparticle, wherein the mRNA vaccine comprises lipid nanoparticles (LNPs), and wherein the mRNA molecule is encapsulated in the LNPs.

Disclosed herein is a method of inducing an antigen specific immune response, the method comprising administering to a subject in need thereof a disclosed mRNA vaccine in an amount effective to produce an antigen specific immune response in the subject.

Disclosed herein is a method of inducing an antigen specific immune response, the method comprising administering to a subject in need thereof an mRNA vaccine in an amount effective to produce an antigen specific immune response in the subject, wherein the immune response is specific to the peptide or protein encoded by the mRNA vaccine.

Disclosed herein is a method of inducing an antigen specific immune response, the method comprising administering to a subject a single disclosed mRNA vaccine or administering to a subject more than one disclosed mRNA vaccine in an amount effective to produce an antigen specific immune response in the subject, wherein the immune response is specific to the peptide or protein encoded by the mRNA vaccine.

Disclosed herein is a method of inducing an antigen specific immune response, the method comprising administering to a subject in need thereof a therapeutically effective an mRNA vaccine, wherein the mRNA vaccine comprises at least one messenger ribonucleic acid (mRNA) encoding at least two antigenic peptides or proteins, wherein the at least two antigenic peptides or proteins are present in one or more life cycle stages of a malarial parasite, wherein the immune response is specific to the at least two antigenic peptides or proteins.

Disclosed herein is a method of inducing an antigen specific immune response, the method comprising administering to a subject in need thereof a therapeutically effective an mRNA vaccine, wherein the mRNA vaccine comprises at least one messenger ribonucleic acid (mRNA) encoding at least two antigenic peptides or proteins, wherein the at least two antigenic peptides or proteins are present in one or more life cycle stages of a malarial parasite, wherein the mRNA vaccine comprises lipid nanoparticles (LNPs), and wherein the mRNA molecule is encapsulated in the LNPs, wherein the immune response is specific to the at least two antigenic peptides or proteins.

Disclosed herein is a method of inducing an antigen specific immune response, the method comprising administering to a subject in need thereof a therapeutically effective an mRNA vaccine, wherein the mRNA vaccine comprises at least one messenger ribonucleic acid (mRNA) encoding at least two antigenic peptides or proteins, wherein the at least two antigenic peptides or proteins are present in one or more life cycle stages of a malarial parasite, wherein the mRNA molecule is encapsulated in a lipid nanoparticle, wherein the mRNA vaccine comprises lipid nanoparticles (LNPs), and wherein the mRNA molecule is encapsulated in the LNPs, wherein the immune response is specific to the at least two antigenic peptides or proteins.

Disclosed herein is a method of interrupting malaria transmission, the method comprising administering to a subject in need thereof a disclosed mRNA vaccine in an amount effective to disrupt the sexual life cycle of the malarial parasite.

Disclosed herein is a method of interrupting malaria transmission, the method comprising administering to a subject in need thereof an mRNA vaccine in an amount effective to produce an antigen specific immune response in the subject, wherein the immune response is specific to the peptide or protein encoded by the mRNA vaccine, wherein the sexual life cycle of the malarial parasite is disrupted.

Disclosed herein is a method of interrupting malaria transmission, the method comprising administering to a subject a single disclosed mRNA vaccine or administering to a subject more than one disclosed mRNA vaccine in an amount effective to produce an antigen specific immune response in the subject, wherein the immune response is specific to the peptide or protein encoded by the mRNA vaccine, wherein the sexual life cycle of the malarial parasite is disrupted.

Disclosed herein is a method of interrupting malaria transmission, the method comprising administering to a subject in need thereof a therapeutically effective an mRNA vaccine, wherein the mRNA vaccine comprises at least one messenger ribonucleic acid (mRNA) encoding at least two antigenic peptides or proteins, wherein the at least two antigenic peptides or proteins are present in one or more life cycle stages of a malarial parasite, wherein the immune response is specific to the at least two antigenic peptides or proteins, wherein the sexual life cycle of the malarial parasite is disrupted.

Disclosed herein is a method of interrupting malaria transmission, the method comprising administering to a subject in need thereof a therapeutically effective an mRNA vaccine, wherein the mRNA vaccine comprises at least one messenger ribonucleic acid (mRNA) encoding at least two antigenic peptides or proteins, wherein the at least two antigenic peptides or proteins are present in one or more life cycle stages of a malarial parasite, wherein the disclosed mRNA molecule is encapsulated in a lipid nanoparticle, wherein the immune response is specific to the at least two antigenic peptides or proteins, wherein the sexual life cycle of the malarial parasite is disrupted.

Disclosed herein is a method of interrupting malaria transmission, the method comprising administering to a subject in need thereof a therapeutically effective an mRNA vaccine, wherein the mRNA vaccine comprises at least one messenger ribonucleic acid (mRNA) encoding at least two antigenic peptides or proteins, wherein the at least two antigenic peptides or proteins are present in one or more life cycle stages of a malarial parasite, wherein the mRNA vaccine comprises lipid nanoparticles (LNPs), and wherein the mRNA molecule is encapsulated in the LNPs, wherein the immune response is specific to the at least two antigenic peptides or proteins, wherein the sexual life cycle of the malarial parasite is disrupted.

Disclosed herein is a method of interrupting malaria transmission, the method comprising administering to a subject in need thereof a therapeutically effective an mRNA vaccine, wherein the mRNA vaccine comprises at least one messenger ribonucleic acid (mRNA) encoding at least two antigenic peptides or proteins, wherein the at least two antigenic peptides or proteins are present in one or more life cycle stages of a malarial parasite, wherein the mRNA molecule is encapsulated in a lipid nanoparticle, wherein the mRNA vaccine comprises lipid nanoparticles (LNPs), and wherein the mRNA molecule is encapsulated in the LNPs, wherein the immune response is specific to the at least two antigenic peptides or proteins, wherein the sexual life cycle of the malarial parasite is disrupted.

Disclosed herein is a method of treating and/or preventing malaria, the method comprising administering to a subject in need thereof a disclosed mRNA vaccine in an amount effective to treat and/or preventing malaria.

Disclosed herein is a method of treating and/or preventing malaria, the method comprising administering to a subject in need thereof an mRNA vaccine in an amount effective to produce an antigen specific immune response in the subject, wherein the immune response is specific to the peptide or protein encoded by the mRNA vaccine, wherein malaria is treated and/or prevented in the subject.

Disclosed herein is a method of treating and/or preventing malaria, the method comprising administering to a subject a single disclosed mRNA vaccine or administering to a subject more than one disclosed mRNA vaccine in an amount effective to produce an antigen specific immune response in the subject, wherein the immune response is specific to the peptide or protein encoded by the mRNA vaccine, wherein malaria is treated and/or prevented in the subject.

Disclosed herein is a method of treating and/or preventing malaria, the method comprising administering to a subject in need thereof a therapeutically effective an mRNA vaccine, wherein the mRNA vaccine comprises at least one messenger ribonucleic acid (mRNA) encoding at least two antigenic peptides or proteins, wherein the at least two antigenic peptides or proteins are present in one or more life cycle stages of a malarial parasite, wherein the immune response is specific to the at least two antigenic peptides or proteins, wherein malaria is treated and/or prevented in the subject.

Disclosed herein is a method of treating and/or preventing malaria, the method comprising administering to a subject in need thereof a therapeutically effective an mRNA vaccine, wherein the mRNA vaccine comprises at least one messenger ribonucleic acid (mRNA) encoding at least two antigenic peptides or proteins, wherein the at least two antigenic peptides or proteins are present in one or more life cycle stages of a malarial parasite, wherein the disclosed mRNA molecule is encapsulated in a lipid nanoparticle, wherein the immune response is specific to the at least two antigenic peptides or proteins, wherein malaria is treated and/or prevented in the subject.

Disclosed herein is a method of treating and/or preventing malaria, the method comprising administering to a subject in need thereof a therapeutically effective an mRNA vaccine, wherein the mRNA vaccine comprises at least one messenger ribonucleic acid (mRNA) encoding at least two antigenic peptides or proteins, wherein the at least two antigenic peptides or proteins are present in one or more life cycle stages of a malarial parasite, wherein the mRNA vaccine comprises lipid nanoparticles (LNPs), and wherein the mRNA molecule is encapsulated in the LNPs, wherein the immune response is specific to the at least two antigenic peptides or proteins, wherein malaria is treated and/or prevented in the subject.

Disclosed herein is a method of treating and/or preventing malaria, the method comprising administering to a subject in need thereof a therapeutically effective an mRNA vaccine, wherein the mRNA vaccine comprises at least one messenger ribonucleic acid (mRNA) encoding at least two antigenic peptides or proteins, wherein the at least two antigenic peptides or proteins are present in one or more life cycle stages of a malarial parasite, wherein the mRNA molecule is encapsulated in a lipid nanoparticle, wherein the mRNA vaccine comprises lipid nanoparticles (LNPs), and wherein the mRNA molecule is encapsulated in the LNPs, wherein the immune response is specific to the at least two antigenic peptides or proteins, wherein malaria is treated and/or prevented in the subject.

### DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Malaria

Malaria is an acute febrile illness caused by Plasmodium parasites, which are spread to people through the bites of infected female Anopheles mosquitoes. There are 5 parasite species that cause malaria in humans, and 2 of these species—P. falciparum and P. vivax—pose the greatest threat. P. falciparum is the deadliest malaria parasite and the most prevalent on the African continent. P. vivax is the dominant malaria parasite in most countries outside of sub-Saharan Africa.

The first symptoms—fever, headache, and chills—usually appear 10-15 days after the infective mosquito bite and may be mild and difficult to recognize as malaria. Left untreated, P. falciparum malaria can progress to severe illness and death within a period of 24 hours. In 2020, nearly half of the world's population was at risk of malaria. Some population groups are at considerably higher risk of contracting malaria and developing severe disease: infants, children under 5 years of age, pregnant women, and patients with HIV/AIDS, as well as people with low immunity moving to areas with intense malaria transmission such as migrant workers, mobile populations and travelers.

The malaria parasite develops both in humans and in the female Anopheles mosquitoes. The size and genetic complexity of the parasite mean that each infection presents thousands of antigens (proteins) to the human immune system. The parasite also changes through several life stages even while in the human host, presenting different antigens at different stages of its life cycle. Understanding which of these can be a useful target for vaccine development has been complicated. In addition, the parasite has developed a series of strategies that allow it to confuse, hide, and misdirect the human immune system.

Malaria infection begins when an infected female Anopheles mosquito bites a person, injecting Plasmodium parasites, in the form of sporozoites, into the bloodstream. The sporozoites pass quickly into the human liver. The sporozoites multiply asexually in the liver cells over the next 7 to 10 days, causing no symptoms. In an animal model, the parasites, in the form of merozoites, are released from the liver cells in vesicles, journey through the heart, and arrive in the lungs, where they settle within lung capillaries. The vesicles eventually disintegrate, freeing the merozoites to enter the blood phase of their development. In the bloodstream, the merozoites invade red blood cells (erythrocytes) and multiply again until the cells burst. Then they invade more erythrocytes. This cycle is repeated, causing fever each time parasites break free and invade blood cells. Some of the infected blood cells leave the cycle of asexual multiplication. Instead of replicating, the merozoites in these cells develop into sexual forms of the parasite, called gametocytes, that circulate in the blood stream. When a mosquito bites an infected human, it ingests the gametocytes, which develop further into mature sex cells called gametes. The fertilized female gametes develop into actively moving ookinetes that burrow through the mosquito's midgut wall and form oocysts on the exterior surface. Inside the oocyst, thousands of active sporozoites develop. The oocyst eventually bursts, releasing sporozoites into the body cavity that travel to the mosquito's salivary glands. The cycle of human infection begins again when the mosquito bites another person. (Baer K, et al. (2007) PLOS Pathogens. 11:e1).

### B. Pfs25

During sexual stages of the Plasmodium parasite, many surface proteins are synthesized de novo. P25 proteins are the major surface proteins of Plasmodium ookinetes, having molecular weight of 25 kDa. P25 proteins are present on ookinete surface of all known Plasmodium species. Many proteins have been identified as promising vaccine candidates against malaria including two proteins of P25 family. P25 proteins start expressing immediately after fertilization and continue to be expressed on zygote, ookinete, and young oocyst stages of Plasmodium. These proteins are present in abundance and are evenly distributed over the entire ookinete surface. Gene knockout experiments indicated that these proteins along with P28 proteins are necessary for the survival of parasite inside mosquito midgut. Structurally, all P25 proteins contain a signal sequence, four epidermal growth factor (EGF) domains, and a C-terminal glycosylphosphatidylinositol (GPI) anchor. EGF domains are known to be present especially in surface proteins where they participate in recognition and adhesion-like processes, which indicates that they play important roles in host parasite interactions. P25 proteins show very few sequence polymorphisms presumably because these proteins are never exposed to the vertebrate immune system.

Structure of these proteins can help in studying interaction of these proteins with respective transmission blocking antibodies and the receptors molecules present in mosquito midgut, which may help in understanding the biology of the parasite inside mosquito midgut. Homology modeling of eight P25 proteins indicated that all the mature P25 proteins are triangular flat molecules, each having four EGF domains arranged in the form of a triangle. A comparative analysis of the structures revealed that all P25 proteins have a structural scaffold of 22 conserved cysteines which form 11 disulphide bonds in all the members of the family. These disulphide bonds act as a skeleton for all the members of the P25 family leading to similarities in the overall structures of the family members. Dissimilarities in structures are primarily present in the loop regions of the P25 proteins. These loop regions are responsible for variable molecular recognition of P25 proteins among different species, whereas the conserved regions are responsible for similarity in functions of the P25 proteins among different Plasmodium species. A homologue of Pfs25 is Pvs25 (Plasmodium vivax), which is discussed infra.

### C. PfCSP

The P. falciparum circumsporozoite protein (PfCSP) covers the surface of the sporozoite and is critical to sporozoite development in the mosquito and cell invasion in the mammalian host. Proper folding of cysteine-containing proteins such as PfCSP, which includes two disulfide pairs and a fifth N-terminal cysteine, depends on the correct formation of disulfide bonds. PfCSP can be divided into three regions: the N-terminal region containing a highly conserved KLKQP motif (termed region I), which binds heparin sulfate proteoglycans, the central repeat region containing the NANP and NVDP protein motifs, and the C-terminal region containing the thrombospondin-like type I repeat (TSR). Whereas the central repeat region varies in length among P. falciparum isolates, the amino acid sequence of the repeat motif is conserved, indicating that they are structurally or functionally important, although not proven, and elicit strong immune responses. Irrespective of their biological roles, each of the individual regions provides an opportunity as a vaccine target, which supports the rationale to explore a recombinant PfCSP antigen as a vaccine candidate that encompasses as much of the native protein sequence as possible, including the full number of NANP and NVDP repeats. The N-terminal region contains an epitope that interacts with liver cells through heparin sulfate. Antibodies against this epitope are highly inhibitory in a sporozoite invasion assay. The junction between the N-terminal and central repeat regions contains an epitope targeted by potent neutralizing antibodies, one of which provides sterile protection in mice. The central repeat region is a major target for antibodies functional in in vitro and in vivo assays, and the C-terminal region contains B-cell epitopes and one or more CD8+ T-cell epitopes. A homologue of PfCSP is PvCSP (Plasmodium vivax), which is discussed infra.

### D. Relevant Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is +10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, the term “subject” refers to the target of administration, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have Malaria, be suspected of having Malaria, or be at risk of developing and/or acquiring Malaria. In an aspect, a subject can have Parkinson's disease, be suspected of having Parkinson's disease, or be at risk of developing and/or acquiring Parkinson's disease.

As used herein, the term “diagnosed” means having been subjected to an examination by a person of skill, for example, a physician, and found to have a condition that can be diagnosed or treated by one or more of the disclosed agents, disclosed therapeutic agents, disclosed pharmaceutical formulations, or a combination thereof, or by one or more of the disclosed methods. For example, “diagnosed with Malaria” means having been subjected to an examination by a person of skill, for example, a physician, and found to have a condition that can be treated by one or more of the disclosed mRNA molecules, disclosed mRNA vaccines, disclosed pharmaceutical formulations, or any combination thereof, or by one or more of the disclosed methods. For example, “suspected of having malaria” can mean having been subjected to an examination by a person of skill, for example, a physician, and found to have a condition that can likely be treated by one or more of the disclosed mRNA molecules, disclosed mRNA vaccines, disclosed pharmaceutical formulations, or any combination thereof, or by one or more of the disclosed methods. In an aspect, an examination can be physical, can involve various tests (e.g., blood tests, genotyping, biopsies, etc.) and assays (e.g., enzymatic assay), or a combination thereof.

A “patient” can refer to a subject that has been diagnosed with or is suspected of having malaria. In an aspect, a patient can refer to a subject that has been diagnosed with or is suspected of having malaria and is seeking treatment or receiving treatment for malaria.

As used herein, the phrase “identified to be in need of treatment for a disorder,” or the like, refers to selection of a subject based upon need for treatment of the disorder. For example, a subject can be identified as having a need for treatment of a disorder (e.g., such as malaria) based upon an earlier diagnosis by a person of skill and thereafter subjected to treatment for the disorder (e.g., malaria). In an aspect, the identification can be performed by a person different from the person making the diagnosis. In an aspect, the administration can be performed by one who performed the diagnosis.

As used herein, “antigen” can refer to a substance which may be recognized by the immune system, preferably by the adaptive immune system, and is capable of triggering an antigen-specific immune response, e.g., by formation of antibodies and/or antigen-specific T cells as part of an adaptive immune response. Typically, an antigen may be or may comprise a peptide or protein (e.g., Pfs25 and/or PfCSP) which may be presented by the MHC to T-cells. In the sense of the present invention an antigen may be the product of translation of a provided nucleic acid molecule, preferably an mRNA as defined herein. In this context, also fragments, variants and derivatives of peptides and proteins comprising at least one epitope are understood as antigen. In a preferred embodiment, an antigen may preferably be an antigen related to the COVID-19 coronavirus

As used herein, “epitope” (also called “antigen determinant”) can refer to T cell epitopes or parts of the proteins in the context of the present invention may comprise fragments preferably having a length of about 6 to about 20 or even more amino acids, e.g., fragments as processed and presented by MHC class I molecules, preferably having a length of about 8 to about 10 amino acids, or fragments as processed and presented by MHC class II molecules, preferably having a length of about 13 or more amino acids, wherein these fragments may be selected from any part of the amino acid sequence. These fragments are typically recognized by T cells in form of a complex consisting of the peptide fragment and an MHC molecule. B cell epitopes are typically fragments located on the outer surface of (native) protein or peptide antigens (e.g., Pfs25 and/or PfCSP), which can be recognized by antibodies.

As used herein, “vaccine” can be a prophylactic or therapeutic material providing at least one antigen or antigenic function. The antigen or antigenic function may stimulate the body's adaptive immune system to provide an adaptive immune response. A mRNA vaccine, as used herein, can be an mRNA having at least one open reading frame that can be translated by a cell or an organism provided with that mRNA. The product of this translation can be one or more peptides or proteins (or fragments thereof or variants thereof) that can act as an antigen, preferably as an immunogen.

As used herein, “inhibit,” “inhibiting”, and “inhibition” mean to diminish or decrease an activity, level, response, expression, condition, severity, disease, or other biological parameter (such as, for example, transmission). This can include, but is not limited to, the complete ablation of the activity, level, response, expression, condition, severity, disease, or other biological parameter (such as, for example, transmission). This can also include, for example, a 10% inhibition or reduction in the activity, level, response, condition, severity, disease, or other biological parameter (such as, for example, transmission) as compared to the native or control level (e.g., a subject not having malaria). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to native or control levels. In an aspect, the inhibition or reduction can be 0-25%, 25-50%, 50-75%, or 75-100% as compared to native or control levels. In an aspect, a native or control level can be a pre-disease or pre-disorder level.

The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder (such as malaria). In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating malaria can reduce the severity of an established disease in a subject by 1%-100% as compared to a control (such as, for example, an individual not having malaria). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of malaria. For example, treating malaria can reduce one or more symptoms of malaria in a subject by 1%-100% as compared to a control (such as, for example, an individual not having malaria). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established malaria. It is understood that treatment does not necessarily refer to a cure or complete ablation or eradication of malaria. However, in an aspect, treatment can refer to a cure or complete ablation or eradication of malaria.

The term “expression” or “expression of a coding sequence” (for example, a gene or a transgene) refer to the process by which the coded information of a nucleic acid transcriptional unit (such as, for example, mRNA)) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assays.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

As used herein, a “biomarker” refers to a defined characteristic that is measured as an indicator of normal biological processes, pathogenic processes, or response to an exposure of intervention. In an aspect, a biomarker can be diagnostic (i.e., detects or classifies a pathological condition), prognostic (i.e., predicts the probability of disease occurrence or progression), pharmacodynamic/responsive (i.e., identifies a change in response to a therapeutic intervention), predictive (i.e., predicts how an individual or subject might respond to a particular intervention or event). In an aspect, a biomarker can be diagnostic, prognostic, pharmacodynamic/responsive, and/or predictive at the same time. In an aspect, a biomarker can be diagnostic, prognostic, pharmacodynamic/responsive, and/or predictive at different times (e.g., first a biomarker can be diagnostic and then later, the same biomarker can be prognostic, pharmacodynamic/responsive, and/or predictive). A biomarker can be an objective measure that can be linked to a clinical outcome assessment. A biomarker can be used by the skilled person to make a clinical decision based on its context of use.

As used herein, “operably linked” means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

As used herein, a “regulatory element” can refer to promoters, enhancers, internal ribosomal entry sites (IRES), and other expression control elements (e.g., transcription termination signals, such as polyadenylation signals and poly-U sequences). Regulatory elements are discussed infra and can include those that direct constitutive expression of a nucleotide sequence in many types of host cells and those that direct expression of the nucleotide sequence only in certain host cells (e.g., tissue-specific regulatory sequences).

As known to the art, RNA binding proteins consist of multiple repetitive sequences that contain only a few specific basic domains. Structurally, common RNA-binding domains mainly include RNA-recognition motif (RRM), K homology (KH) domain, double-stranded RBD (dsRBD), cold-shock domain (CSD), arginine-glycine-glycine (RGG) motif, tyrosine-rich domain, and zinc fingers (ZnF) of the CCHC, CCCH, ZZ type etc. According to the different functions of RBPs in cells, RBPs can be divided into epithelial splicing regulatory proteins (ESRP1), cytoplasmic polyadenylation element binding protein family (CPEB1/2), Hu-antigen R (HuR), heterogeneous nuclear ribonucleoprotein family members (hnRNP A/D/H/K/M/E/L), insulin-like growth factor 2 mRNA family members (IMP1/2/3), zfh family of transcription factors (ZEB1/2), KH-type splicing regulatory protein (KHSRP), La ribonucleoprotein domain family members (LARP1/6/7), Lin-28 homolog proteins (Lin28), Musashi protein family (MSI1/2), pumilio protein family (PUM1/2), Quaking (QK), RNA-binding motif protein family (4/10/38/47), Src-associated substrate during mitosis of 68 kDa (SAM68), serine and arginine rich splicing factor (SRSF1/3), T cell intracellular antigens (TIA1/TIAR), and Upstream of N-Ras (UNR).

As used herein, “immune tolerance,” “immunological tolerance,” and “immunotolerance” refers to a state of unresponsiveness or blunted response of the immune system to substances (e.g., a disclosed mRNA molecule, a disclosed mRNA vaccine, a disclosed pharmaceutical formulation, or any combination thereof) that have the capacity to elicit an immune response in a subject. Immune tolerance is induced by prior exposure to a specific antigen. Immune tolerance can be determined in a subject by measuring antibodies against a particular antigenic peptide or protein (such as, for example, PfS25 or PfCSP). Low or absent antibody titers over time is an indicator of immune tolerance. For example, in an aspect, immune tolerance can be established by having IgG antibody titers of less than or equal to about 12,000, 11,500, 11,000, 10,500, 10,000, 9,500, 9,000, 8,500, 8,000, 7,500, 7,000, 6,500, or 6,000 within following gene therapy (such as the administration of the transgene encoding, for example, a missing, deficient, and/or mutant protein or enzyme).

As used herein, “peptide,” “polypeptide,” and “protein” are used interchangeably, and refer to a compound comprised of amino acid residues covalently linked by peptide bonds. A protein must contain at least two amino acids and there is no limitation on the maximum number of amino acids that can comprise a protein's sequence. The term “peptide” can refer to a short chain of amino acids including, for example, natural peptides, recombinant peptides, synthetic peptides, or any combination thereof. Proteins and peptides can include, for example, biologically active fragments, substantially homologous polypeptides, oligopeptides, homodimers, heterodimers, variants of polypeptides, modified polypeptides, derivatives, analogs, and fusion proteins, among others.

“Nucleic acid” or “oligonucleotide” or “polynucleotide” as used herein means at least two nucleotides covalently linked together. The depiction of a single strand can also define the sequence of the complementary strand. Thus, a nucleic acid can encompass the complementary strand of a depicted single strand. Many variants of a nucleic acid can be used for the same purpose as a given nucleic acid. Thus, a nucleic acid can encompass substantially identical nucleic acids and complements thereof. A single strand can provide a probe that can hybridize to a target sequence under stringent hybridization conditions. Thus, a nucleic acid can encompass a probe that hybridizes under stringent hybridization conditions. A nucleic acid can be single-stranded, or double-stranded, or can contain portions of both double-stranded and single-stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods. Also as used herein, the terms “nucleic acid,” “nucleic acid molecule,” “nucleic acid construct,” “nucleotide sequence”, and “polynucleotide” can refer to RNA or DNA that is linear or branched, single or double stranded, or a hybrid thereof. The term can encompass RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. A “synthetic” nucleic acid or polynucleotide, as used herein, refers to a nucleic acid or polynucleotide that is not found in nature but is constructed by the hand of man and therefore is not a product of nature.

A “polynucleotide” is a sequence of nucleotide bases, and may be RNA, DNA, or DNA-RNA hybrid sequences (including both naturally occurring and non-naturally occurring nucleotides).

A “fragment” or “portion” of a nucleotide sequence can be understood to mean a nucleotide sequence of reduced length relative (e.g., reduced by 1, 2, 3, 4, 5, 6, 7, 8, 9, 10, 11, 12, 13, 14, 15, 16, 17, 18, 19, 20 or more nucleotides) to a reference nucleic acid or nucleotide sequence and comprising, consisting essentially of, or consisting of a nucleotide sequence of contiguous nucleotides identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference nucleic acid or nucleotide sequence. Such a nucleic acid fragment or portion according to the disclosure can be, where appropriate, included in a larger polynucleotide of which it is a constituent.

A “fragment” or “portion” of an amino acid sequence can be understood to mean an amino acid sequence of reduced length relative (e.g., reduced by 1, 2, 3, 4, 5, 6, 7, 8, 9, 10, 11, 12, 13, 14, 15, 16, 17, 18, 19, 20, 30, 40, 50, 60, 70, 80, 90, 100, 150, 200, 250, or more amino acids) to a reference amino acid sequence and comprising, consisting essentially of, or consisting of an amino acid sequence of contiguous amino acids identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference amino acid sequence. Such an amino acid fragment or portion according to the disclosure can be, where appropriate, included in a larger amino acid sequence of which it is a constituent.

A “heterologous” or a “recombinant” nucleotide or amino acid sequence as used interchangeably herein can refer to a nucleotide or an amino acid sequence not naturally associated with a host cell into which it is introduced, including non-naturally occurring multiple copies of a naturally occurring nucleotide or amino acid sequence.

Different nucleic acids or proteins having homology can be referred to as “homologues”. The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the disclosed compositions and disclosed methods can comprise homologues to the disclosed nucleotide sequences and/or disclosed polypeptide sequences.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

As used herein, “promoter” or “promoters” are known to the art. Depending on the level and tissue-specific expression desired, a variety of promoter elements can be used. A promoter can be tissue-specific or ubiquitous and can be constitutive or inducible, depending on the pattern of the gene expression desired. A promoter can be native (endogenous) or foreign (exogenous) and can be a natural or a synthetic sequence. By foreign or exogenous, it is intended that the transcriptional initiation region is not found in the wild-type host into which the transcriptional initiation region is introduced.

As used herein, “codon optimization” can refer to a process of modifying a nucleic acid sequence for enhanced expression in the host cells of interest by replacing one or more codons or more of the native sequence with codons that are more frequently or most frequently used in the genes of that host cell while maintaining the native amino acid sequence. Various species exhibit particular bias for certain codons of a particular amino acid. As contemplated herein, genes can be tailored for optimal gene expression in a given organism based on codon optimization. Codon usage tables are readily available, for example, at the “Codon Usage Database.” Many methods and software tools for codon optimization have been reported previously. (See, for example, genomes.urv.es/OPTIMIZER/).

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing progression of malaria is intended. The words “prevent” and “preventing” and “prevention” also refer to prophylactic or preventative measures for protecting or precluding a subject (e.g., an individual) not having malaria or an malaria-related complication from progressing to that complication.

As used herein, the terms “administering” and “administration” refer to any method of providing one or more of the disclosed mRNA molecules, disclosed mRNA vaccines, disclosed pharmaceutical formulations, or any combination thereof to a subject. Such methods are well known to those skilled in the art and include, but are not limited to, the following routes: oral administration, transdermal administration, administration by inhalation, nasal administration, topical administration, in utero administration, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can be continuous or intermittent.

In an aspect, a “therapeutic agent” can be a “biologically active agent” or “biologic active agent” or “bioactive agent”, which refers to an agent that is capable of providing a local or systemic biological, physiological, or therapeutic effect in the biological system to which it is applied. For example, the bioactive agent can act to control infection or inflammation, enhance cell growth and tissue regeneration, control tumor growth, act as an analgesic, promote anti-cell attachment, and enhance bone growth, among other functions. Other suitable bioactive agents can include anti-viral agents, vaccines, hormones, antibodies (including active antibody fragments sFv, Fv, and Fab fragments), aptamers, peptide mimetics, functional nucleic acids, therapeutic proteins, peptides, or nucleic acids. Other bioactive agents include prodrugs, which are agents that are not biologically active when administered but, upon administration to a subject are converted to bioactive agents through metabolism or some other mechanism. Additionally, any of the compositions of the invention can contain combinations of two or more bioactive agents. It is understood that a biologically active agent can be used in connection with administration to various subjects, for example, to humans (i.e., medical administration) or to animals (i.e., veterinary administration). As used herein, the recitation of a biologically active agent inherently encompasses the pharmaceutically acceptable salts thereof.

In an aspect, a “therapeutic agent” can be any agent that effects a desired clinical outcome in a subject having malaria, suspected of having malaria, and/or likely to develop or acquire malaria. In an aspect, a disclosed therapeutic agent can be an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable.

By “determining the amount” is meant both an absolute quantification of a particular analyte (e.g., an mRNA sequence or an antibody specific for Pfs25 and/or PfCSP) or a determination of the relative abundance of a particular analyte (e.g., an amount as compared to a mRNA sequence or an antibody specific for Pfs25 and/or PfCSP). The phrase includes both direct or indirect measurements of abundance (e.g., individual mRNA transcripts may be quantified or the amount of amplification of an mRNA sequence under certain conditions for a certain period may be used a surrogate for individual transcript quantification) or both.

As used herein, “modifying the method” can comprise modifying or changing one or more features or aspects of one or more steps of a disclosed method. For example, in an aspect, a method can be altered by changing the amount of one or more of the disclosed mRNA molecules, disclosed mRNA vaccines, disclosed pharmaceutical formulations, or any combination thereof administered to a subject, or by changing the frequency of administration of one or more of the disclosed mRNA molecules, the disclosed mRNA vaccines, the disclosed pharmaceutical formulations, or any combination thereof to a subject, by changing the duration of time one or more of the disclosed mRNA molecules, disclosed mRNA vaccines, disclosed pharmaceutical formulations, or any combination thereof are administered to a subject, or by substituting for one or more of the disclosed components and/or reagents with a similar or equivalent component and/or reagent. The same applies to all disclosed therapeutic agents, immune modulators, immunosuppressive agents, proteosome inhibitors, etc.

In an aspect, a therapeutic agent can be a “drug” or a “vaccine” and means a molecule, group of molecules, complex or substance administered to an organism for diagnostic, therapeutic, preventative medical, or veterinary purposes. This term includes externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. Examples include but are not limited to a radiosensitizer, the combination of a radiosensitizer and a chemotherapeutic, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, carbonic anhydrase inhibitors, prostaglandin analogs, a combination of an alpha agonist and a beta blocker, a combination of a carbonic anhydrase inhibitor and a beta blocker, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, or a vaccine. In an aspect, the pharmaceutically active agent can be coumarin, albumin, bromolidine, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetominophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, timol hemihydrate, levobunolol hydrochloride, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists (i.e., alpha adrenergic receptor agonist) such as clonidine, brimonidine tartrate, and apraclonidine hydrochloride; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; prostaglandin analogs such as latanoprost, travoprost, and bimatoprost; cholinergics (i.e., acetylcholine receptor agonists) such as pilocarpine hydrochloride and carbachol; glutamate receptor agonists such as the N-methyl D-aspartate receptor agonist memantine; anti-Vascular endothelial growth factor (VEGF) aptamers such as pegaptanib; anti-VEGF antibodies (including but not limited to anti-VEGF-A antibodies) such as ranibizumab and bevacizumab; carbonic anhydrase inhibitors such as methazolamide, brinzolamide, dorzolamide hydrochloride, and acetazolamide; antiarrhythmic agents such as quinidine, lidocaine, tocainide hydrochloride, mexiletine hydrochloride, digoxin, verapamil hydrochloride, propafenone hydrochloride, flecaimide acetate, procainamide hydrochloride, moricizine hydrochloride, and diisopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hydrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides. It is understood that a pharmaceutically active agent can be used in connection with administration to various subjects, for example, to humans (i.e., medical administration) or to animals (i.e., veterinary administration). As used herein, the recitation of a pharmaceutically active agent inherently encompasses the pharmaceutically acceptable salts thereof.

“Sequence identity” and “sequence similarity” can be determined by alignment of two peptide or two nucleotide sequences using global or local alignment algorithms. Sequences may then be referred to as “substantially identical” or “essentially similar” when they are optimally aligned. For example, sequence similarity or identity can be determined by searching against databases such as FASTA, BLAST, etc., but hits should be retrieved and aligned pairwise to compare sequence identity. Two proteins or two protein domains, or two nucleic acid sequences can have “substantial sequence identity” if the percentage sequence identity is at least 70%, 75%, 80%, 85%, 90%, 95%, 98%, 99% or more, preferably 90%, 95%, 98%, 99% or more. Such sequences are also referred to as “variants” herein, e.g., other variants of glycogen branching enzymes and amylases. Sequences with substantial sequence identity do not necessarily have the same length and may differ in length. For example, sequences that have the same nucleotide sequence but of which one has additional nucleotides on the 3′- and/or 5′-side are 100% identical.

In an aspect, the skilled person can determine an efficacious dose, an efficacious schedule, and an efficacious route of administration for one or more of the disclosed mRNA molecules, disclosed mRNA vaccines, disclosed pharmaceutical formulations, or any combination thereof so as to treat or prevent malaria, malaria disease progression, and/or malaria transmission. In an aspect, the skilled person can also alter, change, or modify an aspect of an administering step to improve efficacy of one or more of the disclosed mRNA molecules, the disclosed mRNA vaccines, the disclosed pharmaceutical formulations, or any combination thereof. In an aspect, the skilled person can determine an efficacious dose, an efficacious schedule, and an efficacious route of administration for any disclosed mRNA molecule, disclosed mRNA vaccine, disclosed pharmaceutical formulation the disclosed mRNA molecules, the disclosed mRNA vaccines, the disclosed pharmaceutical formulations, or any combination thereof.

As used herein, “isolated” refers to a nucleic acid molecule or a nucleic acid sequence that has been substantially separated, produced apart from, or purified away from other biological components in the cell or tissue of an organism in which the component occurs, such as other cells, chromosomal and extrachromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins. Isolated proteins or nucleic acids, or cells containing such, in some examples are at least 50% pure, such as at least 75%, at least 80%, at least 90%, at least 95%, at least 98%, or at least 100% pure.

As used herein, “concurrently” means (1) simultaneously in time, or (2) at different times during the course of a common treatment schedule.

The term “contacting” as used herein refers to bringing one or more of the disclosed mRNA molecules, disclosed mRNA vaccines, disclosed pharmaceutical formulations, or any combination thereof together with a target area or intended target area in such a manner that the one or more of the disclosed mRNA molecules, disclosed mRNA vaccines, disclosed pharmaceutical formulations, or any combination thereof exert an effect on the intended target or targeted area either directly or indirectly. A target area or intended target area can be one or more of a subject's organs (e.g., lungs, heart, liver, kidney, brain, etc.). In an aspect, a target area or intended target area can be any cell or any organ infected by malaria (e.g., hepatocytes).

As used herein, “determining” can refer to measuring or ascertaining the presence and severity of malaria, or the development of malaria, or the transmission of malaria, or any combination thereof. Methods and techniques used to determine the presence and/or severity and/or transmission of malaria are typically known to the medical arts. For example, the art is familiar with the ways to identify and/or diagnose the presence, severity, or both of malaria. In an aspect, “determining” can also refer to measuring or ascertaining the level of one or more proteins or peptides in a biosample, or measuring or ascertaining the level or one or more RNAs or antibodies in a biosample. Methods and techniques for determining the expression and/or activity level of relevant proteins, peptides, mRNA, DNA, or any combination thereof known to the art and are disclosed herein.

As used herein, “effective amount” and “amount effective” can refer to an amount that is sufficient to achieve the desired result such as, for example, the treatment and/or prevention of malaria. As used herein, the terms “effective amount” and “amount effective” can refer to an amount that is sufficient to achieve the desired an effect on an undesired condition (malaria). For example, a “therapeutically effective amount” refers to an amount that is sufficient to achieve the desired therapeutic result or to have an effect on undesired symptoms, but is generally insufficient to cause adverse side effects. In an aspect, an effective amount can refer to the amount of a disclosed mRNA molecule or disclosed mRNA vaccine needed to induce an antigen specific immune response to an encoded peptide or protein (such as, for example, Pfs25 and/or PfCSP), or the amount needed to interrupt and/or disrupt the life cycle of the malarial parasite.

In an aspect, “therapeutically effective amount” means an amount of a disclosed mRNA molecule, a disclosed mRNA vaccine, a disclosed pharmaceutical formulation, or any combination thereof that (i) treats the particular disease, condition, or disorder (e.g., malaria), (ii) attenuates, ameliorates, or eliminates one or more symptoms of the particular disease, condition, or disorder (e.g., malaria), and/or (iii) delays the onset of one or more symptoms of the particular disease, condition, or disorder described herein (e.g., malaria). The specific therapeutically effective dose level for any particular patient will depend upon a variety of factors including the disorder being treated and the severity of the disorder; the disclosed mRNA molecules, disclosed mRNA vaccines, disclosed pharmaceutical formulations, or any combination thereof employed; the disclosed methods employed; the age, body weight, general health, sex and diet of the patient; the time of administration; the route of administration; the rate of excretion of the disclosed mRNA molecules, disclosed mRNA vaccines, disclosed pharmaceutical formulations, or any combination thereof employed; the duration of the treatment; drugs used in combination or coincidental with the disclosed mRNA molecules, disclosed mRNA vaccines, disclosed pharmaceutical formulations, or any combination thereof employed, and other like factors well known in the medical arts. For example, it is well within the skill of the art to start doses of the disclosed mRNA molecules, disclosed mRNA vaccines, disclosed pharmaceutical formulations, or any combination thereof at levels lower than those required to achieve the desired therapeutic effect and to gradually increase the dosage until the desired effect is achieved. If desired, then the effective daily dose can be divided into multiple doses for purposes of administration. Consequently, a single dose of the disclosed mRNA molecules, disclosed mRNA vaccines, disclosed pharmaceutical formulations, or any combination thereof can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition, such as, for example, malaria.

As used herein, the term “package insert” is used to refer to instructions customarily included in commercial packages of therapeutic products, that contain information about the indications, usage, dosage, administration, contraindications and/or warnings concerning the use of such therapeutic products.

As used herein, the term “in combination” in the context of the administration of one or more of the disclosed mRNA molecules, disclosed mRNA vaccines, disclosed pharmaceutical formulations, or any combination thereof includes the use of more than one therapy (e.g., additional therapeutic agents). Administration “in combination with” one or more additional therapeutic agents includes simultaneous (e.g., concurrent) and consecutive administration in any order. The use of the term “in combination” does not restrict the order in which therapies are administered to a subject. By way of non-limiting example, a first therapy (e.g., one or more of the disclosed mRNA molecules, disclosed mRNA vaccines, disclosed pharmaceutical formulations, or any combination thereof) may be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., one or more of the disclosed mRNA molecules, disclosed mRNA vaccines, disclosed pharmaceutical formulations, or any combination thereof or one or more additional therapeutic agents) to a subject having or diagnosed with malaria.

Disclosed are the components to be used to prepare the disclosed mRNA molecules, disclosed mRNA vaccines, disclosed pharmaceutical formulations, or any combination thereof as well the disclosed mRNA molecules, disclosed mRNA vaccines, disclosed pharmaceutical formulations, or any combination thereof used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific aspects or combination of aspects of the disclosed methods.

### Compositions

1. mRNA Molecules

Disclosed herein is a messenger RNA (mRNA) molecule, comprising one or more coding regions encoding at least one antigenic peptide or protein, wherein the at least one antigenic peptide or protein is present in one or more life cycle stages of a malarial parasite. In an aspect, the one or more life cycle stages of the malarial parasite can comprise the sporozoite stage, the liver stage, the blood-stage, and the sexual-stage.

Disclosed herein is a messenger RNA (mRNA) molecule, comprising one or more coding regions encoding Plasmodium falciparum surface protein Pfs25. Disclosed herein is a messenger RNA (mRNA) molecule, comprising a coding region encoding Plasmodium falciparum surface protein Pfs25, wherein the encoded Pfs25 comprises the sequence set forth in SEQ ID NO:01 or SEQ ID NO:07.

Disclosed herein is a messenger RNA (mRNA) molecule, comprising one or more coding regions encoding at least Plasmodium falciparum surface protein circumsporozoite protein (PfCSP). Disclosed herein is a messenger RNA (mRNA) molecule, comprising a coding region encoding Plasmodium falciparum surface protein circumsporozoite protein (PfCSP), wherein the encoded PfCSP comprises the sequence set forth in SEQ ID NO:02 or SEQ ID NO:08.

Disclosed herein is a messenger RNA (mRNA) molecule, comprising a first coding region encoding a first antigenic peptide or protein, and a second coding region encoding a second antigenic peptide or protein, wherein the first and second antigenic peptides or proteins are present in one or more life cycle stages of a malarial parasite.

In an aspect, the one or more life cycle stages of the malarial parasite can comprise the sporozoite stage, the liver stage, the blood-stage, and the sexual-stage.

In an aspect, a disclosed first antigenic peptide or protein can comprise Plasmodium falciparum surface protein Pfs25. In an aspect, a disclosed encoded Pfs25 can comprise the sequence set forth in SEQ ID NO:01 or SEQ ID NO:07. In an aspect, a disclosed second antigenic peptide or protein can comprise Plasmodium falciparum surface protein circumsporozoite protein (PfCSP). In an aspect, a disclosed encoded PfCSP can comprise the sequence set forth in SEQ ID NO:02 or SEQ ID NO:08.

Disclosed herein is a messenger RNA (mRNA) molecule, comprising a first coding region encoding comprises Plasmodium falciparum surface protein Pfs25, and a second coding region encoding Plasmodium falciparum surface protein circumsporozoite protein (PfCSP).

Disclosed herein is a messenger RNA (mRNA) molecule, comprising a first coding region encoding comprises Plasmodium falciparum surface protein Pfs25, and a second coding region encoding Plasmodium falciparum surface protein circumsporozoite protein (PfCSP), wherein the encoded Pfs25 comprises the sequence set forth in SEQ ID NO:01 or SEQ ID NO:07, and wherein the encoded PfCSP comprises the sequence set forth in SEQ ID NO:02 or SEQ ID NO:08.

In an aspect, the disclosed one or more coding regions can be codon-optimized for expression in a mammalian cell. In an aspect, a disclosed mammalian cell can be a human cell.

In an aspect, a disclosed malarial parasite can comprise Plasmodium falciparum (P. falciparum). In an aspect, a disclosed antigenic peptide or protein can comprise Pfs25, a fragment thereof, or a variant thereof. In an aspect, a disclosed antigenic peptide or protein can comprise circumsporozoite protein (PfCSP), a fragment thereof, or a variant thereof. In an aspect, a disclosed antigenic peptide or protein can comprise Pfs25, a fragment thereof, or a variant thereof, and PfCSP, a fragment thereof, a variant thereof, or any combination thereof. In an aspect, a disclosed antigenic peptide or protein can comprise Pfs and PfCSP.

In an aspect, a disclosed encoded Pfs25 can comprise the sequence set forth in SEQ ID NO:01. In an aspect, a disclosed encoded Pfs25 can comprise a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:01. In an aspect, a disclosed encoded Pfs25 can comprise a sequence having at least 40%-60%, at least 60%-80%, at least 80%-90%, or at least 90%-100% identity to the sequence set forth in SEQ ID NO:01.

In an aspect, a disclosed encoded Pfs25 can comprise the sequence set forth in any GenBank Accession No. XP_001347587.1, EUT85305.1, AAD39544.1, UFQ05488.1, UFQ05499.1, ETW42495.1, QOQ86355.1, QRV62119.1, QRV62061.1, QOQ86414.1, QOQ86416.1, QRV62092.1, 6PHB_E, 6AZZ_A, AIV43628.1, AIV43629.1, AIV43624.1, AIV43630.1, AIV43626.1, AIV43634.1, ASU08938.1, AKJ86874.1, AIV43627.1, ASU08940.1, ASU08939.1, ALF99893.1, ASU09129.1, ASU09128.1, ASU09130.1, UIT08865.1, UIT08866.1, ALF99891.1, ALF99892.1, AIV43625.1, ALF99890.1, or AKJ86873.1.

In an aspect, a disclosed encoded Pfs25 can comprise a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in GenBank Accession No. XP_001347587.1, EUT85305.1, AAD39544.1, UFQ05488.1, UFQ05499.1, ETW42495.1, QOQ86355.1, QRV62119.1, QRV62061.1, QOQ86414.1, QOQ86416.1, QRV62092.1, 6PHB_E, 6AZZ_A, AIV43628.1, AIV43629.1, AIV43624.1, AIV43630.1, AIV43626.1, AIV43634.1, ASU08938.1, AKJ86874.1, AIV43627.1, ASU08940.1, ASU08939.1, ALF99893.1, ASU09129.1, ASU09128.1, ASU09130.1, UIT08865.1, UIT08866.1, ALF99891.1, ALF99892.1, AIV43625.1, ALF99890.1, or AKJ86873.1.

In an aspect, a disclosed encoded PfCSP can comprise the sequence set forth in SEQ ID NO:02. In an aspect, a disclosed encoded PfCSP can comprise a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:02. In an aspect, a disclosed encoded PfCSP can comprise a sequence having at least 40%-60%, at least 60%-80%, at least 80%-90%, or at least 90%-100% identity to the sequence set forth in SEQ ID NO:02.

In an aspect, a disclosed encoded PfCSP can comprise the sequence set forth in GenBank Accession No. QOW08375.1, QOW08406.1, AAW78209.1, UFQ12080.1, QFZ93540.1, BAM84831.1, BAD73954.1, XP_001351122.1, BAM84814.1, AAW78190.1, AAW78206.1, UFQ12085.1, AAA29527.1, QFZ93502.1, UFQ12084.1, UFQ12082.1, QFZ93564.1, QFZ93554.1, AAW78200.1, AAF03135.1, AAW78193.1, ACO49498.1, AAN87576.1, ACO49324.1, BAM84859.1, AAN87615.1, ABF66070.1, BAM84771.1, ABF66086.1, QOW08441.1, QFZ93522.1, ABF66066.1, AAW78195.1, ABF66075.1, BAM84762.1, AAN87618.1, AAN87581.1, QOW08394.1, AAN87600.1, AAW78212.1, AW78211.1, AAN87622.1, ACO49332.1, ACO49330.1, KNG78490.1, AAN87620.1, BAD08405.1, QOW08419.1, ACO49375.1, AVC42000.1, P13814.1, ACO49327.1, QOW08472.1, QOW08469.1, AVC41974.1, AAA29547.1, BAM84971.1, ABF66089.1, ACO49331.1, AVC42006.1, ABF83989.1, BAM84962.1, ACO49325.1, ACO49339.1, AVC41971.1, AAA29543.1, AAA63422.1, ACO49409.1, BAM84856.1, ABF83997.1, AVC41992.1, ACO49410.1, or ABF83986.1.

In an aspect, a disclosed encoded PfCSP can comprise a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in GenBank Accession No. QOW08375.1, QOW08406.1, AAW78209.1, UFQ12080.1, QFZ93540.1, BAM84831.1, BAD73954.1, XP_001351122.1, BAM84814.1, AAW78190.1, AAW78206.1, UFQ12085.1, AAA29527.1, QFZ93502.1, UFQ12084.1, UFQ12082.1, QFZ93564.1, QFZ93554.1, AAW78200.1, AAF03135.1, AAW78193.1, ACO49498.1, AAN87576.1, ACO49324.1, BAM84859.1, AAN87615.1, ABF66070.1, BAM84771.1, ABF66086.1, QOW08441.1, QFZ93522.1, ABF66066.1, AAW78195.1, ABF66075.1, BAM84762.1, AAN87618.1, AAN87581.1, QOW08394.1, AAN87600.1, AAW78212.1, AW78211.1, AAN87622.1, ACO49330.1, KNG78490.1, AAN87620.1, BAD08405.1, QOW08419.1, ACO49375.1, AVC42000.1, P13814.1, ACO49327.1, QOW08472.1, QOW08469.1, AVC41974.1, AAA29547.1, BAM84971.1, ABF66089.1, ACO49331.1, AVC42006.1, ABF83989.1, BAM84962.1, ACO49325.1, ACO49339.1, AVC41971.1, AAA29543.1, AAA63422.1, ACO49409.1, BAM84856.1, ABF83997.1, AVC41992.1, ACO49410.1, or ABF83986.1.

In an aspect, a disclosed coding region can comprise an open reading frame. In an aspect, a disclosed open reading frame can encode the antigenic peptide or protein having the sequence set forth in SEQ ID NO:01 or SEQ ID NO:07. In an aspect, a disclosed open reading frame can encode the antigenic peptide or protein having the sequence set forth in SEQ ID NO:02 or SEQ ID NO:08.

In an aspect, a disclosed open reading frame can encode Pfs25 or PfCSP. In an aspect, a disclosed open reading frame can encode Pfs25 and PfCSP.

In an aspect, a disclosed open reading frame can comprise the sequence set forth in SEQ ID NO:09. In an aspect, a disclosed open reading frame can comprise a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:09. In an aspect, a disclosed open reading frame can comprise the sequence set forth in SEQ ID NO:10. In an aspect, a disclosed open reading frame can comprise a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:10.

Disclosed herein is a RNA molecule comprising (i) a first coding region comprising a first open reading frame, and (ii) a second coding region comprising a second open reading frame.

In an aspect, a disclosed first coding region can comprise the sequence set forth in SEQ ID NO:09, and wherein the second coding region comprises the sequence set forth in SEQ ID NO:10.

Disclosed herein is a mRNA molecule comprising a coding region, wherein the coding region encodes at least two, three, four, five, six, seven, eight, and more peptides or proteins (or fragment thereof, or derivative thereof), wherein the peptides or proteins are linked using an amino acid linker sequence. In an aspect, a disclosed amino acid linker can comprise a rigid linker, a flexible linker, a cleavable linker (e.g., self-cleaving peptides), or any combination thereof.

In an aspect, a disclosed mRNA molecule can be mono-, bi-, or multicistronic. The coding sequences in a bicistronic or multicistronic mRNA molecule can encode distinct peptides or proteins (such as, for example, Pfs25 and PfCSP).

Disclosed herein is a RNA molecule comprising (i) a first coding region encoding the antigenic peptide or protein having the sequence set forth in SEQ ID NO:01 or SEQ ID NO:07, and (ii) a second coding region encoding the antigenic peptide or protein having the sequence set forth in SEQ ID NO:02 or SEQ ID NO:08. In an aspect, a disclosed mRNA molecule can further comprise a N-terminal signal, a C-terminal GPI anchor, or the combination thereof. In an aspect, a disclosed N-terminal signal can comprise the sequence set forth in SEQ ID NO:03, SEQ ID NO:04, a fragment thereof, or a variant thereof. In an aspect, a disclosed C-terminal GPI anchor can comprise the sequence set forth in SEQ ID NO:05, SEQ ID NO:06, a fragment thereof, or a variant thereof. As known to the art, glycosylphosphatidylinositol (GPI) is a lipid anchor for many cell-surface proteins. In an aspect, a disclosed GPI anchor can represent a posttranslational modification of proteins with a glycolipid and is used ubiquitously in eukaryotes.

In an aspect, a disclosed RNA molecule can comprise a N-terminal signal having the sequence set forth in SEQ ID NO:03 and a C-terminal GPI anchor having the sequence set forth in SEQ ID NO:05. In an aspect, a disclosed RNA molecule can comprise a N-terminal signal having the sequence set forth in SEQ ID NO:04 and a C-terminal GPI anchor having the sequence set forth in SEQ ID NO:06. In an aspect, a disclosed RNA molecule can comprise an encoded Pfs25 having the sequence set forth in SEQ ID NO:01, a N-terminal signal having the sequence set forth in SEQ ID NO:03, and a C-terminal GPI anchor having the sequence set forth in SEQ ID NO:05. In an aspect, a disclosed RNA molecule can comprise an encoded PfCSP having the sequence set forth in SEQ ID NO:02, a N-terminal signal having the sequence set forth in SEQ ID NO:04, and a C-terminal GPI anchor having the sequence set forth in SEQ ID NO:06.

In an aspect, a disclosed RNA molecule can comprise one or more modified nucleosides. In an aspect, a modified nucleoside side can comprise a pseudouridine such as, for example, N1-methylpseudouridine-5′-triphosphate. Pseudouridines are known to the skilled person in the art. In an aspect, a disclosed RNA molecule can comprise a partial replacement or a complete replacement of native uridine with modified uridine.

In an aspect of a disclosed mRNA molecule can comprise one or more encoding regions comprising the sequence set forth in SEQ ID NO:07, a fragment thereof, or a variant thereof. In an aspect of a disclosed mRNA molecule can comprise one or more encoding regions comprising the sequence set forth in SEQ ID NO:08, a fragment thereof, or a variant thereof.

In an aspect of a disclosed mRNA molecule can comprise one or more mutations at one or more N-linked glycosylation site.

In an aspect of a disclosed mRNA molecule can further comprise at least one 5′-cap or 5′-cap structure, a 5′-UTR, a 3′-UTR, a poly-A region, or any combination thereof.

In an aspect, a disclosed 5′-cap or 5′-cap structure can comprise glyceryl, inverted deoxy abasic residue (moiety), 5 methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3 ‘-inverted nucleotide moiety, 3’-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′ phosphorothioate, phosphorodithioate, or bridging or non-bridging methylphosphonate moiety. In an aspect, a disclosed 5′-cap or 5′-cap structure can comprise CAP1 (additional methylation of the ribose of the adjacent nucleotide of m7GpppN), CAP2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse CAP analogue), modified ARCA (e.g. phosphothioate modified ARCA), inosine, Nl-methyl-guanosine, 2-fluoro-guanosine, 7-deaza-guanosine, 8-oxo-guanosine, 2-amino-guanosine, LNA-guanosine, or 2-azido-guanosine.

In an aspect, a disclosed polyA region can comprise at least 50 nucleotides, at least 60 nucleotides, at least 70 nucleotides, at least 80 nucleotides, at least 90 nucleotides, at least 100 nucleotides, at least 110 nucleotides, at least 120 nucleotides, or more than 120 nucleotides.

In an aspect, a disclosed 5′-UTR can comprise a Kozak sequence. In an aspect, a disclosed the 5′-UTR does not comprise a Kozak sequence. In an aspect, a disclosed 5′-UTR can comprise at least element that provides a translation regulatory activity. In an aspect, a disclosed translational regulatory activity can comprise enhancing and/or improving the translational fidelity of mRNA translation, increasing and/or improving the residence time of the 43 S pre-initiation complex (PIC) or ribosome at, or proximal to, the initiation codon, increasing and/or improving the initiation of polypeptide synthesis at or from the initiation codon, increasing and/or improving the amount of polypeptide translated from the full open reading frame, increasing and/or improving the fidelity of initiation codon decoding by the PIC or ribosome, inhibiting and/or reducing leaky scanning by the PIC or ribosome, inhibiting and/or reducing the rate of decoding the initiation codon by the PIC or ribosome, inhibiting and/or reducing the initiation of polypeptide synthesis at any codon within the mRNA other than the initiation codon, inhibiting and/or reducing the amount of polypeptide translated from any open reading frame within the mRNA other than the full open reading frame, inhibiting and/or reducing the production of aberrant translation products, or any combination thereof.

In an aspect, a disclosed mRNA molecule can further comprise one or more stabilizing elements. In an aspect, a disclosed stabilizing element can include, for example, a histone stem-loop. For example, a stem-loop binding protein (SLBP), a 32 kDa protein, is associated with the histone stem-loop at the 3′-end of the histone messages in both the nucleus and the cytoplasm. Its expression level is regulated by the cell cycle; it is peaks during the S-phase, when histone mRNA levels are also elevated. The protein appears to be necessary for efficient 3′-end processing of histone pre-mRNA by the U7 snRNP. SLBP continues to be associated with the stem-loop after processing, and then stimulates the translation of mature histone mRNAs into histone proteins in the cytoplasm.

In an aspect, a disclosed mRNA molecule can comprise an enhancer sequence and/or a promoter sequence, which can be modified or can be unmodified or which can be activated or can be inactivated.

In an aspect, a disclosed mRNA molecule can have one or more AU-rich sequences removed. In an aspect, these one or more AU-rich sequences (e.g., AURES) can be destabilizing sequences and/or can be found in the 3′-UTR. In an aspect, AURES can be removed from a disclosed mRNA molecule. In an aspect, AURES can remain in a disclosed mRNA molecule.

In an aspect, a disclosed mRNA molecule can be encapsulated in a lipid nanoparticle (LNP). In an aspect, a disclosed lipid nanoparticles can be prepared from materials comprising ionizable cationic lipids, phospholipid helper lipids, steroidal lipids, neutral phospholipids, phospholipid polyethylene glycol derivatives, or any combination thereof.

LNPs can include any cationic lipid suitable for forming a lipid nanoparticle. In an aspect, a disclosed cationic lipid can carry a net positive charge at about physiological pH. In an aspect, a disclosed cationic lipid can be an amino lipid. As used herein, the term “amino lipid” is meant to include those lipids having one or two fatty acid or fatty alkyl chains and an amino head group (including an alkylamino or dialkylamino group) that may be protonated to form a cationic lipid at physiological pH.

In an aspect, a disclosed cationic lipid can be, for example, N,N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy) propyl)-N,N,N-trimethylammonium chloride and 1,2-Dioleyloxy-3-trimethylaminopropane chloride salt), N-(1-(2,3-dioleyloxy) propyl)-N,N,N-trimethylammonium chloride (DOTMA), N,N-dimethyl-2,3-dioleyloxy) propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (γ-DLenDMA), 1,2-Dilinoleylcarbamoyloxy-3-dimethylaminopropane (DLin-C-DAP), 1,2-Dilinoleyoxy-3-(dimethylamino) acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Ci), 1,2-Dilinoleoyl-3-trimethylaminopropane chloride salt (DLin-TAP.CI), 1,2-Dilinoleyloxy-3-(N-methylpiperazino) propane (DLin-MPZ), or 3-(N,N-Dilinoleylamino)-1,2-propanediol (DLinAP), 3-(N,N-Dioleylamino)-1,2-propanediol (DOAP), 1,2-Dilinoleyloxo-3-(2-N,N-dimethylamino) ethoxypropane (DLin-EG-DM A), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl-4-(dimethylamino)butanoate (MC3), 1,1-(2-(4-(2-((2-(bis(2-hydroxydodecyl)amino)ethyl) (2-hydroxydodecyl)amino)ethyl) piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-DMA), (6Z,9Z,28Z,31Z)-heptatriaconta-6,9,28,31-tetraen-19-yl-4-(dimethylamino)butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N, N-dimethylbutan-1-amine (MC4 Ether), or any combination of any of the foregoing. Other cationic lipids include, but are not limited to, N,N-distearyl-N,N-dimethylammonium bromide (DDAB), 3P—(N—(N^{·},N^{·}-dimethylaminoethane)-carbamoyl) cholesterol (DC-Choi), N-(1-(2,3-dioleyloxy) propyl)-N-2-(sperminecarboxamido)ethyl)-N,N-dimethylammonium trifluoracetate (DOSPA), dioctadecylamidoglycyl carboxyspermine (DOGS), 1,2-dileoyl-sn-3-phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), and 2,2-Dilinoleyl-4-dimethylaminoethyl-[1,3]-dioxolane (XTC). Additionally, commercial preparations of cationic lipids can be used, such as LIPOFECTIN (including DOTMA and DOPE, available from GIBCO/BRL), and LIPOFECTAMINE (comprising DOSPA and DOPE, available from GIBCO/BRL).

In an aspect, a disclosed cationic lipid can be an amino lipid. Amino lipids can include those having alternative fatty acid groups and other dialkylamino groups, including those in which the alkyl substituents are different (e.g., N-ethyl-N-methylamino-, and N-propyl-N-ethylamino-). Generally, amino lipids having less saturated acyl chains are more easily sized, particularly when the complexes must be sized below about 0.3 microns, for purposes of filter sterilization. Amino lipids containing unsaturated fatty acids with carbon chain lengths in the range of C14 to C22 may be used. Other scaffolds can also be used to separate the amino group and the fatty acid or fatty alkyl portion of the amino lipid. Amino lipids include, but are not limited to, 1,2-dilinoleyoxy-3-(dimethylamino) acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-D-A), 1-linoleoyl-2-linoleyloxy-3-dimethylaminopropane (DLin-2-DMAP), 1,2-dilinoleyloxy-3-trimethylaminopropane chloride salt (DLin-TMA.CI), 1,2-dilinoleoyl-3-trimethylaminopropane chloride salt (DLin-TAP.CI), 1,2-dilinoleyloxy-3-(N-methylpiperazino) propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino) ethoxypropane (DLin-EG-DMA), and 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA); dilinoleyl-methyl-4-dimethylaminobutyrate (DLin-MC3-DMA); and C3 (US20100324120).

In an aspect, a disclosed phospholipid polyethylene glycol (PEG) derivatives can comprise 2-[(polyethylene glycol)-2000]-N, N-tetracosanyl acetamide (ALC-0159), 1,2-dimyristoyl-sn-glyceromethoxypolyethylene glycol (PEG-DMG), 1,2-distearoyl-sn-glycero-3-phosphoethanolamine-N-[amino (polyethylene glycol)] (PEG-DSPE), PEG-distearoyl glycerol (PEG-DSG), PEG-dipalmitoyl, one or more combinations of PEG-dioleyl, PEG-distearyl, PEG-diacylglycerol amide (PEG-DAG), PEG-dipalmitoyl phosphatidylethanolamine (PEG-DPPE), PEG-1, 2-dimyristoyloxypropyl-3-amine (PEG-c-DMA), DMG-PEG2000, or any combination thereof.

In an aspect, a disclosed neutral phospholipid can comprise 1,2-distearoyl-sn-glycerol-3-phosphocholine (DSPC), 1,2-dipalmitoyl-sn-glycerol-3-phosphocholine (DPPC), 1,2-dioleoyl-sn-glycerol-3-phosphoethanolamine (DOPE), 1,2-dipalmitoyl-sn-glycerol-3-phosphoethanolamine (DPPE), 1,2-dimyristoyl-sn-glycero-3-phosphoethanolamine (DMPE), 2-dioleoyl-sn-glycero-3-phospho-(1′-rac-glycerol) (DOPG), oleoyl phosphatidylcholine (POPC), 1-palmitoyl-2-oleoyl phosphatidylethanolamine (POPE), or any combination thereof.

In an aspect, a disclosed steroidal lipid can comprise avenasterol, β-sitosterol, brassicasterol, ergocalciferol, cholestanol, campesterol, cholesterol, coprosterol, dehydrocholesterol, desmosterol, dihydroergocalciferol, dihydrocholesterol, dihydroergosterol, echinosterol, epicholesterol, ergosterol, fucosterol, hexahydrosterol, hydroxycholesterol, and polypeptide-modified cholesterol; lanosterol, photosterol, fucosterol, sitostanol, sitosterol, stigmastanol, stigmasterol, cholic acid, glycocholic acid, taurocholic acid, deoxycholic acid, lithocholic acid, or any combination thereof.

In an aspect, a disclosed cationic lipid can be present in the lipid fraction in an amount of 20 to 60 mole percent. In an aspect, a disclosed neutral phospholipid can be present in the lipid fraction in an amount of 5 to 25 mole percent. In an aspect, a disclosed steroidal lipid can be present in the lipid fraction in an amount of 25 to 55 mole percent. In an aspect, a disclosed molar percentage of the polyethylene glycol (PEG)-lipid in the lipid component can be 0.1%-15%.

In an aspect, a disclosed lipid nanoparticle can be a commercially available lipid nanoparticle. In an aspect, for example, a disclosed nanoparticle can comprise a lipid nanoparticle disclosed in U.S. patent Ser. No. 10/221,127, which is incorporated by reference in its entirety for teachings of lipid nanoparticles or lipid nanoparticle formulations for delivery of a disclosed mRNA molecule.

In an aspect, a disclosed lipid nanoparticle can comprise an average particle size of about 10 nm to about 500 nm, about 10 nm to about 400 nm, about 10 nm to about 300 nm, about 10 to about 300 nm, about 10 to about 200 nm, about 10 nm to about 100 nm, about 10 nm to about 20 nm, about 20 nm to about 30 nm, about 30 nm to about 40 nm, about 40 nm to about 50 nm, about 50 nm to about 60 nm, about 60 nm to about 70 nm, about 70 nm to about 80 nm, about 80 nm to about 90 nm, or about 90 nm to about 100 nm. In an aspect, a disclosed lipid nanoparticle can comprise an average particle size of about 80 nm.

In an aspect, a disclosed lipid nanoparticle can comprise a polydispersity index of about 0.01 to about 0.10. In an aspect, a disclosed lipid nanoparticle can comprise a polydispersity index of about 0.01, about 0.02, about 0.03, about 0.04, about 0.05, about 0.06, about 0.07, about 0.08, about 0.09, or about 0.10. In an aspect, a disclose lipid nanoparticle can comprise a polydispersity index of about 0.02 to about 0.06.

In an aspect, a disclosed sequence can be CpG depleted and codon-optimized for expression in a human cell. In an aspect, “CpG-free” can mean completely free of CpGs or partially free of CpGs. In an aspect, “CpG-free” can mean “CpG-depleted”. In an aspect, “CpG-depleted” can mean “CpG-free”. In an aspect, “CpG-depleted” can mean completely depleted of CpGs or partially depleted of CpGs. In an aspect, “CpG-free” can mean “CpG-optimized” for a desired and/or ideal expression level. CpG depletion and/or optimization is known to the skilled person in the art.

In an aspect, a disclosed mRNA molecule encoding at least one antigenic peptide or protein can be codon-optimized. In an aspect, a disclosed sequence encoding Pfs25 and/or PfCSP can be codon-optimized.

In an aspect, a disclosed mRNA molecule can comprise a homologue of Pfs25 and/or PfCSP. In an aspect, for example, a homologue can be Pvs25 (Plasmodium vivax) or can be PvCSP (Plasmodium vivax). For example, disclosed herein is a messenger RNA (mRNA) molecule, comprising one or more coding regions encoding Plasmodium vivax ookinete surface protein (Pvs25). In an aspect, a disclosed encoded Pvs25 can comprise the sequence set forth in Accession No. ABS70936.1, AXG32346.1, XP_001608460.1, or a fragment thereof. In an aspect, a disclosed encoded Pvs25 can comprise any known sequence (e.g., a sequence identified using GenBank, for example). For example, disclosed herein is a messenger RNA (mRNA) molecule, comprising one or more coding regions encoding Plasmodium vivax circumsporozoite protein (PvCSP). In an aspect, a disclosed encoded PvCSP can comprise the sequence set forth in Accession No. BAO10695.1, BAO10696.1, or a fragment thereof. In an aspect, a disclosed encoded PvCSP can comprise any known sequence (e.g., a sequence identified using GenBank, for example).

### 2. Isolated DNA Molecules

Disclosed herein is an isolated DNA molecule comprising the sequence set forth in SEQ ID NO:09. Disclosed herein is an isolated DNA molecule comprising the sequence set forth in GenBank Accession No. AF193769.1.

Disclosed herein is an isolated DNA molecule comprising a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:09. Disclosed herein is an isolated DNA molecule comprising a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in GenBank Accession No. AF193769.1.

Disclosed herein is an isolated DNA molecule comprising a sequence encoding Pfs25, wherein the encoded Pfs25 comprises the sequence set forth in SEQ ID NO:01 or SEQ ID NO:07. Disclosed herein is an isolated DNA molecule comprising a sequence encoding Pfs25, wherein the encoded Pfs25 comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:01 or SEQ ID NO:07.

Disclosed herein is an isolated DNA molecule comprising the sequence set forth in SEQ ID NO:10. Disclosed herein is an isolated DNA molecule comprising the sequence set forth in GenBank Accession No. XM_001351086.1. Disclosed herein is an isolated DNA molecule comprising a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:10. Disclosed herein is an isolated DNA molecule comprising a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in GenBank Accession No. XM_001351086.1.

Disclosed herein is an isolated DNA molecule comprising a sequence encoding PfCSP, wherein the encoded PfCSP comprises the sequence set forth in SEQ ID NO:02 or SEQ ID NO:08. Disclosed herein is an isolated DNA molecule comprising a sequence encoding PfCSP, wherein the encoded PfCSP comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:02 or SEQ ID NO:08.

Disclosed herein is an isolated DNA molecule comprising a sequence encoding Pfs25 and PfCSP. Disclosed herein is an isolated DNA molecule comprising the sequence set forth in SEQ ID NO:09 and the sequence set forth in SEQ ID NO:10. Disclosed herein is an isolated DNA molecule comprising a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:09 and a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:10.

Disclosed herein is an isolated DNA molecule comprising the sequence set forth in GenBank Accession No. AF193769.1 and the sequence set forth in GenBank Accession No. XM_001351086.1. Disclosed herein is an isolated DNA molecule comprising a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in GenBank Accession No. AF193769.1 and a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in GenBank Accession No. XM_001351086.1.

Disclosed herein is an isolated DNA molecule comprising a sequence encoding Pfs25 and a sequence encoding PfCSP, wherein the encoded Pfs25 comprises the sequence set forth in SEQ ID NO:01 or SEQ ID NO:07, and wherein the encoded PfCSP comprises the sequence set forth in SEQ ID NO:02 or SEQ ID NO:08.

Disclosed herein is an isolated DNA molecule comprising a sequence encoding Pfs25 and a sequence encoding PfCSP, wherein the encoded Pfs25 comprises a sequence having at least 50%, 60%, 70%, 80%, 85%, 86%, 87%, 88%, 89%, 90%, 91%, 92%, 93%, 94%, 95%, 96%, 97%, 98%, or 99% identity to SEQ ID NO:01 or SEQ ID NO:07, and wherein the encoded PfCSP comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:02 or SEQ ID NO:08.

Disclosed herein is an isolated DNA molecule comprising a first coding region encoding a first antigenic peptide or protein, and a second coding region encoding a second antigenic peptide or protein, wherein the first and second antigenic peptides or proteins are present in one or more life cycle stages of a malarial parasite. In an aspect, life cycle stages of the malarial parasite can comprise the sporozoite stage, the liver stage, the blood-stage, and the sexual-stage.

In an aspect, a disclosed first antigenic peptide or protein can comprise Plasmodium falciparum surface protein Pfs25. In an aspect, a disclosed encoded Pfs25 can comprise the sequence set forth in SEQ ID NO:01 or SEQ ID NO:07. In an aspect, a disclosed second antigenic peptide or protein can comprise Plasmodium falciparum surface protein circumsporozoite protein (PfCSP). In an aspect, a disclosed encoded PfCSP can comprise the sequence set forth in SEQ ID NO:02 or SEQ ID NO:08.

Disclosed herein is an isolated DNA molecule comprising a first coding region encoding comprises Plasmodium falciparum surface protein Pfs25, and a second coding region encoding Plasmodium falciparum surface protein circumsporozoite protein (PfCSP). Disclosed herein is an isolated DNA molecule, comprising: a first coding region encoding comprises Plasmodium falciparum surface protein Pfs25, and a second coding region encoding Plasmodium falciparum surface protein circumsporozoite protein (PfCSP), wherein the encoded Pfs25 comprises the sequence set forth in SEQ ID NO:01 or SEQ ID NO:07, and wherein the encoded PfCSP comprises the sequence set forth in SEQ ID NO:02 or SEQ ID NO:08.

In an aspect, a disclosed isolated DNA molecule can comprise a coding region for a homologue of Pfs25 and/or PfCSP. In an aspect, for example, a homologue can be Pvs25 (Plasmodium vivax) or can be PvCSP (Plasmodium vivax). In an aspect, a disclosed isolated DNA molecule encoding Pvs25 can comprise the sequence set forth in Accession No. MG496335.1, MG496322.1, or a fragment thereof. In an aspect, a disclosed isolated DNA molecule encoding Pvs25 can comprise any known sequence (e.g., a sequence identified using GenBank, for example). In an aspect, a disclosed isolated DNA molecule encoding PvCSP can comprise the sequence set forth in Accession No. EU031824.1, AB539044, or a fragment thereof. In an aspect, a disclosed isolated DNA molecule encoding PvCSP can comprise any known sequence (e.g., a sequence identified using GenBank, for example).

### 3. Pharmaceutical Formulations

Disclosed herein is pharmaceutical formulation comprising a disclosed mRNA molecule and a pharmaceutically acceptable carrier. Disclosed herein is pharmaceutical formulation comprising a disclosed mRNA vaccine and a pharmaceutically acceptable carrier,

Disclosed herein is pharmaceutical formulation comprising a mRNA molecule comprising one or more coding regions encoding at least one antigenic peptide or protein, wherein the at least one antigenic peptide or protein is present in one or more life cycle stages of a malarial parasite, and a pharmaceutically acceptable carrier. In an aspect, the disclosed mRNA can be encapsulated in LNPs.

Disclosed herein is pharmaceutical formulation comprising a messenger RNA (mRNA) molecule comprising one or more coding regions encoding Plasmodium falciparum surface protein Pfs25, and a pharmaceutically acceptable carrier. In an aspect, the disclosed mRNA can be encapsulated in LNPs.

Disclosed herein is pharmaceutical formulation comprising a messenger RNA (mRNA) molecule comprising a coding region encoding Plasmodium falciparum surface protein Pfs25, wherein the encoded Pfs25 comprises the sequence set forth in SEQ ID NO:01 or SEQ ID NO:07, and a pharmaceutically acceptable carrier. In an aspect, the disclosed mRNA can be encapsulated in LNPs.

Disclosed herein is pharmaceutical formulation comprising a messenger RNA (mRNA) molecule comprising one or more coding regions encoding at least Plasmodium falciparum surface protein circumsporozoite protein (PfCSP), and a pharmaceutically acceptable carrier. In an aspect, the disclosed mRNA can be encapsulated in LNPs.

Disclosed herein is pharmaceutical formulation comprising a messenger RNA (mRNA) molecule comprising a coding region encoding Plasmodium falciparum surface protein circumsporozoite protein (PfCSP), wherein the encoded PfCSP comprises the sequence set forth in SEQ ID NO:02 or SEQ ID NO:08, and a pharmaceutically acceptable carrier. In an aspect, the disclosed mRNA can be encapsulated in LNPs.

Disclosed herein is pharmaceutical formulation comprising is a messenger RNA (mRNA) molecule comprising a first coding region encoding a first antigenic peptide or protein, and a second coding region encoding a second antigenic peptide or protein, wherein the first and second antigenic peptides or proteins are present in one or more life cycle stages of a malarial parasite, and a pharmaceutically acceptable carrier. In an aspect, the disclosed mRNA can be encapsulated in LNPs.

Disclosed herein is pharmaceutical formulation comprising a messenger RNA (mRNA) molecule comprising a first coding region encoding comprises Plasmodium falciparum surface protein Pfs25, and a second coding region encoding Plasmodium falciparum surface protein circumsporozoite protein (PfCSP), and a pharmaceutically acceptable carrier. In an aspect, the disclosed mRNA can be encapsulated in LNPs.

Disclosed herein is pharmaceutical formulation comprising a messenger RNA (mRNA-) molecule comprising a first coding region encoding comprises Plasmodium falciparum surface protein Pfs25, and a second coding region encoding Plasmodium falciparum surface protein circumsporozoite protein (PfCSP), wherein the encoded Pfs25 comprises the sequence set forth in SEQ ID NO:01 or SEQ ID NO:07, and wherein the encoded PfCSP comprises the sequence set forth in SEQ ID NO:02 or SEQ ID NO:08, and a pharmaceutically acceptable carrier. In an aspect, the disclosed mRNA can be encapsulated in LNPs.

Disclosed herein is pharmaceutical formulation comprising a mRNA molecule comprising a coding region, wherein the coding region encodes at least two, three, four, five, six, seven, eight, and more peptides or proteins (or fragment thereof, or derivative thereof), wherein the peptides or proteins are linked using an amino acid linker sequence, and a pharmaceutically acceptable carrier. In an aspect, the disclosed mRNA can be encapsulated in LNPs.

Disclosed herein is pharmaceutical formulation comprising a mRNA molecule comprising (i) a first coding region encoding the antigenic peptide or protein having the sequence set forth in SEQ ID NO:01 or SEQ ID NO:07, and (ii) a second coding region encoding the antigenic peptide or protein having the sequence set forth in SEQ ID NO:02 or SEQ ID NO:08, and a pharmaceutically acceptable carrier. In an aspect, the disclosed mRNA can be encapsulated in LNPs.

In an aspect, a disclosed pharmaceutical formulation can comprise a mRNA molecule encoding a homologue of Pfs25 and/or PfCSP. In an aspect, for example, a homologue can be Pvs25 (Plasmodium vivax) or can be PvCSP (Plasmodium vivax), both of which are discussed supra.

In an aspect, a disclosed formulation can comprise (i) one or more active agents, (ii) biologically active agents, (iii) one or more pharmaceutically active agents, (iv) one or more immune-based therapeutic agents, (v) one or more clinically approved agents, or (vi) any combination thereof.

In an aspect, a disclosed pharmaceutical formulation can treat and/or prevent malaria disease progression, malaria infection, malaria transmission, or any combination thereof.

In an aspect, a disclosed pharmaceutically acceptable carrier can refer to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a disclosed pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen.

In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

In an aspect, a disclosed excipient can refer to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

4. mRNA Vaccines

Disclosed herein is an mRNA vaccine comprising any disclosed mRNA molecule.

Disclosed herein is an mRNA vaccine comprising any disclosed mRNA molecule, wherein the disclosed mRNA molecule is encapsulated in a disclosed lipid nanoparticle.

Disclosed herein is an mRNA vaccine comprising any disclosed mRNA molecule, wherein the disclosed mRNA molecule is encapsulated in a disclosed lipid nanoparticle.

Disclosed herein is an mRNA vaccine, comprising at least one messenger ribonucleic acid (mRNA) encoding at least one antigenic peptide or protein, wherein the at least one antigenic peptide or protein is present in one or more life cycle stages of a malarial parasite.

Disclosed herein is an mRNA vaccine, comprising at least one messenger ribonucleic acid (mRNA) encoding at least two antigenic peptides or proteins, wherein the at least two antigenic peptides or proteins are present in one or more life cycle stages of a malarial parasite.

Disclosed herein is an mRNA vaccine, comprising at least one messenger ribonucleic acid (mRNA) encoding at least one antigenic peptide or protein, wherein the at least one antigenic peptide or protein are present in one or more life cycle stages of a malarial parasite, wherein the disclosed mRNA molecule is encapsulated in a lipid nanoparticle.

Disclosed herein is an mRNA vaccine, comprising at least one messenger ribonucleic acid (mRNA) encoding at least two antigenic peptides or proteins, wherein the at least two antigenic peptide or proteins are present in one or more life cycle stages of a malarial parasite, wherein the disclosed mRNA molecule is encapsulated in a lipid nanoparticle.

Disclosed herein is an mRNA vaccine comprising a messenger RNA (mRNA) molecule encoding at least one antigenic peptide or protein, wherein the at least one antigenic peptide or protein is present in one or more life cycle stages of a malarial parasite, wherein the one or more life cycle stages of the malarial parasite comprises the sporozoite stage, the liver stage, the blood-stage, and the sexual-stage.

Disclosed herein is an mRNA vaccine comprising a messenger RNA (mRNA) molecule comprising one or more coding regions encoding Plasmodium falciparum surface protein Pfs25.

Disclosed herein is an mRNA vaccine comprising a messenger RNA (mRNA) molecule comprising a coding region encoding Plasmodium falciparum surface protein Pfs25, wherein the encoded Pfs25 comprises the sequence set forth in SEQ ID NO:01 or SEQ ID NO:07.

Disclosed herein is an mRNA vaccine comprising a messenger RNA (mRNA) molecule comprising one or more coding regions encoding at least Plasmodium falciparum surface protein circumsporozoite protein (PfCSP).

Disclosed herein is an mRNA vaccine comprising a messenger RNA (mRNA) molecule comprising a coding region encoding Plasmodium falciparum surface protein circumsporozoite protein (PfCSP), wherein the encoded PfCSP comprises the sequence set forth in SEQ ID NO:02 or SEQ ID NO:08.

Disclosed herein is an mRNA vaccine comprising any disclosed mRNA molecule, wherein the mRNA vaccine comprises lipid nanoparticles (LNPs), and wherein the mRNA molecule is encapsulated in the LNPs.

Disclosed herein is an mRNA vaccine, comprising at least one messenger ribonucleic acid (mRNA) encoding at least one antigenic peptide or protein, wherein the at least one antigenic peptide or protein is present in one or more life cycle stages of a malarial parasite, wherein the mRNA vaccine comprises lipid nanoparticles (LNPs), and wherein the mRNA molecule is encapsulated in the LNPs.

Disclosed herein is an mRNA vaccine, comprising at least one messenger ribonucleic acid (mRNA) encoding at least two antigenic peptides or proteins, wherein the at least two antigenic peptides or proteins are present in one or more life cycle stages of a malarial parasite, wherein the mRNA vaccine comprises lipid nanoparticles (LNPs), and wherein the mRNA molecule is encapsulated in the LNPs.

Disclosed herein is an mRNA vaccine, comprising at least one messenger ribonucleic acid (mRNA) encoding at least one antigenic peptide or protein, wherein the at least one antigenic peptide or protein are present in one or more life cycle stages of a malarial parasite, wherein the disclosed mRNA molecule is encapsulated in a lipid nanoparticle, wherein the mRNA vaccine comprises lipid nanoparticles (LNPs), and wherein the mRNA molecule is encapsulated in the LNPs.

Disclosed herein is an mRNA vaccine, comprising at least one messenger ribonucleic acid (mRNA) encoding at least two antigenic peptides or proteins, wherein the at least two antigenic peptide or proteins are present in one or more life cycle stages of a malarial parasite, wherein the disclosed mRNA molecule is encapsulated in a lipid nanoparticle, wherein the mRNA vaccine comprises lipid nanoparticles (LNPs), and wherein the mRNA molecule is encapsulated in the LNPs.

Disclosed herein is an mRNA vaccine comprising a messenger RNA (mRNA) molecule encoding at least one antigenic peptide or protein, wherein the at least one antigenic peptide or protein is present in one or more life cycle stages of a malarial parasite, wherein the one or more life cycle stages of the malarial parasite comprises the sporozoite stage, the liver stage, the blood-stage, and the sexual-stage, wherein the mRNA vaccine comprises lipid nanoparticles (LNPs), and wherein the mRNA molecule is encapsulated in the LNPs.

Disclosed herein is an mRNA vaccine comprising a messenger RNA (mRNA) molecule comprising one or more coding regions encoding Plasmodium falciparum surface protein Pfs25, wherein the mRNA vaccine comprises lipid nanoparticles (LNPs), and wherein the mRNA molecule is encapsulated in the LNPs.

Disclosed herein is an mRNA vaccine comprising a messenger RNA (mRNA) molecule comprising a coding region encoding Plasmodium falciparum surface protein Pfs25, wherein the encoded Pfs25 comprises the sequence set forth in SEQ ID NO:01 or SEQ ID NO:07, wherein the mRNA vaccine comprises lipid nanoparticles (LNPs), and wherein the mRNA molecule is encapsulated in the LNPs.

Disclosed herein is an mRNA vaccine comprising a messenger RNA (mRNA) molecule comprising one or more coding regions encoding at least Plasmodium falciparum surface protein circumsporozoite protein (PfCSP), wherein the mRNA vaccine comprises lipid nanoparticles (LNPs), and wherein the mRNA molecule is encapsulated in the LNPs.

Disclosed herein is an mRNA vaccine comprising a messenger RNA (mRNA) molecule comprising a coding region encoding Plasmodium falciparum surface protein circumsporozoite protein (PfCSP), wherein the encoded PfCSP comprises the sequence set forth in SEQ ID NO:02 or SEQ ID NO:08, wherein the mRNA vaccine comprises lipid nanoparticles (LNPs), and wherein the mRNA molecule is encapsulated in the LNPs.

Disclosed herein is an mRNA vaccine comprising a messenger RNA (mRNA) molecule, comprising: a first coding region encoding a first antigenic peptide or protein, and a second coding region encoding a second antigenic peptide or protein, wherein the first and second antigenic peptides or proteins are present in one or more life cycle stages of a malarial parasite.

In an aspect, a disclosed one or more life cycle stages of the malarial parasite can comprise the sporozoite stage, the liver stage, the blood-stage, and the sexual-stage.

In an aspect, a disclosed first antigenic peptide or protein can comprise Plasmodium falciparum surface protein Pfs25. In an aspect, a disclosed encoded Pfs25 can comprise the sequence set forth in SEQ ID NO:01 or SEQ ID NO:07. In an aspect, a disclosed second antigenic peptide or protein can comprise Plasmodium falciparum surface protein circumsporozoite protein (PfCSP). In an aspect, a disclosed encoded PfCSP can comprises the sequence set forth in SEQ ID NO:02 or SEQ ID NO:08.

Disclosed herein is an mRNA vaccine comprising a messenger RNA (mRNA) molecule, comprising: a first coding region encoding comprises Plasmodium falciparum surface protein Pfs25, and a second coding region encoding Plasmodium falciparum surface protein circumsporozoite protein (PfCSP).

Disclosed herein is an mRNA vaccine comprising mRNA vaccine comprising a messenger RNA (mRNA) molecule, comprising: a first coding region encoding comprises Plasmodium falciparum surface protein Pfs25, and a second coding region encoding Plasmodium falciparum surface protein circumsporozoite protein (PfCSP), wherein the encoded Pfs25 comprises the sequence set forth in SEQ ID NO:01 or SEQ ID NO:07, and wherein the encoded PfCSP comprises the sequence set forth in SEQ ID NO:02 or SEQ ID NO:08.

In an aspect of a disclosed mRNA vaccine, the disclosed one or more coding regions can be codon-optimized for expression in a mammalian cell. In an aspect, a disclosed mammalian cell can be a human cell.

In an aspect of a disclosed mRNA vaccine, a disclosed malarial parasite can comprise Plasmodium falciparum (P. falciparum). In an aspect, a disclosed antigenic peptide or protein can comprise Pfs25, a fragment thereof, or a variant thereof. In an aspect, a disclosed antigenic peptide or protein can comprise circumsporozoite protein (PfCSP), a fragment thereof, or a variant thereof. In an aspect, a disclosed antigenic peptide or protein can comprise Pfs25, a fragment thereof, or a variant thereof, and PfCSP, a fragment thereof, a variant thereof, or any combination thereof. In an aspect, a disclosed antigenic peptide or protein can comprise Pfs and PfCSP.

In an aspect of a disclosed mRNA vaccine, a disclosed encoded Pfs25 can comprise the sequence set forth in SEQ ID NO:01. In an aspect, a disclosed encoded Pfs25 can comprise a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:01. In an aspect, a disclosed encoded Pfs25 can comprise a sequence having at least 40%-60%, at least 60%-80%, at least 80%-90%, or at least 90%-100% identity to the sequence set forth in SEQ ID NO:01.

In an aspect of a disclosed mRNA vaccine, a disclosed encoded Pfs25 can comprise the sequence set forth in any GenBank Accession No. XP_001347587.1, EUT85305.1, AAD39544.1, UFQ05488.1, UFQ05499.1, ETW42495.1, QOQ86355.1, QRV62119.1, QRV62061.1, QOQ86414.1, QOQ86416.1, QRV62092.1, 6PHB_E, 6AZZ_A, AIV43628.1, AIV43629.1, AIV43624.1, AIV43630.1, AIV43626.1, AIV43634.1, ASU08938.1, AKJ86874.1, AIV43627.1, ASU08940.1, ASU08939.1, ALF99893.1, ASU09129.1, ASU09128.1, ASU09130.1, UIT08865.1, UIT08866.1, ALF99891.1, ALF99892.1, AIV43625.1, ALF99890.1, or AKJ86873.1.

In an aspect of a disclosed mRNA vaccine, a disclosed encoded Pfs25 can comprise a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in GenBank Accession No. XP 001347587.1, EUT85305.1, AAD39544.1, UFQ05488.1, UFQ05499.1, ETW42495.1, QOQ86355.1, QRV62119.1, QRV62061.1, QOQ86414.1, QOQ86416.1, QRV62092.1, 6PHB_E, 6AZZ_A, AIV43628.1, AIV43629.1, AIV43624.1, AIV43630.1, AIV43626.1, AIV43634.1, ASU08938.1, AKJ86874.1, AIV43627.1, ASU08940.1, ASU08939.1, ALF99893.1, ASU09129.1, ASU09128.1, ASU09130.1, UIT08865.1, UIT08866.1, ALF99891.1, ALF99892.1, AIV43625.1, ALF99890.1, or AKJ86873.1.

In an aspect of a disclosed mRNA vaccine, a disclosed encoded PfCSP can comprise the sequence set forth in SEQ ID NO:02. In an aspect, a disclosed encoded PfCSP can comprise a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:02. In an aspect, a disclosed encoded PfCSP can comprise a sequence having at least 40%-60%, at least 60%-80%, at least 80%-90%, or at least 90%-100% identity to the sequence set forth in SEQ ID NO:02.

In an aspect of a disclosed mRNA vaccine, a disclosed encoded PfCSP can comprise the sequence set forth in GenBank Accession No. QOW08375.1, QOW08406.1, AAW78209.1, UFQ12080.1, QFZ93540.1, BAM84831.1, BAD73954.1, XP_001351122.1, BAM84814.1, AAW78190.1, AAW78206.1, UFQ12085.1, AAA29527.1, QFZ93502.1, UFQ12084.1, UFQ12082.1, QFZ93564.1, QFZ93554.1, AAW78200.1, AAF03135.1, AAW78193.1, ACO49498.1, AAN87576.1, ACO49324.1, BAM84859.1, AAN87615.1, ABF66070.1, BAM84771.1, ABF66086.1, QOW08441.1, QFZ93522.1, ABF66066.1, AAW78195.1, ABF66075.1, BAM84762.1, AAN87618.1, AAN87581.1, QOW08394.1, AAN87600.1, AAW78212.1, AW78211.1, AAN87622.1, ACO49332.1, ACO49330.1, KNG78490.1, AAN87620.1, BAD08405.1, QOW08419.1, ACO49375.1, AVC42000.1, P13814.1, ACO49327.1, QOW08472.1, QOW08469.1, AVC41974.1, AAA29547.1, BAM84971.1, ABF66089.1, ACO49331.1, AVC42006.1, ABF83989.1, BAM84962.1, ACO49325.1, ACO49339.1, AVC41971.1, AAA29543.1, AAA63422.1, ACO49409.1, BAM84856.1, ABF83997.1, AVC41992.1, ACO49410.1, or ABF83986.1.

In an aspect of a disclosed mRNA vaccine, a disclosed encoded PfCSP can comprise a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in GenBank Accession No. QOW08375.1, QOW08406.1, AAW78209.1, UFQ12080.1, QFZ93540.1, BAM84831.1, BAD73954.1, XP_001351122.1, BAM84814.1, AAW78190.1, AAW78206.1, UFQ12085.1, AAA29527.1, QFZ93502.1, UFQ12084.1, UFQ12082.1, QFZ93564.1, QFZ93554.1, AAW78200.1, AAF03135.1, AAW78193.1, ACO49498.1, AAN87576.1, ACO49324.1, BAM84859.1, AAN87615.1, ABF66070.1, BAM84771.1, ABF66086.1, QOW08441.1, QFZ93522.1, ABF66066.1, AAW78195.1, ABF66075.1, BAM84762.1, AAN87618.1, AAN87581.1, QOW08394.1, AAN87600.1, AAW78212.1, AW78211.1, AAN87622.1, ACO49332.1, ACO49330.1, KNG78490.1, AAN87620.1, BAD08405.1, QOW08419.1, ACO49375.1, AVC42000.1, P13814.1, ACO49327.1, QOW08472.1, QOW08469.1, AVC41974.1, AAA29547.1, BAM84971.1, ABF66089.1, ACO49331.1, AVC42006.1, ABF83989.1, BAM84962.1, ACO49325.1, ACO49339.1, AVC41971.1, AAA29543.1, AAA63422.1, ACO49409.1, BAM84856.1, ABF83997.1, AVC41992.1, ACO49410.1, or ABF83986.1.

In an aspect of a disclosed mRNA vaccine, a disclosed coding region can comprise an open reading frame. In an aspect, a disclosed open reading frame can encode the antigenic peptide or protein having the sequence set forth in SEQ ID NO:01 or SEQ ID NO:07. In an aspect, a disclosed open reading frame can encode the antigenic peptide or protein having the sequence set forth in SEQ ID NO:02 or SEQ ID NO:08.

In an aspect of a disclosed mRNA vaccine, a disclosed open reading frame can encode Pfs25 or PfCSP. In an aspect, a disclosed open reading frame can encode Pfs25 and PfCSP.

In an aspect of a disclosed mRNA vaccine, a disclosed open reading frame can comprise the sequence set forth in SEQ ID NO:09. In an aspect, a disclosed open reading frame can comprise a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:09. In an aspect, a disclosed open reading frame can comprise the sequence set forth in SEQ ID NO:10. In an aspect, a disclosed open reading frame can comprise a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:10.

Disclosed herein is an mRNA vaccine comprising a RNA molecule comprising (i) a first coding region comprising a first open reading frame, and (ii) a second coding region comprising a second open reading frame.

In an aspect of a disclosed mRNA vaccine, a disclosed first coding region can comprise the sequence set forth in SEQ ID NO:09, and wherein the second coding region comprises the sequence set forth in SEQ ID NO:10.

Disclosed herein is an mRNA vaccine comprising a mRNA molecule comprising a coding region, wherein the coding region encodes at least two, three, four, five, six, seven, eight, and more peptides or proteins (or fragment thereof, or derivative thereof), wherein the peptides or proteins are linked using an amino acid linker sequence. In an aspect, a disclosed amino acid linker can comprise a rigid linker, a flexible linker, a cleavable linker (e.g., self-cleaving peptides), or any combination thereof.

In an aspect of a disclosed mRNA vaccine, a disclosed mRNA molecule can be mono-, bi-, or multicistronic. The coding sequences in a bicistronic or multicistronic mRNA molecule can encode distinct peptides or proteins (such as, for example, Pfs25 and PfCSP).

Disclosed herein is an mRNA vaccine comprising a RNA molecule comprising (i) a first coding region encoding the antigenic peptide or protein having the sequence set forth in SEQ ID NO:01 or SEQ ID NO:07, and (ii) a second coding region encoding the antigenic peptide or protein having the sequence set forth in SEQ ID NO:02 or SEQ ID NO:08. In an aspect, a disclosed mRNA molecule can further comprise a N-terminal signal, a C-terminal GPI anchor, or the combination thereof. In an aspect, a disclosed N-terminal signal can comprise the sequence set forth in SEQ ID NO:03, SEQ ID NO:04, a fragment thereof, or a variant thereof. In an aspect, a disclosed C-terminal GPI anchor can comprise the sequence set forth in SEQ ID NO:05, SEQ ID NO:06, a fragment thereof, or a variant thereof. As known to the art, glycosylphosphatidylinositol (GPI) is a lipid anchor for many cell-surface proteins. In an aspect, a disclosed GPI anchor can represent a posttranslational modification of proteins with a glycolipid and is used ubiquitously in eukaryotes.

In an aspect of a disclosed mRNA vaccine, a disclosed RNA molecule can comprise a N-terminal signal having the sequence set forth in SEQ ID NO:03 and a C-terminal GPI anchor having the sequence set forth in SEQ ID NO:05. In an aspect, a disclosed RNA molecule can comprise a N-terminal signal having the sequence set forth in SEQ ID NO:04 and a C-terminal GPI anchor having the sequence set forth in SEQ ID NO:06. In an aspect, a disclosed RNA molecule can comprise an encoded Pfs25 having the sequence set forth in SEQ ID NO:01, a N-terminal signal having the sequence set forth in SEQ ID NO:03, and a C-terminal GPI anchor having the sequence set forth in SEQ ID NO:05. In an aspect, a disclosed RNA molecule can comprise an encoded PfCSP having the sequence set forth in SEQ ID NO:02, a N-terminal signal having the sequence set forth in SEQ ID NO:04, and a C-terminal GPI anchor having the sequence set forth in SEQ ID NO:06.

In an aspect, a disclosed RNA molecule can comprise one or more modified nucleosides. In an aspect, a modified nucleoside side can comprise a pseudouridine such as, for example, N1-methylpseudouridine-5′-triphosphate. Pseudouridines are known to the skilled person in the art. In an aspect, a disclosed RNA molecule can comprise a partial replacement or a complete replacement of native uridine with modified uridine.

In an aspect of a disclosed mRNA molecule can comprise one or more encoding regions comprising the sequence set forth in SEQ ID NO:07, a fragment thereof, or a variant thereof. In an aspect of a disclosed mRNA molecule can comprise one or more encoding regions comprising the sequence set forth in SEQ ID NO:08, a fragment thereof, or a variant thereof.

In an aspect of a disclosed mRNA vaccine, a disclosed mRNA molecule can comprise one or more mutations at one or more N-linked glycosylation site. In an aspect of a disclosed mRNA molecule can further comprise at least one 5′-cap or 5′-cap structure, a 5′-UTR, a 3′-UTR, a poly-A region, or any combination thereof.

In an aspect, a disclosed 5′-cap or 5′-cap structure can comprise glyceryl, inverted deoxy abasic residue (moiety), 5 methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′ phosphorothioate, phosphorodithioate, or bridging or non-bridging methylphosphonate moiety. In an aspect, a disclosed 5′-cap or 5′-cap structure can comprise CAP1 (additional methylation of the ribose of the adjacent nucleotide of m7GpppN), CAP2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse CAP analogue), modified ARCA (e.g. phosphothioate modified ARCA), inosine, Nl-methyl-guanosine, 2-fluoro-guanosine, 7-deaza-guanosine, 8-oxo-guanosine, 2-amino-guanosine, LNA-guanosine, or 2-azido-guanosine.

In an aspect, a disclosed polyA region can comprise at least 50 nucleotides, at least 60 nucleotides, at least 70 nucleotides, at least 80 nucleotides, at least 90 nucleotides, at least 100 nucleotides, at least 110 nucleotides, at least 120 nucleotides, or more than 120 nucleotides.

In an aspect, a disclosed 5′-UTR can comprise a Kozak sequence. In an aspect, a disclosed the 5′-UTR does not comprise a Kozak sequence. In an aspect, a disclosed 5′-UTR can comprise at least element that provides a translation regulatory activity. In an aspect, a disclosed translational regulatory activity can comprise enhancing and/or improving the translational fidelity of mRNA translation, increasing and/or improving the residence time of the 43 S pre-initiation complex (PIC) or ribosome at, or proximal to, the initiation codon, increasing and/or improving the initiation of polypeptide synthesis at or from the initiation codon, increasing and/or improving the amount of polypeptide translated from the full open reading frame, increasing and/or improving the fidelity of initiation codon decoding by the PIC or ribosome, inhibiting and/or reducing leaky scanning by the PIC or ribosome, inhibiting and/or reducing the rate of decoding the initiation codon by the PIC or ribosome, inhibiting and/or reducing the initiation of polypeptide synthesis at any codon within the mRNA other than the initiation codon, inhibiting and/or reducing the amount of polypeptide translated from any open reading frame within the mRNA other than the full open reading frame, inhibiting and/or reducing the production of aberrant translation products, or any combination thereof.

In an aspect of a disclosed mRNA vaccine, a disclosed mRNA molecule can further comprise one or more stabilizing elements. In an aspect, a disclosed stabilizing element can include, for example, a histone stem-loop. For example, a stem-loop binding protein (SLBP), a 32 kDa protein, is associated with the histone stem-loop at the 3′-end of the histone messages in both the nucleus and the cytoplasm. Its expression level is regulated by the cell cycle; it is peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm.

In an aspect, a disclosed mRNA molecule can comprise an enhancer sequence and/or a promoter sequence, which can be modified or can be unmodified or which can be activated or can be inactivated.

In an aspect, a disclosed mRNA molecule can have one or more AU-rich sequences removed. In an aspect, these one or more AU-rich sequences (e.g., AURES) can be destabilizing sequences and/or can be found in the 3′-UTR. In an aspect, AURES can be removed from a disclosed mRNA molecule. In an aspect, AURES can remain in a disclosed mRNA molecule.

In an aspect of a disclosed mRNA vaccine, a disclosed mRNA molecule can be encapsulated in a lipid nanoparticle (LNP). In an aspect, a disclosed lipid nanoparticles can be prepared from materials comprising ionizable cationic lipids, phospholipid helper lipids, steroidal lipids, neutral phospholipids, phospholipid polyethylene glycol derivatives, or any combination thereof. LNPs can include any cationic lipid suitable for forming a lipid nanoparticle. In an aspect, a disclosed cationic lipid can carry a net positive charge at about physiological pH. In an aspect, a disclosed cationic lipid can be an amino lipid. As used herein, the term “amino lipid” is meant to include those lipids having one or two fatty acid or fatty alkyl chains and an amino head group (including an alkylamino or dialkylamino group) that may be protonated to form a cationic lipid at physiological pH.

Cationic lipids (including amino lipids) are known to the art and are discussed supra. Neutral phospholipids are known to the art and are discussed supra. Steroidal lipids are known to the art and are discussed supra.

In an aspect of a disclosed mRNA vaccine, a disclosed lipid nanoparticle can be a commercially available lipid nanoparticle. In an aspect, for example, a disclosed nanoparticle can comprise a lipid nanoparticle disclosed in U.S. patent Ser. No. 10/221,127, which is incorporated by reference in its entirety for teachings of lipid nanoparticles or lipid nanoparticle formulations for delivery of a disclosed mRNA molecule. The particle size and polydispersity index of disclosed lipid nanoparticles are discussed supra.

In an aspect of a disclosed mRNA vaccine, a disclosed mRNA molecule can comprise a sequence that is CpG depleted and/or codon-optimized for expression in a human cell. In an aspect, “CpG-free” can mean completely free of CpGs or partially free of CpGs. In an aspect, “CpG-free” can mean “CpG-depleted”. In an aspect, “CpG-depleted” can mean “CpG-free”. In an aspect, “CpG-depleted” can mean completely depleted of CpGs or partially depleted of CpGs. In an aspect, “CpG-free” can mean “CpG-optimized” for a desired and/or ideal expression level. CpG depletion and/or optimization is known to the skilled person in the art.

In an aspect of a disclosed mRNA vaccine, a disclosed mRNA molecule encoding at least one antigenic peptide or protein can be codon-optimized. In an aspect, a disclosed sequence encoding Pfs25 and/or PFCSP can be codon-optimized.

In an aspect, a disclosed mRNA vaccine can be prepared by any method known or hereafter developed in the art of pharmacology. In an aspect, for example, a disclosed mRNA vaccine can include the step of bringing the active ingredient (e.g., a disclosed mRNA molecule encoding Pfs25 and/or PfCSP) into association with one or more pharmaceutically acceptable carriers or pharmaceutically acceptable excipients or with one or more other accessory ingredients. In an aspect, a disclosed mRNA vaccine can then be divided, shaped, and/or packaged as desired into a single dose unit or a multi-dose unit.

In an aspect, a disclosed mRNA vaccine can be formulated using one or more disclosed pharmaceutically acceptable carriers or pharmaceutically acceptable excipients to, for example, increase stability, increase cell transfection, permit the sustained or delayed release (e.g., from a depot formulation), alter the biodistribution (e.g., target to specific tissues or cell types), increase the translation of encoded protein (e.g., Pfs25 and/or PfCSP) in vivo, alter the release profile of encoded protein (antigen) in vivo, or any combination thereof.

In an aspect, a disclosed mRNA vaccine can be formulated for extended release of the disclosed mRNA contained therein. In an aspect, an extended-release composition can be conveniently administered to a subject at extended dosing intervals.

In an aspect, a disclosed mRNA vaccine can be formulated for depot administration (e.g., intramuscularly, subcutaneously, intravitreally) to either deliver or release therapeutic agent (e.g., a disclosed mRNA) over extended periods of time.

In an aspect, a disclosed mRNA vaccine can be a liquid formulation.

In an aspect, a disclosed mRNA vaccine can be a lyophilized powder. In an aspect, a disclosed lyophilized powder can be reconstituted prior to administration or can be reconstituted in vivo. In an aspect, for example, a disclosed lyophilized pharmaceutical composition can be formulated in an appropriate dosage form (e.g., an intradermal dosage form such as a disk, rod, or membrane) and can be administered such that the dosage form is rehydrated over time in vivo by the subject's bodily fluids.

In an aspect, a disclosed mRNA vaccine can be an oral formulation, an intramuscular formation, an intravenous formulation, or an inhalation formulation. In an aspect, a disclosed mRNA vaccine can be formulated for an aerosolized inhaler or a dry powder inhaler.

In an aspect, a disclosed mRNA vaccine can induce an immune induction in a subject, can provoke inflammatory response, or a combination thereof. In an aspect, a disclosed mRNA vaccine can be used treat and/or prevent malaria. In an aspect, a disclosed mRNA vaccine can be used treat and/or prevent malaria transmission.

In an aspect, the expression of the encoded Pfs25 antigenic peptide or protein and/or PfCSP antigenic peptide or protein can be sufficient to vaccinate the subject.

In an aspect, an effective amount of a disclosed mRNA vaccine can be sufficient to produce an antigen specific immune response in a subject (e.g., production of antibodies specific to an anti-Pfs25 antigenic peptide or protein, an anti-PfCSP antigenic peptide or protein, or both).

In an aspect, an antigen-specific immune response can be characterized by measuring an anti-Pfs25 and/or PfCSP antigenic peptide or protein antibody titer produced in a subject administered a disclosed mRNA vaccine.

An antibody titer can be a measurement of the amount of antibodies in a subject, for example, antibodies that are specific to a particular antigen (e.g., an anti-Pfs25 and/or PfCSP antigenic peptide or protein) or epitope of an antigen (e.g., an anti-Pfs25 and/or PfCSP antigenic peptide or protein). Antibody titer is typically expressed as the inverse of the greatest dilution that provides a positive result. Enzyme-linked immunosorbent assay (ELISA) is a common assay for determining antibody titers, for example.

In an aspect, an antibody titer can be used to assess whether a subject has had an infection or to determine whether immunizations are required. In an aspect, an antibody titer can be used to determine the strength of an autoimmune response, to determine whether a booster immunization is needed, to determine whether a previous vaccine was effective, to identify any recent or prior infections, or any combination thereof. In an aspect, an antibody titer can be used to determine the strength of an immune response induced in a subject by a disclosed mRNA vaccine (e.g., vaccine encoding a Pfs25 and/or PfCSP antigenic peptide or protein).

In an aspect, a disclosed anti-Pfs25 and/or PfCSP antigenic peptide or protein antibody titer produced in a subject can be increased by at least 1 log relative to a control. For example, an anti-Pfs25 and/or PfCSP antigenic peptide or protein antibody titer produced in a subject can be increased by at least 1.5, at least 2, at least 2.5, or at least 3 log relative to a control. In an aspect, a disclosed anti-Pfs25 and/or PfCSP antigenic peptide or protein antibody titer produced in the subject can be increased by 1, 1.5, 2, 2.5, or 3 log relative to a control. In an aspect, a disclosed anti-Pfs25 and/or PfCSP antigenic peptide or protein antibody titer produced in the subject can be increased by 1-3 log relative to a control. For example, the anti-Pfs25 and/or PfCSP antigenic peptide or protein antibody titer produced in a subject can be increased by 1-1.5, 1-2, 1-2.5, 1-3, 1.5-2, 1.5-2.5, 1.5-3, 2-2.5, 2-3, or 2.5-3 log relative to a control. In an aspect, a disclosed anti-Pfs25 and/or PfCSP antigenic peptide or protein antibody titer produced in a subject can be increased at least 2 times, at least 3 times, at least 4 times, at least 5 times, at least 6 times, at least 7 times, at least 8 times, at least 9 times, at least 10 times, or more than 10 times relative to a control.

In an aspect, a disclosed control can be the anti-Pfs25 and/or PfCSP antigenic peptide or protein antibody titer produced in a subject who has not been administered a disclosed mRNA vaccine. In an aspect, a disclosed control can be the anti-Pfs25 and/or PfCSP antigenic peptide or protein antibody titer in a subject prior to receiving a disclosed mRNA vaccine. In other words, in an aspect, a subject's anti-Pfs25 and/or PfCSP antigenic peptide or protein antibody titer prior to receiving a disclosed mRNA vaccine can serve as a control to the subject's anti-Pfs25 and/or PfCSP antigenic peptide or protein antibody titer after receiving a disclosed mRNA vaccine one or more times.

In an aspect, a disclosed mRNA vaccine can be a transmission-blocking vaccine.

In an aspect, a disclosed mRNA vaccine can comprise a mRNA molecule encoding a homologue of Pfs25 and/or PfCSP, both of which are discussed supra.

### 5. Host Cells

Disclosed herein is a host cell or a host cell line comprising a disclosed mRNA molecule. Disclosed herein is a host cell or a host cell line comprising a disclosed mRNA vaccine.

Disclosed herein is a host cell or a host cell line comprising a mRNA molecule comprising one or more coding regions encoding at least one antigenic peptide or protein, wherein the at least one antigenic peptide or protein is present in one or more life cycle stages of a malarial parasite, and a pharmaceutically acceptable carrier.

Disclosed herein is a host cell or a host cell line comprising a messenger RNA (mRNA) molecule comprising one or more coding regions encoding Plasmodium falciparum surface protein Pfs25, and a pharmaceutically acceptable carrier. In an aspect, the disclosed mRNA can be encapsulated in LNPs.

Disclosed herein is a host cell or a host cell line comprising a messenger RNA (mRNA) molecule comprising a coding region encoding Plasmodium falciparum surface protein Pfs25, wherein the encoded Pfs25 comprises the sequence set forth in SEQ ID NO:01 or SEQ ID NO:07, and a pharmaceutically acceptable carrier. In an aspect, the disclosed mRNA can be encapsulated in LNPs.

Disclosed herein is a host cell or a host cell line comprising a messenger RNA (mRNA) molecule comprising one or more coding regions encoding at least Plasmodium falciparum surface protein circumsporozoite protein (PfCSP), and a pharmaceutically acceptable carrier. In an aspect, the disclosed mRNA can be encapsulated in LNPs.

Disclosed herein is a host cell or a host cell line comprising a messenger RNA (mRNA) molecule comprising a coding region encoding Plasmodium falciparum surface protein circumsporozoite protein (PfCSP), wherein the encoded PfCSP comprises the sequence set forth in SEQ ID NO:02 or SEQ ID NO:08, and a pharmaceutically acceptable carrier. In an aspect, the disclosed mRNA can be encapsulated in LNPs.

Disclosed herein is a host cell or a host cell line comprising a messenger RNA (mRNA) molecule comprising a first coding region encoding a first antigenic peptide or protein, and a second coding region encoding a second antigenic peptide or protein, wherein the first and second antigenic peptides or proteins are present in one or more life cycle stages of a malarial parasite, and a pharmaceutically acceptable carrier. In an aspect, the disclosed mRNA can be encapsulated in LNPs.

Disclosed herein is a host cell or a host cell line comprising a messenger RNA (mRNA) molecule comprising a first coding region encoding comprises Plasmodium falciparum surface protein Pfs25, and a second coding region encoding Plasmodium falciparum surface protein circumsporozoite protein (PfCSP), and a pharmaceutically acceptable carrier. In an aspect, the disclosed mRNA can be encapsulated in LNPs.

Disclosed herein is a host cell or a host cell line comprising a messenger RNA (mRNA-) molecule comprising a first coding region encoding comprises Plasmodium falciparum surface protein Pfs25, and a second coding region encoding Plasmodium falciparum surface protein circumsporozoite protein (PfCSP), wherein the encoded Pfs25 comprises the sequence set forth in SEQ ID NO:01 or SEQ ID NO:07, and wherein the encoded PfCSP comprises the sequence set forth in SEQ ID NO:02 or SEQ ID NO:08, and a pharmaceutically acceptable carrier. In an aspect, the disclosed mRNA can be encapsulated in LNPs.

Disclosed herein is a host cell or a host cell line comprising a mRNA molecule comprising a coding region, wherein the coding region encodes at least two, three, four, five, six, seven, eight, and more peptides or proteins (or fragment thereof, or derivative thereof), wherein the peptides or proteins are linked using an amino acid linker sequence, and a pharmaceutically acceptable carrier. In an aspect, the disclosed mRNA can be encapsulated in LNPs.

Disclosed herein is a host cell or a host cell line comprising a RNA molecule comprising (i) a first coding region encoding the antigenic peptide or protein having the sequence set forth in SEQ ID NO:01 or SEQ ID NO:07, and (ii) a second coding region encoding the antigenic peptide or protein having the sequence set forth in SEQ ID NO:02 or SEQ ID NO:08, and a pharmaceutically acceptable carrier. In an aspect, the disclosed mRNA can be encapsulated in LNPs.

In an aspect, a disclosed host cell or disclosed host cell line can comprise a mammalian or a non-mammalian cell. In an aspect, a disclosed host cell or disclosed host cell line can be transfected by a disclosed plasmid. In an aspect, a disclosed host cell or disclosed host cell line can be transfected by a plasmid comprising the sequence set forth in SEQ ID NO:11 or SEQ ID NO:12.

In an aspect, a disclosed host cell or disclosed host cell line can be transfected by a disclosed mRNA molecule encoding a homologue of Pfs25 and/or PfCSP. In an aspect, for example, a homologue can be Pvs25 (Plasmodium vivax) or can be PfCSP (Plasmodium vivax), both of which are discussed supra.

### 6. Plasmids

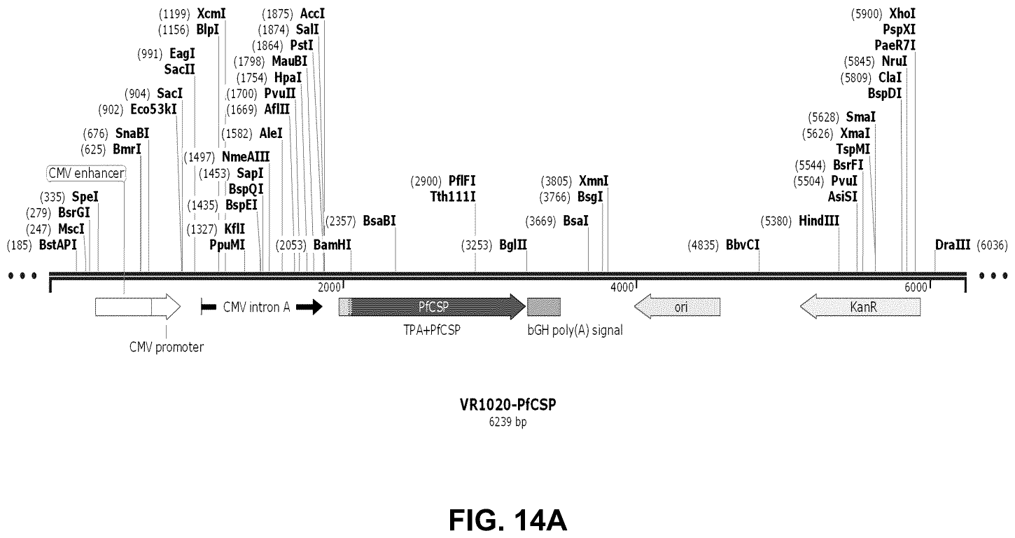

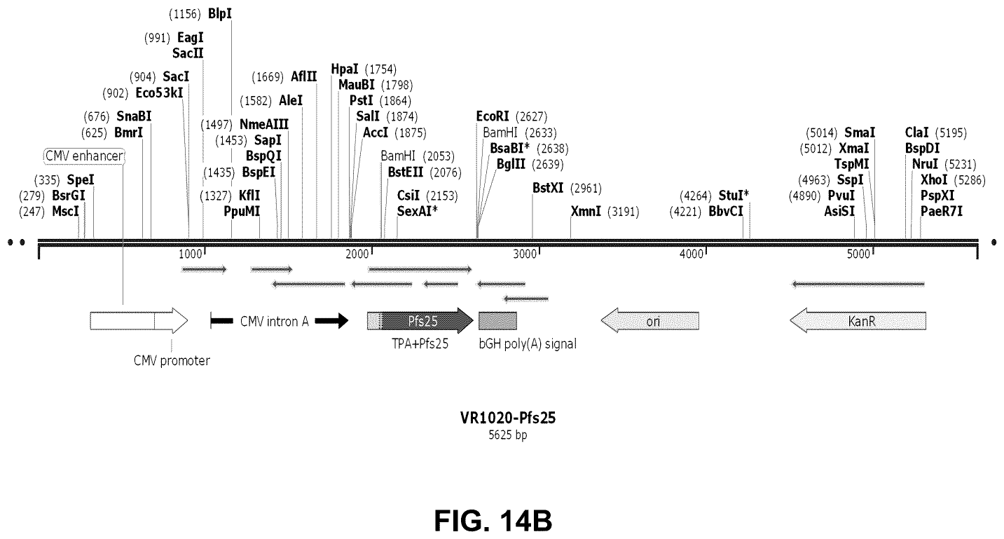

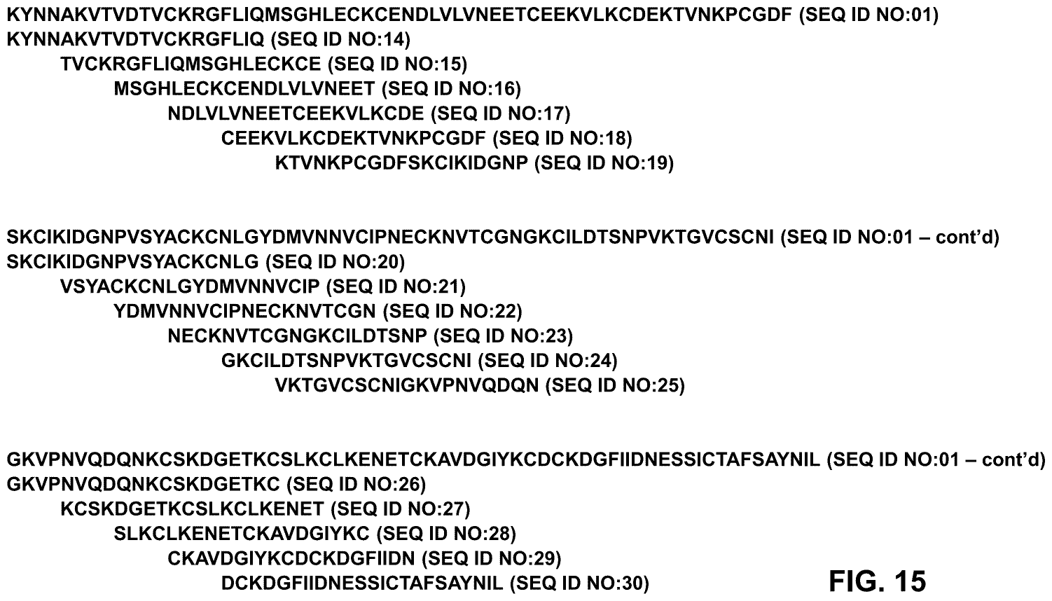

Disclosed herein is a plasmid comprising the sequence set forth in any of SEQ ID NO:10 or SEQ ID NO:11. Plasmids disclosed herein include but are not limited to those listed below.

| Identifier | Plasmid Description |
|---|---|
| 11 | VR1020 comprising PfCSP (FIG. 14A) |
| 12 | VR1020 comprising Pfs25 (FIG. 14B) |

### F. Methods of Inducing an Antigen Specific Immune Response

Disclosed herein is a method of inducing an antigen specific immune response, the method comprising administering to a subject in need thereof a disclosed mRNA vaccine in an amount effective to produce an antigen specific immune response in the subject.

Disclosed herein is a method of inducing an antigen specific immune response, the method comprising administering to a subject in need thereof an mRNA vaccine in an amount effective to produce an antigen specific immune response in the subject, wherein the immune response is specific to the peptide or protein encoded by the mRNA vaccine.

Disclosed herein is a method of inducing an antigen specific immune response, the method comprising administering to a subject a single disclosed mRNA vaccine or administering to a subject more than one disclosed mRNA vaccine in an amount effective to produce an antigen specific immune response in the subject, wherein the immune response is specific to the peptide or protein encoded by the mRNA vaccine.

Disclosed herein is a method of inducing an antigen specific immune response, the method comprising administering to a subject in need thereof a therapeutically effective an mRNA vaccine, wherein the mRNA vaccine comprises at least one messenger ribonucleic acid (mRNA) encoding at least two antigenic peptides or proteins, wherein the at least two antigenic peptides or proteins are present in one or more life cycle stages of a malarial parasite, wherein the immune response is specific to the at least two antigenic peptides or proteins.

Disclosed herein is a method of inducing an antigen specific immune response, the method comprising administering to a subject in need thereof a therapeutically effective an mRNA vaccine, wherein the mRNA vaccine comprises at least one messenger ribonucleic acid (mRNA) encoding at least two antigenic peptides or proteins, wherein the at least two antigenic peptides or proteins are present in one or more life cycle stages of a malarial parasite, wherein the disclosed mRNA molecule is encapsulated in a lipid nanoparticle, wherein the immune response is specific to the at least two antigenic peptides or proteins.

Disclosed herein is a method of inducing an antigen specific immune response, the method comprising administering to a subject in need thereof a therapeutically effective an mRNA vaccine, wherein the mRNA vaccine comprises at least one messenger ribonucleic acid (mRNA) encoding at least two antigenic peptides or proteins, wherein the at least two antigenic peptides or proteins are present in one or more life cycle stages of a malarial parasite, wherein the mRNA vaccine comprises lipid nanoparticles (LNPs), and wherein the mRNA molecule is encapsulated in the LNPs, wherein the immune response is specific to the at least two antigenic peptides or proteins.

Disclosed herein is a method of inducing an antigen specific immune response, the method comprising administering to a subject in need thereof a therapeutically effective an mRNA vaccine, wherein the mRNA vaccine comprises at least one messenger ribonucleic acid (mRNA) encoding at least two antigenic peptides or proteins, wherein the at least two antigenic peptides or proteins are present in one or more life cycle stages of a malarial parasite, wherein the mRNA molecule is encapsulated in a lipid nanoparticle, wherein the mRNA vaccine comprises lipid nanoparticles (LNPs), and wherein the mRNA molecule is encapsulated in the LNPs, wherein the immune response is specific to the at least two antigenic peptides or proteins.

Disclosed herein is a method of inducing an antigen specific immune response, the method comprising administering to a subject in need thereof a therapeutically effective an mRNA vaccine, wherein the mRNA vaccine comprises a messenger RNA (mRNA) molecule, comprising: a first coding region encoding a first antigenic peptide or protein, and a second coding region encoding a second antigenic peptide or protein, wherein the first and second antigenic peptides or proteins are present in one or more life cycle stages of a malarial parasite, wherein the immune response is specific to the first antigenic peptide or protein and to the second first antigenic peptide or protein.

Disclosed herein is a method of inducing an antigen specific immune response, the method comprising administering to a subject in need thereof a therapeutically effective a first mRNA vaccine, wherein the first mRNA vaccine comprises a messenger ribonucleic acid (mRNA) encoding a first antigenic peptide or protein, wherein the first antigenic peptide or protein is present in one or more life cycle stages of a malarial parasite, wherein the mRNA molecule is encapsulated in a lipid nanoparticle, and administering to the subject a therapeutically effective a second mRNA vaccine, wherein the second mRNA vaccine comprises a messenger ribonucleic acid (mRNA) encoding a second antigenic peptide or protein, wherein the second antigenic peptide or protein is present in one or more life cycle stages of a malarial parasite, wherein the mRNA molecule is encapsulated in a lipid nanoparticle, wherein the immune response is specific to the first antigenic peptide or protein and specific to the second antigenic peptide or protein.

Disclosed herein is a method of inducing an antigen specific immune response, the method comprising administering to a subject in need thereof a therapeutically effective a first mRNA vaccine, wherein the first mRNA vaccine comprises a messenger ribonucleic acid (mRNA) encoding Plasmodium falciparum surface protein Pfs25, wherein the mRNA molecule is encapsulated in a lipid nanoparticle, and administering to the subject a therapeutically effective a second mRNA vaccine, wherein the second mRNA vaccine comprises a messenger ribonucleic acid (mRNA) encoding Plasmodium falciparum surface protein circumsporozoite protein (PfCSP), wherein the mRNA molecule is encapsulated in a lipid nanoparticle, wherein the immune response is specific to Pfs25 and PfCSP.

Disclosed herein is a method of inducing an antigen specific immune response, the method comprising administering to a subject in need thereof a therapeutically effective a first mRNA vaccine, wherein the first mRNA vaccine comprises a messenger ribonucleic acid (mRNA) encoding Plasmodium falciparum surface protein Pfs25, wherein the encoded Pfs25 comprises the sequence set forth in SEQ ID NO:01 or SEQ ID NO:07, wherein the mRNA molecule is encapsulated in a lipid nanoparticle, and administering to the subject a therapeutically effective a second mRNA vaccine, wherein the second mRNA vaccine comprises a messenger ribonucleic acid (mRNA) encoding Plasmodium falciparum surface protein circumsporozoite protein (PfCSP), wherein the encoded PfCSP comprises the sequence set forth in SEQ ID NO:02 or SEQ ID NO:08, wherein the mRNA molecule is encapsulated in a lipid nanoparticle, wherein the immune response is specific to Pfs25 and PfCSP.

Disclosed herein is a method of inducing an antigen specific immune response, the method comprising administering to a subject in need thereof a therapeutically effective a first mRNA vaccine, wherein the first mRNA vaccine comprises a messenger ribonucleic acid (mRNA) encoding Plasmodium falciparum surface protein Pfs25, wherein the encoded Pfs25 comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:01 or SEQ ID NO:07, wherein the mRNA molecule is encapsulated in a lipid nanoparticle, and administering to the subject a therapeutically effective a second mRNA vaccine, wherein the second mRNA vaccine comprises a messenger ribonucleic acid (mRNA) encoding Plasmodium falciparum surface protein circumsporozoite protein (PfCSP), wherein the encoded PfCSP comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:02 or SEQ ID NO:08, wherein the mRNA molecule is encapsulated in a lipid nanoparticle, wherein the immune response is specific to Pfs25 and PfCSP.

Disclosed herein is a method of inducing an antigen specific immune response, the method comprising administering to a subject in need thereof a therapeutically effective a first mRNA vaccine, wherein the first mRNA vaccine comprises a messenger ribonucleic acid (mRNA) encoding Plasmodium falciparum surface protein Pfs25, wherein the encoded Pfs25 comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in GenBank Accession No. XP_001347587.1, EUT85305.1, AAD39544.1, UFQ05488.1, UFQ05499.1, ETW42495.1, QOQ86355.1, QRV62119.1, QRV62061.1, QOQ86414.1, QOQ86416.1, QRV62092.1, 6PHB_E, 6AZZ_A, AIV43628.1, AIV43629.1, AIV43624.1, AIV43630.1, AIV43626.1, AIV43634.1, ASU08938.1, AKJ86874.1, AIV43627.1, ASU08940.1, ASU08939.1, ALF99893.1, ASU09129.1, ASU09128.1, ASU09130.1, UIT08865.1, UIT08866.1, ALF99891.1, ALF99892.1, AIV43625.1, ALF99890.1, or AKJ86873.1, wherein the mRNA molecule is encapsulated in a lipid nanoparticle, and administering to the subject a therapeutically effective a second mRNA vaccine, wherein the second mRNA vaccine comprises a messenger ribonucleic acid (mRNA) encoding Plasmodium falciparum surface protein circumsporozoite protein (PfCSP), wherein the encoded PfCSP comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in GenBank Accession No. QOW08375.1, QOW08406.1, AAW78209.1, UFQ12080.1, QFZ93540.1, BAM84831.1, BAD73954.1, XP_001351122.1, BAM84814.1, AAW78190.1, AAW78206.1, UFQ12085.1, AAA29527.1, QFZ93502.1, UFQ12084.1, UFQ12082.1, QFZ93564.1, QFZ93554.1, AAW78200.1, AAF03135.1, AAW78193.1, ACO49498.1, AAN87576.1, ACO49324.1, BAM84859.1, AAN87615.1, ABF66070.1, BAM84771.1, ABF66086.1, QOW08441.1, QFZ93522.1, ABF66066.1, AAW78195.1, ABF66075.1, BAM84762.1, AAN87618.1, AAN87581.1, QOW08394.1, AAN87600.1, AAW78212.1, AW78211.1, AAN87622.1, ACO49332.1, ACO49330.1, KNG78490.1, AAN87620.1, BAD08405.1, QOW08419.1, ACO49375.1, AVC42000.1, P13814.1, ACO49327.1, QOW08472.1, QOW08469.1, AVC41974.1, AAA29547.1, BAM84971.1, ABF66089.1, ACO49331.1, AVC42006.1, ABF83989.1, BAM84962.1, ACO49325.1, ACO49339.1, AVC41971.1, AAA29543.1, AAA63422.1, ACO49409.1, BAM84856.1, ABF83997.1, AVC41992.1, ACO49410.1, or ABF83986.1, wherein the mRNA molecule is encapsulated in a lipid nanoparticle, wherein the immune response is specific to Pfs25 and PfCSP.

Disclosed herein is a method of inducing an antigen specific immune response, the method comprising administering to a subject in need thereof a therapeutically effective a first mRNA vaccine, wherein the first mRNA vaccine comprises a messenger ribonucleic acid (mRNA) encoding Plasmodium falciparum surface protein Pfs25, wherein the encoded Pfs25 comprises the sequence set forth in GenBank Accession No. XP_001347587.1, EUT85305.1, AAD39544.1, UFQ05488.1, UFQ05499.1, ETW42495.1, QOQ86355.1, QRV62119.1, QRV62061.1, QOQ86414.1, QOQ86416.1, QRV62092.1, 6PHB_E, 6AZZ_A, AIV43628.1, AIV43629.1, AIV43624.1, AIV43630.1, AIV43626.1, AIV43634.1, ASU08938.1, AKJ86874.1, AIV43627.1, ASU08940.1, ASU08939.1, ALF99893.1, ASU09129.1, ASU09128.1, ASU09130.1, UIT08865.1, UIT08866.1, ALF99891.1, ALF99892.1, AIV43625.1, ALF99890.1, or AKJ86873.1, wherein the mRNA molecule is encapsulated in a lipid nanoparticle, and administering to the subject a therapeutically effective a second mRNA vaccine, wherein the second mRNA vaccine comprises a messenger ribonucleic acid (mRNA) encoding Plasmodium falciparum surface protein circumsporozoite protein (PfCSP), wherein the encoded PfCSP comprises the sequence set forth in GenBank Accession No. QOW08375.1, QOW08406.1, AAW78209.1, UFQ12080.1, QFZ93540.1, BAM84831.1, BAD73954.1, XP_001351122.1, BAM84814.1, AAW78190.1, AAW78206.1, UFQ12085.1, AAA29527.1, QFZ93502.1, UFQ12084.1, UFQ12082.1, QFZ93564.1, QFZ93554.1, AAW78200.1, AAF03135.1, AAW78193.1, ACO49498.1, AAN87576.1, ACO49324.1, BAM84859.1, AAN87615.1, ABF66070.1, BAM84771.1, ABF66086.1, QOW08441.1, QFZ93522.1, ABF66066.1, AAW78195.1, ABF66075.1, BAM84762.1, AAN87618.1, AAN87581.1, QOW08394.1, AAN87600.1, AAW78212.1, AW78211.1, AAN87622.1, ACO49332.1, ACO49330.1, KNG78490.1, AAN87620.1, BAD08405.1, QOW08419.1, ACO49375.1, AVC42000.1, P13814.1, ACO49327.1, QOW08472.1, QOW08469.1, AVC41974.1, AAA29547.1, BAM84971.1, ABF66089.1, ACO49331.1, AVC42006.1, ABF83989.1, BAM84962.1, ACO49325.1, ACO49339.1, AVC41971.1, AAA29543.1, AAA63422.1, ACO49409.1, BAM84856.1, ABF83997.1, AVC41992.1, ACO49410.1, or ABF83986.1, wherein the mRNA molecule is encapsulated in a lipid nanoparticle, wherein the immune response is specific to Pfs25 and PfCSP.

Disclosed herein is a method of inducing an antigen specific immune response, the method comprising administering to a subject in need thereof a therapeutically effective a first mRNA vaccine, wherein the first mRNA vaccine comprises a lipid nanoparticle encapsulated messenger ribonucleic acid (mRNA) encoding Plasmodium falciparum surface protein Pfs25, and administering to the subject a therapeutically effective a second mRNA vaccine, wherein the second mRNA vaccine comprises a lipid nanoparticle encapsulated messenger ribonucleic acid (mRNA) encoding Plasmodium falciparum surface protein circumsporozoite protein (PfCSP), wherein the immune response is specific to Pfs25 and PfCSP.

Disclosed herein is a method of inducing an antigen specific immune response, the method comprising administering to a subject in need thereof a therapeutically effective a first mRNA vaccine, wherein the first mRNA vaccine comprises a lipid nanoparticle encapsulated messenger ribonucleic acid (mRNA) encoding a first antigenic peptide or protein, wherein the first antigenic peptide or protein is present in one or more life cycle stages of a malarial parasite, and administering to the subject a therapeutically effective a second mRNA vaccine, wherein the second mRNA vaccine comprises a lipid nanoparticle encapsulated messenger ribonucleic acid (mRNA) encoding a second antigenic peptide or protein, wherein the second antigenic peptide or protein is present in one or more life cycle stages of a malarial parasite, wherein the immune response is specific to the first antigenic peptide or protein and specific to the second antigenic peptide or protein.

Disclosed herein is a method of inducing an antigen specific immune response, the method comprising administering to a subject in need thereof a therapeutically effective a first mRNA vaccine, wherein the first mRNA vaccine comprises a lipid nanoparticle encapsulated messenger administering to the subject a therapeutically effective a second mRNA vaccine, wherein the second mRNA vaccine comprises a lipid nanoparticle encapsulated messenger ribonucleic acid (mRNA) encoding Plasmodium falciparum surface protein circumsporozoite protein (PfCSP), wherein the immune response is specific to Pfs25 and PfCSP.

Disclosed herein is a method of inducing an antigen specific immune response, the method comprising administering to a subject in need thereof a therapeutically effective a first mRNA vaccine, wherein the first mRNA vaccine comprises a lipid nanoparticle encapsulated messenger ribonucleic acid (mRNA) encoding Plasmodium falciparum surface protein Pfs25, wherein the encoded Pfs25 comprises the sequence set forth in SEQ ID NO:01 or SEQ ID NO:07, and administering to the subject a therapeutically effective a second mRNA vaccine, wherein the second mRNA vaccine comprises a lipid nanoparticle encapsulated messenger ribonucleic acid (mRNA) encoding Plasmodium falciparum surface protein circumsporozoite protein (PfCSP), wherein the encoded PfCSP comprises the sequence set forth in SEQ ID NO:02 or SEQ ID NO:08, wherein the immune response is specific to Pfs25 and PfCSP.

Disclosed herein is a method of inducing an antigen specific immune response, the method comprising administering to a subject in need thereof a therapeutically effective a first mRNA vaccine, wherein the first mRNA vaccine comprises a lipid nanoparticle encapsulated messenger ribonucleic acid (mRNA) encoding Plasmodium falciparum surface protein Pfs25, wherein the encoded Pfs25 comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:01 or SEQ ID NO:07, and administering to the subject a therapeutically effective a second mRNA vaccine, wherein the second mRNA vaccine comprises a lipid nanoparticle encapsulated messenger ribonucleic acid (mRNA) encoding Plasmodium falciparum surface protein circumsporozoite protein (PfCSP), wherein the encoded PfCSP comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:02 or SEQ ID NO:08, wherein the immune response is specific to Pfs25 and PfCSP.

Disclosed herein is a method of inducing an antigen specific immune response, the method comprising administering to a subject in need thereof a therapeutically effective a first mRNA vaccine, wherein the first mRNA vaccine comprises a lipid nanoparticle encapsulated messenger ribonucleic acid (mRNA) encoding Plasmodium falciparum surface protein Pfs25, wherein the encoded Pfs25 comprises the sequence set forth in GenBank Accession No. XP_001347587.1, EUT85305.1, AAD39544.1, UFQ05488.1, UFQ05499.1, ETW42495.1, QOQ86355.1, QRV62119.1, QRV62061.1, QOQ86414.1, QOQ86416.1, QRV62092.1, 6PHB_E, 6AZZ_A, AIV43628.1, AIV43629.1, AIV43624.1, AIV43630.1, AIV43626.1, AIV43634.1, ASU08938.1, AKJ86874.1, AIV43627.1, ASU08940.1, ASU08939.1, ALF99893.1, ASU09129.1, ASU09128.1, ASU09130.1, UIT08865.1, UIT08866.1, ALF99891.1, ALF99892.1, AIV43625.1, ALF99890.1, or AKJ86873.1, and administering to the subject a therapeutically effective a second mRNA vaccine, wherein the second mRNA vaccine comprises a lipid nanoparticle encapsulated messenger ribonucleic acid (mRNA) encoding Plasmodium falciparum surface protein circumsporozoite protein (PfCSP), wherein the encoded PfCSP comprises the sequence set forth in GenBank Accession No. QOW08375.1, QOW08406.1, AAW78209.1, UFQ12080.1, QFZ93540.1, BAM84831.1, BAD73954.1, XP_001351122.1, BAM84814.1, AAW78190.1, AAW78206.1, UFQ12085.1, AAA29527.1, QFZ93502.1, UFQ12084.1, UFQ12082.1, QFZ93564.1, QFZ93554.1, AAW78200.1, AAF03135.1, AAW78193.1, ACO49498.1, AAN87576.1, ACO49324.1, BAM84859.1, AAN87615.1, ABF66070.1, BAM84771.1, ABF66086.1, QOW08441.1, QFZ93522.1, ABF66066.1, AAW78195.1, ABF66075.1, BAM84762.1, AAN87618.1, AAN87581.1, QOW08394.1, AAN87600.1, AAW78212.1, AW78211.1, AAN87622.1, ACO49332.1, ACO49330.1, KNG78490.1, AAN87620.1, BAD08405.1, QOW08419.1, ACO49375.1, AVC42000.1, P13814.1, ACO49327.1, QOW08472.1, QOW08469.1, AVC41974.1, AAA29547.1, BAM84971.1, ABF66089.1, ACO49331.1, AVC42006.1, ABF83989.1, BAM84962.1, ACO49325.1, ACO49339.1, AVC41971.1, AAA29543.1, AAA63422.1, ACO49409.1, BAM84856.1, ABF83997.1, AVC41992.1, ACO49410.1, or ABF83986.1, wherein the immune response is specific to Pfs25 and PfCSP.

Disclosed herein is a method of inducing an antigen specific immune response, the method comprising administering to a subject in need thereof a therapeutically effective a first mRNA vaccine, wherein the first mRNA vaccine comprises a lipid nanoparticle encapsulated messenger ribonucleic acid (mRNA) encoding Plasmodium falciparum surface protein Pfs25, wherein the encoded Pfs25 comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in GenBank Accession No. XP_001347587.1, EUT85305.1, AAD39544.1, UFQ05488.1, UFQ05499.1, ETW42495.1, QOQ86355.1, QRV62119.1, QRV62061.1, QOQ86414.1, QOQ86416.1, QRV62092.1, 6PHB_E, 6AZZ_A, AIV43628.1, AIV43629.1, AIV43624.1, AIV43630.1, AIV43626.1, AIV43634.1, ASU08938.1, AKJ86874.1, AIV43627.1, ASU08940.1, ASU08939.1, ALF99893.1, ASU09129.1, ASU09128.1, ASU09130.1, UIT08865.1, UIT08866.1, ALF99891.1, ALF99892.1, AIV43625.1, ALF99890.1, or AKJ86873.1, and administering to the subject a therapeutically effective a second mRNA vaccine, wherein the second mRNA vaccine comprises a lipid nanoparticle encapsulated messenger ribonucleic acid (mRNA) encoding Plasmodium falciparum surface protein circumsporozoite protein (PfCSP), wherein the encoded PfCSP comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in GenBank Accession No. QOW08375.1, QOW08406.1, AAW78209.1, UFQ12080.1, QFZ93540.1, BAM84831.1, BAD73954.1, XP_001351122.1, BAM84814.1, AAW78190.1, AAW78206.1, UFQ12085.1, AAA29527.1, QFZ93502.1, UFQ12084.1, UFQ12082.1, QFZ93564.1, QFZ93554.1, AAW78200.1, AAF03135.1, AAW78193.1, ACO49498.1, AAN87576.1, ACO49324.1, BAM84859.1, AAN87615.1, ABF66070.1, BAM84771.1, ABF66086.1, QOW08441.1, QFZ93522.1, ABF66066.1, AAW78195.1, ABF66075.1, BAM84762.1, AAN87618.1, AAN87581.1, QOW08394.1, AAN87600.1, AAW78212.1, AW78211.1, AAN87622.1, ACO49332.1, ACO49330.1, KNG78490.1, AAN87620.1, BAD08405.1, QOW08419.1, ACO49375.1, AVC42000.1, P13814.1, ACO49327.1, QOW08472.1, QOW08469.1, AVC41974.1, AAA29547.1, BAM84971.1, ABF66089.1, ACO49331.1, AVC42006.1, ABF83989.1, BAM84962.1, ACO49325.1, ACO49339.1, AVC41971.1, AAA29543.1, AAA63422.1, ACO49409.1, BAM84856.1, ABF83997.1, AVC41992.1, ACO49410.1, or ABF83986.1, wherein the immune response is specific to Pfs25 and PfCSP.

In an aspect, a disclosed method of inducing an antigen specific immune response, can further comprise administering to the subject artesunate, aremether-lumefantrine, clindamycin, doxycycline, atovaquone, chloroquine, mefloquine, quinine, primaquine, or any combination thereof.

In an aspect of a disclosed method of inducing an antigen specific immune response, administering a first mRNA vaccine occurs prior to administering a second mRNA vaccine. In an aspect of a disclosed method of inducing an antigen specific immune response, administering a first mRNA vaccine occurs concurrently with administering a second mRNA vaccine. In an aspect of a disclosed method of inducing an antigen specific immune response, administering a first mRNA vaccine occurs after administering a second mRNA vaccine. In an aspect of a disclosed method of inducing an antigen specific immune response, the schedule for administering the first mRNA vaccine and administering the second mRNA vaccine can be established prior to treatment. In an aspect of a disclosed method of inducing an antigen specific immune response, the schedule for administering the first mRNA vaccine and administering the second mRNA vaccine can be established prior to treatment and subsequently modified.

In an aspect, an effective amount of a disclosed mRNA vaccine can comprise a range from about 0.0005 mg/kg body weight to about 500 mg/kg body weight. In an aspect, a disclosed therapeutically effective dose can range from about 0.001 mg/kg body weight to about 400 mg/kg body weight, from about 0.001 mg/kg body weight to about 300 mg/kg body weight, from about 0.001 mg/kg body weight to about 200 mg/kg body weight, from about 0.001 mg/kg body weight to about 100 mg/kg body weight, from about 0.001 mg/kg body weight to about 90 mg/kg body weight, from about 0.001 kg/kg body weight to about 80 mg/kg body weight, from about 0.001 mg/kg body weight to about 70 mg/kg body weight, from about 0.001 mg/kg body weight to about 60 mg/kg body weight, from about 0.001 mg/kg body weight to about 50 mg/kg body weight, from about 0.001 mg/kg body weight to about 40 mg/kg body weight, from about 0.001 mg/kg body weight to about 30 mg/kg body weight, from about 0.001 mg/kg body weight to about 25 mg/kg body weight, from about 0.001 mg/kg body weight to about 20 mg/kg body weight, from about 0.001 mg/kg body weight to about 15 mg/kg body weight, from about 0.001 mg/kg body weight to about 10 mg/kg body weight, or from about 0.001 mg/kg body weight to about 5 mg/kg body weight.

In an aspect, an effective amount of a disclosed mRNA vaccine can be as low as 10 μg, administered for example as a single dose or as two 5 μg doses. In an aspect, an effective amount of a disclosed mRNA vaccine can comprise a range from about 10 μg to about 300 μg. For example, in an aspect, an effective amount can be a total dose of 10 μg, 20 μg, 25 μg, 30 μg, 35 μg, 40 μg, 45 μg, 50 μg, 55 μg, 60 μg, 65 μg, 70 30 μg, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg or 200 μg, 210 μg, 220 μg, 230 μg, 240 μg, 250 μg, 260 μg, 270 μg, 280 μg, 290 μg or 300 μg.

In an aspect, a disclosed mRNA vaccine can be administered and dosed in accordance with current medical practice, taking into account the clinical condition of the subject, the site and method of administration, the scheduling of administration, the subject's age, sex, body weight and other factors relevant to clinicians of ordinary skill in the art. In an aspect, an effective amount of a disclosed mRNA vaccine can be determined by such relevant considerations as are known to those of ordinary skill in experimental clinical research, pharmacological, clinical, and medical arts.

In an aspect, a subject can be suspected of having or can be diagnosed with having malaria. In an aspect, a disclosed subject can be symptomatic or asymptomatic. In an aspect, a subject can be a subject in need of treatment of malaria.

In an aspect, a disclosed method can further comprise repeating one or more administering steps. In an aspect, a disclosed administering step can be repeated one or more, two or more, three or more, four or more, or more than four times.

In an aspect, a disclosed method can further comprise repeating one or more, two or more, three or more, four or more, or more than four times the administering of a disclosed first mRNA vaccine comprising a messenger ribonucleic acid (mRNA) encoding a disclosed first antigenic peptide or protein. For example, in an aspect, a disclosed method can further comprise repeating one or more, two or more, three or more, four or more, or more than four times the administering of a disclosed second mRNA vaccine comprising a messenger ribonucleic acid (mRNA) encoding a disclosed second antigenic peptide or protein. For example, in an aspect, a disclosed method can further comprise repeating one or more, two or more, three or more, four or more, or more than four times (i) the administering of a disclosed first mRNA vaccine comprising a messenger ribonucleic acid (mRNA) encoding a disclosed first antigenic peptide or protein, and (ii) the administering of a disclosed second mRNA vaccine comprising a messenger ribonucleic acid (mRNA) encoding a disclosed second antigenic peptide or protein.

In an aspect, a disclosed method can further comprise repeating one or more, two or more, three or more, four or more, or more than four times the administering of a disclosed mRNA vaccine comprising a messenger ribonucleic acid (mRNA) encoding Plasmodium falciparum surface protein Pfs25.

In an aspect, a disclosed method can further comprise repeating one or more, two or more, three or more, four or more, or more than four times the administering of a disclosed mRNA vaccine comprising a messenger ribonucleic acid (mRNA) encoding Plasmodium falciparum surface protein circumsporozoite protein (PfCSP).

For example, in an aspect, a disclosed method can further comprise repeating one or more, two or more, three or more, four or more, or more than four times (i) the administering of a disclosed mRNA vaccine comprising a messenger ribonucleic acid (mRNA) encoding Plasmodium falciparum surface protein Pfs25, and (ii) the administering of a disclosed mRNA vaccine comprises a messenger ribonucleic acid (mRNA) encoding Plasmodium falciparum surface protein circumsporozoite protein (PfCSP).

For example, in an aspect, a disclosed method can further comprise repeating one or more, two or more, three or more, four or more, or more than four times the administering of a disclosed mRNA vaccine comprising a messenger ribonucleic acid (mRNA) encoding Plasmodium falciparum surface protein Pfs25 wherein the encoded Pfs25 comprises the sequence set forth in SEQ ID NO:01 or SEQ ID NO:07. For example, in an aspect, a disclosed method can further comprise repeating one or more, two or more, three or more, four or more, or more than four times the administering of a disclosed mRNA vaccine comprising a messenger ribonucleic acid (mRNA) encoding Plasmodium falciparum surface protein circumsporozoite protein (PfCSP), wherein the encoded PfCSP comprises the sequence set forth in SEQ ID NO:02 or SEQ ID NO:08.

For example, in an aspect, a disclosed method can further comprise repeating one or more, two or more, three or more, four or more, or more than four times (i) the administering of a disclosed mRNA vaccine comprising a messenger ribonucleic acid (mRNA) encoding Plasmodium falciparum surface protein Pfs25, wherein the encoded Pfs25 comprises the sequence set forth in SEQ ID NO:01 or SEQ ID NO:07, and (ii) the administering of a disclosed mRNA vaccine comprises a messenger ribonucleic acid (mRNA) encoding Plasmodium falciparum surface protein circumsporozoite protein (PfCSP), wherein the encoded PfCSP comprises the sequence set forth in SEQ ID NO:02 or SEQ ID NO:08.

In an aspect, a disclosed method of inducing an antigen specific immune response can further comprise monitoring the subject for adverse effects. In an aspect, in the absence of adverse effects, the method can further comprise continuing to treat the subject and/or continuing to monitor the subject. In an aspect, in the presence of adverse effects, the method can further comprise modifying the treating step, modifying the administering step, or both. Methods of monitoring a subject's well-being can include both subjective and objective criteria (and are discussed supra). Such methods are known to the skilled person.

In an aspect, a disclosed method can further comprise administering to the subject a therapeutically effective amount of a therapeutic agent. Therapeutic agents are known.

In an aspect, a disclosed method can further comprise administering to the subject a therapeutically effective amount of one or more immune modulators. In an aspect, the one or more immune modulators comprise methotrexate, rituximab, intravenous gamma globulin, Tacrolimus, SVP-Rapamycin, bortezomib, or a combination thereof.

In an aspect, a disclosed method can further comprise validating and/or characterizing a disclosed mRNA vaccine. In an aspect, validating and/or charactering a disclosed mRNA vaccine can comprise determining the function and/or activity of the disclosed mRNA vaccine. In an aspect, validating and/or charactering a disclosed mRNA vaccine can comprise measuring and/or determining the efficacy of the disclosed mRNA vaccine. In an aspect, validating and/or charactering a disclosed mRNA vaccine can comprise measuring and/or determining the transmission blocking ability of the disclosed mRNA vaccine. In an aspect, validating and/or charactering a disclosed mRNA vaccine can comprise measuring and/or determining the transmission reducing activity of the disclosed mRNA vaccine. In an aspect, transmission reducing activity can be defined as the present reduction in mean oocysts between test IgG-fed mosquitoes and control IgG-fed mosquitoes. In an aspect, validating and/or charactering a disclosed mRNA vaccine can comprise measuring and/or determining the transmission blocking activity of the disclosed mRNA vaccine. In an aspect, transmission blocking activity can be defined as the present reduction in the proportion of infected mosquitoes between the test IgG-fed mosquitoes and control IgG-fed mosquitoes. In an aspect, validating and/or characterizing a disclosed mRNA vaccine and/or the function and/or activity of a disclosed mRNA vaccine can comprise using an animal model (such as, for example, mice, rats, hamsters, etc.). In an aspect, validating and/or characterizing a disclosed mRNA vaccine and/or the function and/or activity of a disclosed mRNA vaccine can comprise using a mosquito model.

In an aspect of a disclosed method of inducing an antigen specific immune response, administering a disclosed mRNA vaccine can comprise intravenous administration, intracerebral administration, intra-CSF administration, intracerebroventricular (ICV) administration, intraventricular administration, intra-cisterna magna (ICM) administration, intraparenchymal administration, intrathecal (lumbar, cisternal, or both) administration, intrahepatic administration, hepatic intra-arterial administration, hepatic portal vein (HPV) administration, or any combination thereof.

In an aspect of a disclosed method, a subject can be immunized with a single administration of a disclosed mRNA molecule, a disclosed mRNA vaccine, and/or a disclosed pharmaceutical formulation. In an aspect of a disclosed method, a subject can be immunized with two administrations of a disclosed mRNA molecule, a disclosed mRNA vaccine, and/or a disclosed pharmaceutical formulation. In an aspect of a disclosed method, a subject can be immunized with three administrations of a disclosed mRNA molecule, a disclosed mRNA vaccine, and/or a disclosed pharmaceutical formulation. In an aspect of a disclosed method, a subject can be immunized with four administrations of a disclosed mRNA molecule, a disclosed mRNA vaccine, and/or a disclosed pharmaceutical formulation. In an aspect of a disclosed method, a subject can be immunized with five administrations of a disclosed mRNA molecule, a disclosed mRNA vaccine, and/or a disclosed pharmaceutical formulation. In an aspect of a disclosed method, a subject can be immunized with more than 5 administrations of a disclosed mRNA molecule, a disclosed mRNA vaccine, and/or a disclosed pharmaceutical formulation.

In an aspect, a disclosed method of inducing an antigen specific immune response can employ multiple routes of administration to the subject. In an aspect, a disclosed method can employ a first route of administration that can be the same or different as a second and/or subsequent routes of administration. In an aspect, a disclosed mRNA molecule, a disclosed mRNA vaccine, a disclosed pharmaceutical formulation, or any combination thereof can be concurrently and/or serially administered to a subject via multiple routes of administration. For example, in an aspect, administering a disclosed mRNA molecule, a disclosed mRNA vaccine, and/or a disclosed pharmaceutical formulation can comprise intravenous administration and intra-cistern magna (ICM) administration. In an aspect, administering a disclosed mRNA molecule, a disclosed mRNA vaccine, and/or a disclosed pharmaceutical formulation can comprise IV administration and intrathecal (ITH) administration.

In an aspect, a disclosed method of inducing an antigen specific immune response can further comprise establishing biodistribution, persistence, and integration analysis. In an aspect, a disclosed method of inducing an antigen specific immune response can further comprise performing assays to assess the distribution and duration of the immune response. In an aspect, a disclosed method of inducing an antigen specific immune response can further comprise disrupting and/or interrupting malaria transmission. In an aspect, disrupting and/or interrupting malaria transmission can comprise a partial disruption and/or interruption and/or a full disruption and/or interruption. In an aspect, a disclosed method of inducing an antigen specific immune response can further comprise reducing the pathological phenotype associated with malaria. In an aspect, a disclosed method of inducing an antigen specific immune response can further comprise diagnosing the subject with malaria. In an aspect, a disclosed method of inducing an antigen specific immune response can further comprise disrupting the sexual life cycle of the malarial parasite. In an aspect, a disclosed method of inducing an antigen specific immune response can further comprise causing the cessation and/or preventing and/or slowing the development of the malarial parasite. In an aspect, a disclosed method of inducing an antigen specific immune response can further comprise reducing the transmission of the malarial parasite. In an aspect, a disclosed method of inducing an antigen specific immune response can further comprise slowing malarial disease progression in a subject.

In an aspect, a disclosed method of inducing an antigen specific immune response can further comprise administering to the subject a therapeutically effective amount of an agent that can correct one or more aspects of a dysregulated metabolic or enzymatic pathway.

In an aspect, a disclosed method can further comprise restoring one or more aspects of cellular homeostasis and/or cellular functionality and/or metabolic dysregulation. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of malaria; (vii) reversing, inhibiting, preventing, stabilizing, and/or slowing the rate of progression and/or transmission of malaria, or (viii) any combination thereof. In an aspect, restoring one or more aspects of cellular homeostasis can comprise improving, enhancing, restoring, and/or preserving one or more aspects of cellular structural and/or functional integrity. In an aspect of a disclosed method of inducing an antigen specific immune response, techniques to monitor, measure, and/or assess the restoring one or more aspects of cellular homeostasis and/or cellular functionality can comprise qualitative (or subjective) means as well as quantitative (or objective) means. These means are known to the skilled person. For example, representative regulated variables and sensors relating to systemic homeostasis are discussed supra.

In an aspect, a disclosed method of inducing an antigen specific immune response can further comprise repeating one or more steps of the method and/or modifying one or more steps of the method (such as, for example, an administering step).

In an aspect, a disclosed method of inducing an antigen specific immune response can further comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount of one or more of a disclosed mRNA molecule, a disclosed mRNA vaccine, a disclosed pharmaceutical formulation, or any combination thereof administered to a subject, or by changing the frequency of administration of one or more of a disclosed mRNA molecule, a disclosed mRNA vaccine, a disclosed pharmaceutical formulation, or any combination thereof to a subject, or by changing the duration of time one or more of a disclosed mRNA molecule, a disclosed mRNA vaccine, a disclosed pharmaceutical formulation, or any combination thereof are administered to a subject.

In an aspect, a disclosed method of inducing an antigen specific immune response can further comprise generating and/or validating one or more of the disclosed mRNA molecules, one or more of the disclosed mRNA vaccines, one or more of the disclosed pharmaceutical formulations, or any combination thereof.

In an aspect of a disclosed method of inducing an antigen specific immune response, a disclosed pharmaceutical formulation (such as, for example, a disclosed pharmaceutical formulation comprising a disclosed mRNA vaccine) can be substituted in lieu of a disclosed mRNA vaccine.

In an aspect of a disclosed method of inducing an antigen specific immune response, a disclosed mRNA molecule can encode a homologue of Pfs25 and/or PfCSP. In an aspect, for example, a homologue can be Pvs25 (Plasmodium vivax) or can be PvCSP (Plasmodium vivax), both of which are discussed supra.

### G. Methods of Interrupting Malaria Transmission

Disclosed herein is a method of interrupting malaria transmission, the method comprising administering to a subject in need thereof a disclosed mRNA vaccine in an amount effective to disrupt the sexual life cycle of the malarial parasite.

Disclosed herein is a method of interrupting malaria transmission, the method comprising administering to a subject in need thereof an mRNA vaccine in an amount effective to produce an antigen specific immune response in the subject, wherein the immune response is specific to the peptide or protein encoded by the mRNA vaccine, wherein the sexual life cycle of the malarial parasite is disrupted.

Disclosed herein is a method of interrupting malaria transmission, the method comprising administering to a subject a single disclosed mRNA vaccine or administering to a subject more than one disclosed mRNA vaccine in an amount effective to produce an antigen specific immune response in the subject, wherein the immune response is specific to the peptide or protein encoded by the mRNA vaccine, wherein the sexual life cycle of the malarial parasite is disrupted.

Disclosed herein is a method of interrupting malaria transmission, the method comprising administering to a subject in need thereof a therapeutically effective an mRNA vaccine, wherein the mRNA vaccine comprises at least one messenger ribonucleic acid (mRNA) encoding at least two antigenic peptides or proteins, wherein the at least two antigenic peptides or proteins are present in one or more life cycle stages of a malarial parasite, wherein the immune response is specific to the at least two antigenic peptides or proteins, wherein the sexual life cycle of the malarial parasite is disrupted.

Disclosed herein is a method of interrupting malaria transmission, the method comprising administering to a subject in need thereof a therapeutically effective an mRNA vaccine, wherein the mRNA vaccine comprises at least one messenger ribonucleic acid (mRNA) encoding at least two antigenic peptides or proteins, wherein the at least two antigenic peptides or proteins are present in one or more life cycle stages of a malarial parasite, wherein the disclosed mRNA molecule is encapsulated in a lipid nanoparticle, wherein the immune response is specific to the at least two antigenic peptides or proteins, wherein the sexual life cycle of the malarial parasite is disrupted.

Disclosed herein is a method of interrupting malaria transmission, the method comprising administering to a subject in need thereof a therapeutically effective an mRNA vaccine, wherein the mRNA vaccine comprises at least one messenger ribonucleic acid (mRNA) encoding at least two antigenic peptides or proteins, wherein the at least two antigenic peptides or proteins are present in one or more life cycle stages of a malarial parasite, wherein the mRNA vaccine comprises lipid nanoparticles (LNPs), and wherein the mRNA molecule is encapsulated in the LNPs, wherein the immune response is specific to the at least two antigenic peptides or proteins, wherein the sexual life cycle of the malarial parasite is disrupted.

Disclosed herein is a method of interrupting malaria transmission, the method comprising administering to a subject in need thereof a therapeutically effective an mRNA vaccine, wherein the mRNA vaccine comprises at least one messenger ribonucleic acid (mRNA) encoding at least two antigenic peptides or proteins, wherein the at least two antigenic peptides or proteins are present in one or more life cycle stages of a malarial parasite, wherein the mRNA molecule is encapsulated in a lipid nanoparticle, wherein the mRNA vaccine comprises lipid nanoparticles (LNPs), and wherein the mRNA molecule is encapsulated in the LNPs, wherein the immune response is specific to the at least two antigenic peptides or proteins, wherein the sexual life cycle of the malarial parasite is disrupted.

Disclosed herein is a method of interrupting malaria transmission, the method comprising administering to a subject in need thereof a therapeutically effective an mRNA vaccine, wherein the mRNA vaccine comprises a messenger RNA (mRNA) molecule, comprising: a first coding region encoding a first antigenic peptide or protein, and a second coding region encoding a second antigenic peptide or protein, wherein the first and second antigenic peptides or proteins are present in one or more life cycle stages of a malarial parasite, wherein the immune response is specific to the first antigenic peptide or protein and to the second first antigenic peptide or protein, wherein the sexual life cycle of the malarial parasite is disrupted.

Disclosed herein is a method of interrupting malaria transmission, the method comprising administering to a subject in need thereof a therapeutically effective a first mRNA vaccine, wherein the first mRNA vaccine comprises a messenger ribonucleic acid (mRNA) encoding a first antigenic peptide or protein, wherein the first antigenic peptide or protein is present in one or more life cycle stages of a malarial parasite, wherein the mRNA molecule is encapsulated in a lipid nanoparticle, and administering to the subject a therapeutically effective a second mRNA vaccine, wherein the second mRNA vaccine comprises a messenger ribonucleic acid (mRNA) encoding a second antigenic peptide or protein, wherein the second antigenic peptide or protein is present in one or more life cycle stages of a malarial parasite, wherein the mRNA molecule is encapsulated in a lipid nanoparticle, wherein the immune response is specific to the first antigenic peptide or protein and specific to the second antigenic peptide or protein, wherein the sexual life cycle of the malarial parasite is disrupted.

Disclosed herein is a method of interrupting malaria transmission, the method comprising administering to a subject in need thereof a therapeutically effective a first mRNA vaccine, wherein the first mRNA vaccine comprises a messenger ribonucleic acid (mRNA) encoding Plasmodium falciparum surface protein Pfs25, wherein the mRNA molecule is encapsulated in a lipid nanoparticle, and administering to the subject a therapeutically effective a second mRNA vaccine, wherein the second mRNA vaccine comprises a messenger ribonucleic acid (mRNA) encoding Plasmodium falciparum surface protein circumsporozoite protein (PfCSP), wherein the mRNA molecule is encapsulated in a lipid nanoparticle, wherein the immune response is specific to Pfs25 and PfCSP, wherein the sexual life cycle of the malarial parasite is disrupted.

Disclosed herein is a method of interrupting malaria transmission, the method comprising administering to a subject in need thereof a therapeutically effective a first mRNA vaccine, wherein the first mRNA vaccine comprises a messenger ribonucleic acid (mRNA) encoding Plasmodium falciparum surface protein Pfs25, wherein the encoded Pfs25 comprises the sequence set forth in SEQ ID NO:01 or SEQ ID NO:07, wherein the mRNA molecule is encapsulated in a lipid nanoparticle, and administering to the subject a therapeutically effective a second mRNA vaccine, wherein the second mRNA vaccine comprises a messenger ribonucleic acid (mRNA) encoding Plasmodium falciparum surface protein circumsporozoite protein (PfCSP), wherein the encoded PfCSP comprises the sequence set forth in SEQ ID NO:02 or SEQ ID NO:08, wherein the mRNA molecule is encapsulated in a lipid nanoparticle, wherein the immune response is specific to Pfs25 and PfCSP, wherein the sexual life cycle of the malarial parasite is disrupted.

Disclosed herein is a method of interrupting malaria transmission, the method comprising administering to a subject in need thereof a therapeutically effective a first mRNA vaccine, wherein the first mRNA vaccine comprises a messenger ribonucleic acid (mRNA) encoding Plasmodium falciparum surface protein Pfs25, wherein the encoded Pfs25 comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:01 or SEQ ID NO:07, wherein the mRNA molecule is encapsulated in a lipid nanoparticle, and administering to the subject a therapeutically effective a second mRNA vaccine, wherein the second mRNA vaccine comprises a messenger ribonucleic acid (mRNA) encoding Plasmodium falciparum surface protein circumsporozoite protein (PfCSP), wherein the encoded PfCSP comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:02 or SEQ ID NO:08, wherein the mRNA molecule is encapsulated in a lipid nanoparticle, wherein the immune response is specific to Pfs25 and PfCSP, wherein the sexual life cycle of the malarial parasite is disrupted.

Disclosed herein is a method of interrupting malaria transmission, the method comprising administering to a subject in need thereof a therapeutically effective a first mRNA vaccine, wherein the first mRNA vaccine comprises a messenger ribonucleic acid (mRNA) encoding Plasmodium falciparum surface protein Pfs25, wherein the encoded Pfs25 comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in GenBank Accession No. XP_001347587.1, EUT85305.1, AAD39544.1, UFQ05488.1, UFQ05499.1, ETW42495.1, QOQ86355.1, QRV62119.1, QRV62061.1, QOQ86414.1, QOQ86416.1, QRV62092.1, 6PHB_E, 6AZZ_A, AIV43628.1, AIV43629.1, AIV43624.1, AIV43630.1, AIV43626.1, AIV43634.1, ASU08938.1, AKJ86874.1, AIV43627.1, ASU08940.1, ASU08939.1, ALF99893.1, ASU09129.1, ASU09128.1, ASU09130.1, UIT08865.1, UIT08866.1, ALF99891.1, ALF99892.1, AIV43625.1, ALF99890.1, or AKJ86873.1, wherein the mRNA molecule is encapsulated in a lipid nanoparticle, and administering to the subject a therapeutically effective a second mRNA vaccine, wherein the second mRNA vaccine comprises a messenger ribonucleic acid (mRNA) encoding Plasmodium falciparum surface protein circumsporozoite protein (PfCSP), wherein the encoded PfCSP comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in GenBank Accession No. QOW08375.1, QOW08406.1, AAW78209.1, UFQ12080.1, QFZ93540.1, BAM84831.1, BAD73954.1, XP_001351122.1, BAM84814.1, AAW78190.1, AAW78206.1, UFQ12085.1, AAA29527.1, QFZ93502.1, UFQ12084.1, UFQ12082.1, QFZ93564.1, QFZ93554.1, AAW78200.1, AAF03135.1, AAW78193.1, ACO49498.1, AAN87576.1, ACO49324.1, BAM84859.1, AAN87615.1, ABF66070.1, BAM84771.1, ABF66086.1, QOW08441.1, QFZ93522.1, ABF66066.1, AAW78195.1, ABF66075.1, BAM84762.1, AAN87618.1, AAN87581.1, QOW08394.1, AAN87600.1, AAW78212.1, AW78211.1, AAN87622.1, ACO49332.1, ACO49330.1, KNG78490.1, AAN87620.1, BAD08405.1, QOW08419.1, ACO49375.1, AVC42000.1, P13814.1, ACO49327.1, QOW08472.1, QOW08469.1, AVC41974.1, AAA29547.1, BAM84971.1, ABF66089.1, ACO49331.1, AVC42006.1, ABF83989.1, BAM84962.1, ACO49325.1, ACO49339.1, AVC41971.1, AAA29543.1, AAA63422.1, ACO49409.1, BAM84856.1, ABF83997.1, AVC41992.1, ACO49410.1, or ABF83986.1, wherein the mRNA molecule is encapsulated in a lipid nanoparticle, wherein the immune response is specific to Pfs25 and PfCSP, wherein the sexual life cycle of the malarial parasite is disrupted.

Disclosed herein is a method of interrupting malaria transmission, the method comprising administering to a subject in need thereof a therapeutically effective a first mRNA vaccine, wherein the first mRNA vaccine comprises a messenger ribonucleic acid (mRNA) encoding Plasmodium falciparum surface protein Pfs25, wherein the encoded Pfs25 comprises the sequence set forth in GenBank Accession No. XP_001347587.1, EUT85305.1, AAD39544.1, UFQ05488.1, UFQ05499.1, ETW42495.1, QOQ86355.1, QRV62119.1, QRV62061.1, QOQ86414.1, QOQ86416.1, QRV62092.1, 6PHB_E, 6AZZ_A, AIV43628.1, AIV43629.1, AIV43624.1, AIV43630.1, AIV43626.1, AIV43634.1, ASU08938.1, AKJ86874.1, AIV43627.1, ASU08940.1, ASU08939.1, ALF99893.1, ASU09129.1, ASU09128.1, ASU09130.1, UIT08865.1, UIT08866.1, ALF99891.1, ALF99892.1, AIV43625.1, ALF99890.1, or AKJ86873.1, wherein the mRNA molecule is encapsulated in a lipid nanoparticle, and administering to the subject a therapeutically effective a second mRNA vaccine, wherein the second mRNA vaccine comprises a messenger ribonucleic acid (mRNA) encoding Plasmodium falciparum surface protein circumsporozoite protein (PfCSP), wherein the encoded PfCSP comprises the sequence set forth in GenBank Accession No. QOW08375.1, QOW08406.1, AAW78209.1, UFQ12080.1, QFZ93540.1, BAM84831.1, BAD73954.1, XP_001351122.1, BAM84814.1, AAW78190.1, AAW78206.1, UFQ12085.1, AAA29527.1, QFZ93502.1, UFQ12084.1, UFQ12082.1, QFZ93564.1, QFZ93554.1, AAW78200.1, AAF03135.1, AAW78193.1, ACO49498.1, AAN87576.1, ACO49324.1, BAM84859.1, AAN87615.1, ABF66070.1, BAM84771.1, ABF66086.1, QOW08441.1, QFZ93522.1, ABF66066.1, AAW78195.1, ABF66075.1, BAM84762.1, AAN87618.1, AAN87581.1, QOW08394.1, AAN87600.1, AAW78212.1, AW78211.1, AAN87622.1, ACO49332.1, ACO49330.1, KNG78490.1, AAN87620.1, BAD08405.1, QOW08419.1, ACO49375.1, AVC42000.1, P13814.1, ACO49327.1, QOW08472.1, QOW08469.1, AVC41974.1, AAA29547.1, BAM84971.1, ABF66089.1, ACO49331.1, AVC42006.1, ABF83989.1, BAM84962.1, ACO49325.1, ACO49339.1, AVC41971.1, AAA29543.1, AAA63422.1, ACO49409.1, BAM84856.1, ABF83997.1, AVC41992.1, ACO49410.1, or ABF83986.1, wherein the mRNA molecule is encapsulated in a lipid nanoparticle, wherein the immune response is specific to Pfs25 and PfCSP, wherein the sexual life cycle of the malarial parasite is disrupted.

Disclosed herein is a method of interrupting malaria transmission, the method comprising administering to a subject in need thereof a therapeutically effective a first mRNA vaccine, wherein the first mRNA vaccine comprises a lipid nanoparticle encapsulated messenger ribonucleic acid (mRNA) encoding Plasmodium falciparum surface protein Pfs25, and administering to the subject a therapeutically effective a second mRNA vaccine, wherein the second mRNA vaccine comprises a lipid nanoparticle encapsulated messenger ribonucleic acid (mRNA) encoding Plasmodium falciparum surface protein circumsporozoite protein (PfCSP), wherein the immune response is specific to Pfs25 and PfCSP, wherein the sexual life cycle of the malarial parasite is disrupted.

Disclosed herein is a method of interrupting malaria transmission, the method comprising administering to a subject in need thereof a therapeutically effective a first mRNA vaccine, wherein the first mRNA vaccine comprises a lipid nanoparticle encapsulated messenger ribonucleic acid (mRNA) encoding a first antigenic peptide or protein, wherein the first antigenic peptide or protein is present in one or more life cycle stages of a malarial parasite, and administering to the subject a therapeutically effective a second mRNA vaccine, wherein the second mRNA vaccine comprises a lipid nanoparticle encapsulated messenger ribonucleic acid (mRNA) encoding a second antigenic peptide or protein, wherein the second antigenic peptide or protein is present in one or more life cycle stages of a malarial parasite, wherein the immune response is specific to the first antigenic peptide or protein and specific to the second antigenic peptide or protein, wherein the sexual life cycle of the malarial parasite is disrupted.

Disclosed herein is a method of interrupting malaria transmission, the method comprising administering to a subject in need thereof a therapeutically effective a first mRNA vaccine, wherein the first mRNA vaccine comprises a lipid nanoparticle encapsulated messenger administering to the subject a therapeutically effective a second mRNA vaccine, wherein the second mRNA vaccine comprises a lipid nanoparticle encapsulated messenger ribonucleic acid (mRNA) encoding Plasmodium falciparum surface protein circumsporozoite protein (PfCSP), wherein the immune response is specific to Pfs25 and PfCSP, wherein the sexual life cycle of the malarial parasite is disrupted.

Disclosed herein is a method of interrupting malaria transmission, the method comprising administering to a subject in need thereof a therapeutically effective a first mRNA vaccine, wherein the first mRNA vaccine comprises a lipid nanoparticle encapsulated messenger ribonucleic acid (mRNA) encoding Plasmodium falciparum surface protein Pfs25, wherein the encoded Pfs25 comprises the sequence set forth in SEQ ID NO:01 or SEQ ID NO:07, and administering to the subject a therapeutically effective a second mRNA vaccine, wherein the second mRNA vaccine comprises a lipid nanoparticle encapsulated messenger ribonucleic acid (mRNA) encoding Plasmodium falciparum surface protein circumsporozoite protein (PfCSP), wherein the encoded PfCSP comprises the sequence set forth in SEQ ID NO:02 or SEQ ID NO:08, wherein the immune response is specific to Pfs25 and PfCSP, wherein the sexual life cycle of the malarial parasite is disrupted.

Disclosed herein is a method of interrupting malaria transmission, the method comprising administering to a subject in need thereof a therapeutically effective a first mRNA vaccine, wherein the first mRNA vaccine comprises a lipid nanoparticle encapsulated messenger ribonucleic acid (mRNA) encoding Plasmodium falciparum surface protein Pfs25, wherein the encoded Pfs25 comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:01 or SEQ ID NO:07, and administering to the subject a therapeutically effective a second mRNA vaccine, wherein the second mRNA vaccine comprises a lipid nanoparticle encapsulated messenger ribonucleic acid (mRNA) encoding Plasmodium falciparum surface protein circumsporozoite protein (PfCSP), wherein the encoded PfCSP comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:02 or SEQ ID NO:08, wherein the immune response is specific to Pfs25 and PfCSP, wherein the sexual life cycle of the malarial parasite is disrupted.

Disclosed herein is a method of interrupting malaria transmission, the method comprising administering to a subject in need thereof a therapeutically effective a first mRNA vaccine, wherein the first mRNA vaccine comprises a lipid nanoparticle encapsulated messenger ribonucleic acid (mRNA) encoding Plasmodium falciparum surface protein Pfs25, wherein the encoded Pfs25 comprises the sequence set forth in GenBank Accession No. XP_001347587.1, EUT85305.1, AAD39544.1, UFQ05488.1, UFQ05499.1, ETW42495.1, QOQ86355.1, QRV62119.1, QRV62061.1, QOQ86414.1, QOQ86416.1, QRV62092.1, 6PHB_E, 6AZZ_A, AIV43628.1, AIV43629.1, AIV43624.1, AIV43630.1, AIV43626.1, AIV43634.1, ASU08938.1, AKJ86874.1, AIV43627.1, ASU08940.1, ASU08939.1, ALF99893.1, ASU09129.1, ASU09128.1, ASU09130.1, UIT08865.1, UIT08866.1, ALF99891.1, ALF99892.1, AIV43625.1, ALF99890.1, or AKJ86873.1, and administering to the subject a therapeutically effective a second mRNA vaccine, wherein the second mRNA vaccine comprises a lipid nanoparticle encapsulated messenger ribonucleic acid (mRNA) encoding Plasmodium falciparum surface protein circumsporozoite protein (PfCSP), wherein the encoded PfCSP comprises the sequence set forth in GenBank Accession No. QOW08375.1, QOW08406.1, AAW78209.1, UFQ12080.1, QFZ93540.1, BAM84831.1, BAD73954.1, XP_001351122.1, BAM84814.1, AAW78190.1, AAW78206.1, UFQ12085.1, AAA29527.1, QFZ93502.1, UFQ12084.1, UFQ12082.1, QFZ93564.1, QFZ93554.1, AAW78200.1, AAF03135.1, AAW78193.1, ACO49498.1, AAN87576.1, ACO49324.1, BAM84859.1, AAN87615.1, ABF66070.1, BAM84771.1, ABF66086.1, QOW08441.1, QFZ93522.1, ABF66066.1, AAW78195.1, ABF66075.1, BAM84762.1, AAN87618.1, AAN87581.1, QOW08394.1, AAN87600.1, AAW78212.1, AW78211.1, AAN87622.1, ACO49332.1, ACO49330.1, KNG78490.1, AAN87620.1, BAD08405.1, QOW08419.1, ACO49375.1, AVC42000.1, P13814.1, ACO49327.1, QOW08472.1, QOW08469.1, AVC41974.1, AAA29547.1, BAM84971.1, ABF66089.1, ACO49331.1, AVC42006.1, ABF83989.1, BAM84962.1, ACO49325.1, ACO49339.1, AVC41971.1, AAA29543.1, AAA63422.1, ACO49409.1, BAM84856.1, ABF83997.1, AVC41992.1, ACO49410.1, or ABF83986.1, wherein the immune response is specific to Pfs25 and PfCSP, wherein the sexual life cycle of the malarial parasite is disrupted.

Disclosed herein is a method of interrupting malaria transmission, the method comprising administering to a subject in need thereof a therapeutically effective a first mRNA vaccine, wherein the first mRNA vaccine comprises a lipid nanoparticle encapsulated messenger ribonucleic acid (mRNA) encoding Plasmodium falciparum surface protein Pfs25, wherein the encoded Pfs25 comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in GenBank Accession No. XP_001347587.1, EUT85305.1, AAD39544.1, UFQ05488.1, UFQ05499.1, ETW42495.1, QOQ86355.1, QRV62119.1, QRV62061.1, QOQ86414.1, QOQ86416.1, QRV62092.1, 6PHB_E, 6AZZ_A, AIV43628.1, AIV43629.1, AIV43624.1, AIV43630.1, AIV43626.1, AIV43634.1, ASU08938.1, AKJ86874.1, AIV43627.1, ASU08940.1, ASU08939.1, ALF99893.1, ASU09129.1, ASU09128.1, ASU09130.1, UIT08865.1, UIT08866.1, ALF99891.1, ALF99892.1, AIV43625.1, ALF99890.1, or AKJ86873.1, and administering to the subject a therapeutically effective a second mRNA vaccine, wherein the second mRNA vaccine comprises a lipid nanoparticle encapsulated messenger ribonucleic acid (mRNA) encoding Plasmodium falciparum surface protein circumsporozoite protein (PfCSP), wherein the encoded PfCSP comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in GenBank Accession No. QOW08375.1, QOW08406.1, AAW78209.1, UFQ12080.1, QFZ93540.1, BAM84831.1, BAD73954.1, XP_001351122.1, BAM84814.1, AAW78190.1, AAW78206.1, UFQ12085.1, AAA29527.1, QFZ93502.1, UFQ12084.1, UFQ12082.1, QFZ93564.1, QFZ93554.1, AAW78200.1, AAF03135.1, AAW78193.1, ACO49498.1, AAN87576.1, ACO49324.1, BAM84859.1, AAN87615.1, ABF66070.1, BAM84771.1, ABF66086.1, QOW08441.1, QFZ93522.1, ABF66066.1, AAW78195.1, ABF66075.1, BAM84762.1, AAN87618.1, AAN87581.1, QOW08394.1, AAN87600.1, AAW78212.1, AW78211.1, AAN87622.1, ACO49332.1, ACO49330.1, KNG78490.1, AAN87620.1, BAD08405.1, QOW08419.1, ACO49375.1, AVC42000.1, P13814.1, ACO49327.1, QOW08472.1, QOW08469.1, AVC41974.1, AAA29547.1, BAM84971.1, ABF66089.1, ACO49331.1, AVC42006.1, ABF83989.1, BAM84962.1, ACO49325.1, ACO49339.1, AVC41971.1, AAA29543.1, AAA63422.1, ACO49409.1, BAM84856.1, ABF83997.1, AVC41992.1, ACO49410.1, or ABF83986.1, wherein the immune response is specific to Pfs25 and PfCSP, wherein the sexual life cycle of the malarial parasite is disrupted.

In an aspect, a disclosed method of interrupting malaria transmission, can further comprise administering to the subject artesunate, aremether-lumefantrine, clindamycin, doxycycline, atovaquone, chloroquine, mefloquine, quinine, primaquine, or any combination thereof.

In an aspect of a disclosed method of interrupting malaria transmission, administering a first mRNA vaccine occurs prior to administering a second mRNA vaccine. In an aspect of a disclosed method of interrupting malaria transmission, administering a first mRNA vaccine occurs concurrently with administering a second mRNA vaccine. In an aspect of a disclosed method of interrupting malaria transmission, administering a first mRNA vaccine occurs after administering a second mRNA vaccine. In an aspect of a disclosed method of interrupting malaria transmission, the schedule for administering the first mRNA vaccine and administering the second mRNA vaccine can be established prior to treatment. In an aspect of a disclosed method of interrupting malaria transmission, the schedule for administering the first mRNA vaccine and administering the second mRNA vaccine can be established prior to treatment and subsequently modified.

In an aspect, an effective amount of a disclosed mRNA vaccine can comprise a range from about 0.0005 mg/kg body weight to about 500 mg/kg body weight. In an aspect, a disclosed therapeutically effective dose can range from about 0.001 mg/kg body weight to about 400 mg/kg body weight, from about 0.001 mg/kg body weight to about 300 mg/kg body weight, from about 0.001 mg/kg body weight to about 200 mg/kg body weight, from about 0.001 mg/kg body weight to about 100 mg/kg body weight, from about 0.001 mg/kg body weight to about 90 mg/kg body weight, from about 0.001 kg/kg body weight to about 80 mg/kg body weight, from about 0.001 mg/kg body weight to about 70 mg/kg body weight, from about 0.001 mg/kg body weight to about 60 mg/kg body weight, from about 0.001 mg/kg body weight to about 50 mg/kg body weight, from about 0.001 mg/kg body weight to about 40 mg/kg body weight, from about 0.001 mg/kg body weight to about 30 mg/kg body weight, from about 0.001 mg/kg body weight to about 25 mg/kg body weight, from about 0.001 mg/kg body weight to about 20 mg/kg body weight, from about 0.001 mg/kg body weight to about 15 mg/kg body weight, from about 0.001 mg/kg body weight to about 10 mg/kg body weight, or from about 0.001 mg/kg body weight to about 5 mg/kg body weight.

In an aspect, an effective amount of a disclosed mRNA vaccine can be as low as 10 μg, administered for example as a single dose or as two 5 μg doses. In an aspect, an effective amount of a disclosed mRNA vaccine can comprise a range from about 10 μg to about 300 μg. For example, in an aspect, an effective amount can be a total dose of 10 μg, 20 μg, 25 μg, 30 μg, 35 μg, 40 μg, 45 μg, 50 μg, 55 μg, 60 μg, 65 μg, 70 30 μg, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg or 200 μg, 210 μg, 220 μg, 230 μg, 240 μg, 250 μg, 260 μg, 270 μg, 280 μg, 290 μg or 300 μg.

In an aspect, a disclosed mRNA vaccine can be administered and dosed in accordance with current medical practice, taking into account the clinical condition of the subject, the site and method of administration, the scheduling of administration, the subject's age, sex, body weight and other factors relevant to clinicians of ordinary skill in the art. In an aspect, an effective amount of a disclosed mRNA vaccine can be determined by such relevant considerations as are known to those of ordinary skill in experimental clinical research, pharmacological, clinical, and medical arts.

In an aspect, a subject can be suspected of having or can be diagnosed with having malaria. In an aspect, a disclosed subject can be symptomatic or asymptomatic. In an aspect, a subject can be a subject in need of treatment of malaria.

In an aspect, a disclosed method can further comprise repeating one or more administering steps. In an aspect, a disclosed administering step can be repeated one or more, two or more, three or more, four or more, or more than four times.

In an aspect, a disclosed method of interrupting malaria transmission can further comprise repeating one or more, two or more, three or more, four or more, or more than four times the administering of a disclosed first mRNA vaccine comprising a messenger ribonucleic acid (mRNA) encoding a disclosed first antigenic peptide or protein. For example, in an aspect, a disclosed method can further comprise repeating one or more, two or more, three or more, four or more, or more than four times the administering of a disclosed second mRNA vaccine comprising a messenger ribonucleic acid (mRNA) encoding a disclosed second antigenic peptide or protein. For example, in an aspect, a disclosed method can further comprise repeating one or more, two or more, three or more, four or more, or more than four times (i) the administering of a disclosed first mRNA vaccine comprising a messenger ribonucleic acid (mRNA) encoding a disclosed first antigenic peptide or protein, and (ii) the administering of a disclosed second mRNA vaccine comprising a messenger ribonucleic acid (mRNA) encoding a disclosed second antigenic peptide or protein.

In an aspect, a disclosed method can further comprise repeating one or more, two or more, three or more, four or more, or more than four times the administering of a disclosed mRNA vaccine comprising a messenger ribonucleic acid (mRNA) encoding Plasmodium falciparum surface protein Pfs25.

In an aspect, a disclosed method can further comprise repeating one or more, two or more, three or more, four or more, or more than four times the administering of a disclosed mRNA vaccine comprising a messenger ribonucleic acid (mRNA) encoding Plasmodium falciparum surface protein circumsporozoite protein (PfCSP).

For example, in an aspect, a disclosed method can further comprise repeating one or more, two or more, three or more, four or more, or more than four times (i) the administering of a disclosed mRNA vaccine comprising a messenger ribonucleic acid (mRNA) encoding Plasmodium falciparum surface protein Pfs25, and (ii) the administering of a disclosed mRNA vaccine comprises a messenger ribonucleic acid (mRNA) encoding Plasmodium falciparum surface protein circumsporozoite protein (PfCSP).

For example, in an aspect, a disclosed method can further comprise repeating one or more, two or more, three or more, four or more, or more than four times the administering of a disclosed mRNA vaccine comprising a messenger ribonucleic acid (mRNA) encoding Plasmodium falciparum surface protein Pfs25 wherein the encoded Pfs25 comprises the sequence set forth in SEQ ID NO:01 or SEQ ID NO:07. For example, in an aspect, a disclosed method can further comprise repeating one or more, two or more, three or more, four or more, or more than four times the administering of a disclosed mRNA vaccine comprising a messenger ribonucleic acid (mRNA) encoding Plasmodium falciparum surface protein circumsporozoite protein (PfCSP), wherein the encoded PfCSP comprises the sequence set forth in SEQ ID NO:02 or SEQ ID NO:08.

For example, in an aspect, a disclosed method can further comprise repeating one or more, two or more, three or more, four or more, or more than four times (i) the administering of a disclosed mRNA vaccine comprising a messenger ribonucleic acid (mRNA) encoding Plasmodium falciparum surface protein Pfs25, wherein the encoded Pfs25 comprises the sequence set forth in SEQ ID NO:01 or SEQ ID NO:07, and (ii) the administering of a disclosed mRNA vaccine comprises a messenger ribonucleic acid (mRNA) encoding Plasmodium falciparum surface protein circumsporozoite protein (PfCSP), wherein the encoded PfCSP comprises the sequence set forth in SEQ ID NO:02 or SEQ ID NO:08.

In an aspect, a disclosed method of interrupting malaria transmission can further comprise monitoring the subject for adverse effects. In an aspect, in the absence of adverse effects, the method can further comprise continuing to treat the subject and/or continuing to monitor the subject. In an aspect, in the presence of adverse effects, the method can further comprise modifying the treating step, modifying the administering step, or both. Methods of monitoring a subject's well-being can include both subjective and objective criteria (and are discussed supra). Such methods are known to the skilled person.

In an aspect, a disclosed method can further comprise administering to the subject a therapeutically effective amount of a therapeutic agent. Therapeutic agents are known.

In an aspect, a disclosed method can further comprise administering to the subject a therapeutically effective amount of one or more immune modulators. In an aspect, the one or more immune modulators comprise methotrexate, rituximab, intravenous gamma globulin, Tacrolimus, SVP-Rapamycin, bortezomib, or a combination thereof.

In an aspect, a disclosed method can further comprise validating and/or characterizing a disclosed mRNA vaccine. In an aspect, validating and/or charactering a disclosed mRNA vaccine can comprise determining the function and/or activity of the disclosed mRNA vaccine. In an aspect, validating and/or charactering a disclosed mRNA vaccine can comprise measuring and/or determining the efficacy of the disclosed mRNA vaccine. In an aspect, validating and/or charactering a disclosed mRNA vaccine can comprise measuring and/or determining the transmission blocking ability of the disclosed mRNA vaccine. In an aspect, validating and/or charactering a disclosed mRNA vaccine can comprise measuring and/or determining the transmission reducing activity of the disclosed mRNA vaccine. In an aspect, transmission reducing activity can be defined as the present reduction in mean oocysts between test IgG-fed mosquitoes and control IgG-fed mosquitoes. In an aspect, validating and/or charactering a disclosed mRNA vaccine can comprise measuring and/or determining the transmission blocking activity of the disclosed mRNA vaccine. In an aspect, transmission blocking activity can be defined as the present reduction in the proportion of infected mosquitoes between the test IgG-fed mosquitoes and control IgG-fed mosquitoes. In an aspect, validating and/or characterizing a disclosed mRNA vaccine and/or the function and/or activity of a disclosed mRNA vaccine can comprise using an animal model (such as, for example, mice, rats, hamsters, etc.). In an aspect, validating and/or characterizing a disclosed mRNA vaccine and/or the function and/or activity of a disclosed mRNA vaccine can comprise using a mosquito model.

In an aspect of a disclosed method of interrupting malaria transmission, administering a disclosed mRNA vaccine can comprise intravenous administration, intracerebral administration, intra-CSF administration, intracerebroventricular (ICV) administration, intraventricular administration, intra-cisterna magna (ICM) administration, intraparenchymal administration, intrathecal (lumbar, cisternal, or both) administration, intrahepatic administration, hepatic intra-arterial administration, hepatic portal vein (HPV) administration, or any combination thereof.

In an aspect of a disclosed method, a subject can be immunized with a single administration of a disclosed mRNA molecule, a disclosed mRNA vaccine, and/or a disclosed pharmaceutical formulation. In an aspect of a disclosed method, a subject can be immunized with two administrations of a disclosed mRNA molecule, a disclosed mRNA vaccine, and/or a disclosed pharmaceutical formulation. In an aspect of a disclosed method, a subject can be immunized with three administrations of a disclosed mRNA molecule, a disclosed mRNA vaccine, and/or a disclosed pharmaceutical formulation. In an aspect of a disclosed method, a subject can be immunized with four administrations of a disclosed mRNA molecule, a disclosed mRNA vaccine, and/or a disclosed pharmaceutical formulation. In an aspect of a disclosed method, a subject can be immunized with five administrations of a disclosed mRNA molecule, a disclosed mRNA vaccine, and/or a disclosed pharmaceutical formulation. In an aspect of a disclosed method, a subject can be immunized with more than 5 administrations of a disclosed mRNA molecule, a disclosed mRNA vaccine, and/or a disclosed pharmaceutical formulation.

In an aspect, a disclosed method of disrupting the sexual life cycle of the malarial parasite can employ multiple routes of administration to the subject. In an aspect, a disclosed method can employ a first route of administration that can be the same or different as a second and/or subsequent routes of administration. In an aspect, a disclosed mRNA molecule, a disclosed mRNA vaccine, a disclosed pharmaceutical formulation, or any combination thereof can be concurrently and/or serially administered to a subject via multiple routes of administration. For example, in an aspect, administering a disclosed mRNA molecule, a disclosed mRNA vaccine, and/or a disclosed pharmaceutical formulation can comprise intravenous administration and intra-cistern magna (ICM) administration. In an aspect, administering a disclosed mRNA molecule, a disclosed mRNA vaccine, and/or a disclosed pharmaceutical formulation can comprise IV administration and intrathecal (ITH) administration.

In an aspect, a disclosed method of interrupting malaria transmission can further comprise inducing an antigen specific immune response. In an aspect, a disclosed method of interrupting malaria transmission can further comprise establishing biodistribution, persistence, and integration analysis. In an aspect, a disclosed method of interrupting malaria transmission can further comprise performing assays to assess the distribution and duration of the immune response. In an aspect, a disclosed method of interrupting malaria transmission can further comprise disrupting and/or interrupting malaria transmission. In an aspect, a disclosed method of interrupting malaria transmission can comprise a partial disruption and/or interruption and/or a full disruption and/or interruption. In an aspect, a disclosed method of interrupting malaria transmission can further comprise reducing the pathological phenotype associated with malaria. In an aspect, a disclosed method of interrupting malaria transmission can further comprise diagnosing the subject with malaria. In an aspect, a disclosed method of interrupting malaria transmission can further comprise disrupting the sexual life cycle of the malarial parasite. In an aspect, a disclosed method of interrupting malaria transmission can further comprise causing the cessation and/or preventing and/or slowing the development of the malarial parasite. In an aspect, a disclosed method of interrupting malaria transmission can further comprise reducing the transmission of the malarial parasite. In an aspect, a disclosed method of interrupting malaria transmission can further comprise slowing malarial disease progression in a subject.

In an aspect, a disclosed method of interrupting malaria transmission can further comprise administering to the subject a therapeutically effective amount of an agent that can correct one or more aspects of a dysregulated metabolic or enzymatic pathway.

In an aspect, a disclosed method of interrupting malaria transmission can further comprise restoring one or more aspects of cellular homeostasis and/or cellular functionality and/or metabolic dysregulation. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of malaria; (vii) reversing, inhibiting, preventing, stabilizing, and/or slowing the rate of progression and/or transmission of malaria, or (viii) any combination thereof. In an aspect, restoring one or more aspects of cellular homeostasis can comprise improving, enhancing, restoring, and/or preserving one or more aspects of cellular structural and/or functional integrity. In an aspect of a disclosed method of interrupting malaria transmission, techniques to monitor, measure, and/or assess the restoring one or more aspects of cellular homeostasis and/or cellular functionality can comprise qualitative (or subjective) means as well as quantitative (or objective) means. These means are known to the skilled person. For example, representative regulated variables and sensors relating to systemic homeostasis are discussed supra.

In an aspect, a disclosed method of interrupting malaria transmission can further comprise repeating one or more steps of the method and/or modifying one or more steps of the method (such as, for example, an administering step).

In an aspect, a disclosed method of interrupting malaria transmission can further comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount of one or more of a disclosed mRNA molecule, a disclosed mRNA vaccine, a disclosed pharmaceutical formulation, or any combination thereof administered to a subject, or by changing the frequency of administration of one or more of a disclosed mRNA molecule, a disclosed mRNA vaccine, a disclosed pharmaceutical formulation, or any combination thereof to a subject, or by changing the duration of time one or more of a disclosed mRNA molecule, a disclosed mRNA vaccine, a disclosed pharmaceutical formulation, or any combination thereof are administered to a subject.

In an aspect, a disclosed method of interrupting malaria transmission can further comprise generating and/or validating one or more of the disclosed mRNA molecules, one or more of the disclosed mRNA vaccines, one or more of the disclosed pharmaceutical formulations, or any combination thereof.

In an aspect of a disclosed method of interrupting malaria transmission, a disclosed pharmaceutical formulation (such as, for example, a disclosed pharmaceutical formulation comprising a disclosed mRNA vaccine) can be substituted in lieu of a disclosed mRNA vaccine.

In an aspect of a disclosed method of interrupting malaria transmission a disclosed mRNA molecule can encode a homologue of Pfs25 and/or PfCSP. In an aspect, for example, a homologue can be Pvs25 (Plasmodium vivax) or can be PvCSP (Plasmodium vivax), both of which are discussed supra.

### H. Methods of Treating and/or Preventing Malaria

Disclosed herein is a method of treating and/or preventing malaria, the method comprising administering to a subject in need thereof a disclosed mRNA vaccine in an amount effective to treat and/or preventing malaria.

Disclosed herein is a method of treating and/or preventing malaria, the method comprising administering to a subject in need thereof an mRNA vaccine in an amount effective to produce an antigen specific immune response in the subject, wherein the immune response is specific to the peptide or protein encoded by the mRNA vaccine, wherein malaria is treated and/or prevented in the subject.

Disclosed herein is a method of treating and/or preventing malaria, the method comprising administering to a subject a single disclosed mRNA vaccine or administering to a subject more than one disclosed mRNA vaccine in an amount effective to produce an antigen specific immune response in the subject, wherein the immune response is specific to the peptide or protein encoded by the mRNA vaccine, wherein malaria is treated and/or prevented in the subject.

Disclosed herein is a method of treating and/or preventing malaria, the method comprising administering to a subject in need thereof a therapeutically effective an mRNA vaccine, wherein the mRNA vaccine comprises at least one messenger ribonucleic acid (mRNA) encoding at least two antigenic peptides or proteins, wherein the at least two antigenic peptides or proteins are present in one or more life cycle stages of a malarial parasite, wherein the immune response is specific to the at least two antigenic peptides or proteins, wherein malaria is treated and/or prevented in the subject.

Disclosed herein is a method of treating and/or preventing malaria, the method comprising administering to a subject in need thereof a therapeutically effective an mRNA vaccine, wherein the mRNA vaccine comprises at least one messenger ribonucleic acid (mRNA) encoding at least two antigenic peptides or proteins, wherein the at least two antigenic peptides or proteins are present in one or more life cycle stages of a malarial parasite, wherein the disclosed mRNA molecule is encapsulated in a lipid nanoparticle, wherein the immune response is specific to the at least two antigenic peptides or proteins, wherein malaria is treated and/or prevented in the subject.

Disclosed herein is a method of treating and/or preventing malaria, the method comprising administering to a subject in need thereof a therapeutically effective an mRNA vaccine, wherein the mRNA vaccine comprises at least one messenger ribonucleic acid (mRNA) encoding at least two antigenic peptides or proteins, wherein the at least two antigenic peptides or proteins are present in one or more life cycle stages of a malarial parasite, wherein the mRNA vaccine comprises lipid nanoparticles (LNPs), and wherein the mRNA molecule is encapsulated in the LNPs, wherein the immune response is specific to the at least two antigenic peptides or proteins, wherein malaria is treated and/or prevented in the subject.

Disclosed herein is a method of treating and/or preventing malaria, the method comprising administering to a subject in need thereof a therapeutically effective an mRNA vaccine, wherein the mRNA vaccine comprises at least one messenger ribonucleic acid (mRNA) encoding at least two antigenic peptides or proteins, wherein the at least two antigenic peptides or proteins are present in one or more life cycle stages of a malarial parasite, wherein the mRNA molecule is encapsulated in a lipid nanoparticle, wherein the mRNA vaccine comprises lipid nanoparticles (LNPs), and wherein the mRNA molecule is encapsulated in the LNPs, wherein the immune response is specific to the at least two antigenic peptides or proteins, wherein malaria is treated and/or prevented in the subject.

Disclosed herein is a method of treating and/or preventing malaria, the method comprising administering to a subject in need thereof a therapeutically effective an mRNA vaccine, wherein the mRNA vaccine comprises a messenger RNA (mRNA) molecule, comprising: a first coding region encoding a first antigenic peptide or protein, and a second coding region encoding a second antigenic peptide or protein, wherein the first and second antigenic peptides or proteins are present in one or more life cycle stages of a malarial parasite, wherein the immune response is specific to the first antigenic peptide or protein and to the second first antigenic peptide or protein, wherein malaria is treated and/or prevented in the subject.

Disclosed herein is a method of treating and/or preventing malaria, the method comprising administering to a subject in need thereof a therapeutically effective a first mRNA vaccine, wherein the first mRNA vaccine comprises a messenger ribonucleic acid (mRNA) encoding a first antigenic peptide or protein, wherein the first antigenic peptide or protein is present in one or more life cycle stages of a malarial parasite, wherein the mRNA molecule is encapsulated in a lipid nanoparticle, and administering to the subject a therapeutically effective a second mRNA vaccine, wherein the second mRNA vaccine comprises a messenger ribonucleic acid (mRNA) encoding a second antigenic peptide or protein, wherein the second antigenic peptide or protein is present in one or more life cycle stages of a malarial parasite, wherein the mRNA molecule is encapsulated in a lipid nanoparticle, wherein the immune response is specific to the first antigenic peptide or protein and specific to the second antigenic peptide or protein, wherein malaria is treated and/or prevented in the subject.

Disclosed herein is a method of treating and/or preventing malaria, the method comprising administering to a subject in need thereof a therapeutically effective a first mRNA vaccine, wherein the first mRNA vaccine comprises a messenger ribonucleic acid (mRNA) encoding Plasmodium falciparum surface protein Pfs25, wherein the mRNA molecule is encapsulated in a lipid nanoparticle, and administering to the subject a therapeutically effective a second mRNA vaccine, wherein the second mRNA vaccine comprises a messenger ribonucleic acid (mRNA) encoding Plasmodium falciparum surface protein circumsporozoite protein (PfCSP), wherein the mRNA molecule is encapsulated in a lipid nanoparticle, wherein the immune response is specific to Pfs25 and PfCSP, wherein malaria is treated and/or prevented in the subject.

Disclosed herein is a method of treating and/or preventing malaria, the method comprising administering to a subject in need thereof a therapeutically effective a first mRNA vaccine, wherein the first mRNA vaccine comprises a messenger ribonucleic acid (mRNA) encoding Plasmodium falciparum surface protein Pfs25, wherein the encoded Pfs25 comprises the sequence set forth in SEQ ID NO:01 or SEQ ID NO:07, wherein the mRNA molecule is encapsulated in a lipid nanoparticle, and administering to the subject a therapeutically effective a second mRNA vaccine, wherein the second mRNA vaccine comprises a messenger ribonucleic acid (mRNA) encoding Plasmodium falciparum surface protein circumsporozoite protein (PfCSP), wherein the encoded PfCSP comprises the sequence set forth in SEQ ID NO:02 or SEQ ID NO:08, wherein the mRNA molecule is encapsulated in a lipid nanoparticle, wherein the immune response is specific to Pfs25 and PfCSP, wherein malaria is treated and/or prevented in the subject.

Disclosed herein is a method of treating and/or preventing malaria, the method comprising administering to a subject in need thereof a therapeutically effective a first mRNA vaccine, wherein the first mRNA vaccine comprises a messenger ribonucleic acid (mRNA) encoding Plasmodium falciparum surface protein Pfs25, wherein the encoded Pfs25 comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:01 or SEQ ID NO:07, wherein the mRNA molecule is encapsulated in a lipid nanoparticle, and administering to the subject a therapeutically effective a second mRNA vaccine, wherein the second mRNA vaccine comprises a messenger ribonucleic acid (mRNA) encoding Plasmodium falciparum surface protein circumsporozoite protein (PfCSP), wherein the encoded PfCSP comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:02 or SEQ ID NO:08, wherein the mRNA molecule is encapsulated in a lipid nanoparticle, wherein the immune response is specific to Pfs25 and PfCSP, wherein malaria is treated and/or prevented in the subject.

Disclosed herein is a method of treating and/or preventing malaria, the method comprising administering to a subject in need thereof a therapeutically effective a first mRNA vaccine, wherein the first mRNA vaccine comprises a messenger ribonucleic acid (mRNA) encoding Plasmodium falciparum surface protein Pfs25, wherein the encoded Pfs25 comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in GenBank Accession No. XP_001347587.1, EUT85305.1, AAD39544.1, UFQ05488.1, UFQ05499.1, ETW42495.1, QOQ86355.1, QRV62119.1, QRV62061.1, QOQ86414.1, QOQ86416.1, QRV62092.1, 6PHB_E, 6AZZ_A, AIV43628.1, AIV43629.1, AIV43624.1, AIV43630.1, AIV43626.1, AIV43634.1, ASU08938.1, AKJ86874.1, AIV43627.1, ASU08940.1, ASU08939.1, ALF99893.1, ASU09129.1, ASU09128.1, ASU09130.1, UIT08865.1, UIT08866.1, ALF99891.1, ALF99892.1, AIV43625.1, ALF99890.1, or AKJ86873.1, wherein the mRNA molecule is encapsulated in a lipid nanoparticle, and administering to the subject a therapeutically effective a second mRNA vaccine, wherein the second mRNA vaccine comprises a messenger ribonucleic acid (mRNA) encoding Plasmodium falciparum surface protein circumsporozoite protein (PfCSP), wherein the encoded PfCSP comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in GenBank Accession No. QOW08375.1, QOW08406.1, AAW78209.1, UFQ12080.1, QFZ93540.1, BAM84831.1, BAD73954.1, XP_001351122.1, BAM84814.1, AAW78190.1, AAW78206.1, UFQ12085.1, AAA29527.1, QFZ93502.1, UFQ12084.1, UFQ12082.1, QFZ93564.1, QFZ93554.1, AAW78200.1, AAF03135.1, AAW78193.1, ACO49498.1, AAN87576.1, ACO49324.1, BAM84859.1, AAN87615.1, ABF66070.1, BAM84771.1, ABF66086.1, QOW08441.1, QFZ93522.1, ABF66066.1, AAW78195.1, ABF66075.1, BAM84762.1, AAN87618.1, AAN87581.1, QOW08394.1, AAN87600.1, AAW78212.1, AW78211.1, AAN87622.1, ACO49332.1, ACO49330.1, KNG78490.1, AAN87620.1, BAD08405.1, QOW08419.1, ACO49375.1, AVC42000.1, P13814.1, ACO49327.1, QOW08472.1, QOW08469.1, AVC41974.1, AAA29547.1, BAM84971.1, ABF66089.1, ACO49331.1, AVC42006.1, ABF83989.1, BAM84962.1, ACO49325.1, ACO49339.1, AVC41971.1, AAA29543.1, AAA63422.1, ACO49409.1, BAM84856.1, ABF83997.1, AVC41992.1, ACO49410.1, or ABF83986.1, wherein the mRNA molecule is encapsulated in a lipid nanoparticle, wherein the immune response is specific to Pfs25 and PfCSP, wherein malaria is treated and/or prevented in the subject.

Disclosed herein is a method of treating and/or preventing malaria, the method comprising administering to a subject in need thereof a therapeutically effective a first mRNA vaccine, wherein the first mRNA vaccine comprises a messenger ribonucleic acid (mRNA) encoding Plasmodium falciparum surface protein Pfs25, wherein the encoded Pfs25 comprises the sequence set forth in GenBank Accession No. XP_001347587.1, EUT85305.1, AAD39544.1, UFQ05488.1, UFQ05499.1, ETW42495.1, QOQ86355.1, QRV62119.1, QRV62061.1, QOQ86414.1, QOQ86416.1, QRV62092.1, 6PHB_E, 6AZZ_A, AIV43628.1, AIV43629.1, AIV43624.1, AIV43630.1, AIV43626.1, AIV43634.1, ASU08938.1, AKJ86874.1, AIV43627.1, ASU08940.1, ASU08939.1, ALF99893.1, ASU09129.1, ASU09128.1, ASU09130.1, UIT08865.1, UIT08866.1, ALF99891.1, ALF99892.1, AIV43625.1, ALF99890.1, or AKJ86873.1, wherein the mRNA molecule is encapsulated in a lipid nanoparticle, and administering to the subject a therapeutically effective a second mRNA vaccine, wherein the second mRNA vaccine comprises a messenger ribonucleic acid (mRNA) encoding Plasmodium falciparum surface protein circumsporozoite protein (PfCSP), wherein the encoded PfCSP comprises the sequence set forth in GenBank Accession No. QOW08375.1, QOW08406.1, AAW78209.1, UFQ12080.1, QFZ93540.1, BAM84831.1, BAD73954.1, XP_001351122.1, BAM84814.1, AAW78190.1, AAW78206.1, UFQ12085.1, AAA29527.1, QFZ93502.1, UFQ12084.1, UFQ12082.1, QFZ93564.1, QFZ93554.1, AAW78200.1, AAF03135.1, AAW78193.1, ACO49498.1, AAN87576.1, ACO49324.1, BAM84859.1, AAN87615.1, ABF66070.1, BAM84771.1, ABF66086.1, QOW08441.1, QFZ93522.1, ABF66066.1, AAW78195.1, ABF66075.1, BAM84762.1, AAN87618.1, AAN87581.1, QOW08394.1, AAN87600.1, AAW78212.1, AW78211.1, AAN87622.1, ACO49332.1, ACO49330.1, KNG78490.1, AAN87620.1, BAD08405.1, QOW08419.1, ACO49375.1, AVC42000.1, P13814.1, ACO49327.1, QOW08472.1, QOW08469.1, AVC41974.1, AAA29547.1, BAM84971.1, ABF66089.1, ACO49331.1, AVC42006.1, ABF83989.1, BAM84962.1, ACO49325.1, ACO49339.1, AVC41971.1, AAA29543.1, AAA63422.1, ACO49409.1, BAM84856.1, ABF83997.1, AVC41992.1, ACO49410.1, or ABF83986.1, wherein the mRNA molecule is encapsulated in a lipid nanoparticle, wherein the immune response is specific to Pfs25 and PfCSP, wherein malaria is treated and/or prevented in the subject.

Disclosed herein is a method of treating and/or preventing malaria, the method comprising administering to a subject in need thereof a therapeutically effective a first mRNA vaccine, wherein the first mRNA vaccine comprises a lipid nanoparticle encapsulated messenger ribonucleic acid (mRNA) encoding Plasmodium falciparum surface protein Pfs25, and administering to the subject a therapeutically effective a second mRNA vaccine, wherein the second mRNA vaccine comprises a lipid nanoparticle encapsulated messenger ribonucleic acid (mRNA) encoding Plasmodium falciparum surface protein circumsporozoite protein (PfCSP), wherein the immune response is specific to Pfs25 and PfCSP, wherein malaria is treated and/or prevented in the subject.

Disclosed herein is a method of treating and/or preventing malaria, the method comprising administering to a subject in need thereof a therapeutically effective a first mRNA vaccine, wherein the first mRNA vaccine comprises a lipid nanoparticle encapsulated messenger ribonucleic acid (mRNA) encoding a first antigenic peptide or protein, wherein the first antigenic peptide or protein is present in one or more life cycle stages of a malarial parasite, and administering to the subject a therapeutically effective a second mRNA vaccine, wherein the second mRNA vaccine comprises a lipid nanoparticle encapsulated messenger ribonucleic acid (mRNA) encoding a second antigenic peptide or protein, wherein the second antigenic peptide or protein is present in one or more life cycle stages of a malarial parasite, wherein the immune response is specific to the first antigenic peptide or protein and specific to the second antigenic peptide or protein, wherein malaria is treated and/or prevented in the subject.

Disclosed herein is a method of treating and/or preventing malaria, the method comprising administering to a subject in need thereof a therapeutically effective a first mRNA vaccine, wherein the first mRNA vaccine comprises a lipid nanoparticle encapsulated messenger ribonucleic acid (mRNA) encoding Plasmodium falciparum surface protein Pfs25, and administering to the subject a therapeutically effective a second mRNA vaccine, wherein the second mRNA vaccine comprises a lipid nanoparticle encapsulated messenger ribonucleic acid (mRNA) encoding Plasmodium falciparum surface protein circumsporozoite protein (PfCSP), wherein the immune response is specific to Pfs25 and PfCSP, wherein malaria is treated and/or prevented in the subject.

Disclosed herein is a method of treating and/or preventing malaria, the method comprising administering to a subject in need thereof a therapeutically effective a first mRNA vaccine, wherein the first mRNA vaccine comprises a lipid nanoparticle encapsulated messenger ribonucleic acid (mRNA) encoding Plasmodium falciparum surface protein Pfs25, wherein the encoded Pfs25 comprises the sequence set forth in SEQ ID NO:01 or SEQ ID NO:07, and administering to the subject a therapeutically effective a second mRNA vaccine, wherein the second mRNA vaccine comprises a lipid nanoparticle encapsulated messenger ribonucleic acid (mRNA) encoding Plasmodium falciparum surface protein circumsporozoite protein (PfCSP), wherein the encoded PfCSP comprises the sequence set forth in SEQ ID NO:02 or SEQ ID NO:08, wherein the immune response is specific to Pfs25 and PfCSP, wherein malaria is treated and/or prevented in the subject.

Disclosed herein is a method of treating and/or preventing malaria, the method comprising administering to a subject in need thereof a therapeutically effective a first mRNA vaccine, wherein the first mRNA vaccine comprises a lipid nanoparticle encapsulated messenger ribonucleic acid (mRNA) encoding Plasmodium falciparum surface protein Pfs25, wherein the encoded Pfs25 comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:01 or SEQ ID NO:07, and administering to the subject a therapeutically effective a second mRNA vaccine, wherein the second mRNA vaccine comprises a lipid nanoparticle encapsulated messenger ribonucleic acid (mRNA) encoding Plasmodium falciparum surface protein circumsporozoite protein (PfCSP), wherein the encoded PfCSP comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:02 or SEQ ID NO:08, wherein the immune response is specific to Pfs25 and PfCSP, wherein malaria is treated and/or prevented in the subject.

Disclosed herein is a method of treating and/or preventing malaria, the method comprising administering to a subject in need thereof a therapeutically effective a first mRNA vaccine, wherein the first mRNA vaccine comprises a lipid nanoparticle encapsulated messenger ribonucleic acid (mRNA) encoding Plasmodium falciparum surface protein Pfs25, wherein the encoded Pfs25 comprises the sequence set forth in GenBank Accession No. XP_001347587.1, EUT85305.1, AAD39544.1, UFQ05488.1, UFQ05499.1, ETW42495.1, QOQ86355.1, QRV62119.1, QRV62061.1, QOQ86414.1, QOQ86416.1, QRV62092.1, 6PHB_E, 6AZZ_A, AIV43628.1, AIV43629.1, AIV43624.1, AIV43630.1, AIV43626.1, AIV43634.1, ASU08938.1, AKJ86874.1, AIV43627.1, ASU08940.1, ASU08939.1, ALF99893.1, ASU09129.1, ASU09128.1, ASU09130.1, UIT08865.1, UIT08866.1, ALF99891.1, ALF99892.1, AIV43625.1, ALF99890.1, or AKJ86873.1, and administering to the subject a therapeutically effective a second mRNA vaccine, wherein the second mRNA vaccine comprises a lipid nanoparticle encapsulated messenger ribonucleic acid (mRNA) encoding Plasmodium falciparum surface protein circumsporozoite protein (PfCSP), wherein the encoded PfCSP comprises the sequence set forth in GenBank Accession No. QOW08375.1, QOW08406.1, AAW78209.1, UFQ12080.1, QFZ93540.1, BAM84831.1, BAD73954.1, XP_001351122.1, BAM84814.1, AAW78190.1, AAW78206.1, UFQ12085.1, AAA29527.1, QFZ93502.1, UFQ12084.1, UFQ12082.1, QFZ93564.1, QFZ93554.1, AAW78200.1, AAF03135.1, AAW78193.1, ACO49498.1, AAN87576.1, ACO49324.1, BAM84859.1, AAN87615.1, ABF66070.1, BAM84771.1, ABF66086.1, QOW08441.1, QFZ93522.1, ABF66066.1, AAW78195.1, ABF66075.1, BAM84762.1, AAN87618.1, AAN87581.1, QOW08394.1, AAN87600.1, AAW78212.1, AW78211.1, AAN87622.1, ACO49332.1, ACO49330.1, KNG78490.1, AAN87620.1, BAD08405.1, QOW08419.1, ACO49375.1, AVC42000.1, P13814.1, ACO49327.1, QOW08472.1, QOW08469.1, AVC41974.1, AAA29547.1, BAM84971.1, ABF66089.1, ACO49331.1, AVC42006.1, ABF83989.1, BAM84962.1, ACO49325.1, ACO49339.1, AVC41971.1, AAA29543.1, AAA63422.1, ACO49409.1, BAM84856.1, ABF83997.1, AVC41992.1, ACO49410.1, or ABF83986.1, wherein the immune response is specific to Pfs25 and PfCSP, wherein malaria is treated and/or prevented in the subject.

Disclosed herein is a method of treating and/or preventing malaria, the method comprising administering to a subject in need thereof a therapeutically effective a first mRNA vaccine, wherein the first mRNA vaccine comprises a lipid nanoparticle encapsulated messenger ribonucleic acid (mRNA) encoding Plasmodium falciparum surface protein Pfs25, wherein the encoded Pfs25 comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in GenBank Accession No. XP_001347587.1, EUT85305.1, AAD39544.1, UFQ05488.1, UFQ05499.1, ETW42495.1, QOQ86355.1, QRV62119.1, QRV62061.1, QOQ86414.1, QOQ86416.1, QRV62092.1, 6PHB_E, 6AZZ_A, AIV43628.1, AIV43629.1, AIV43624.1, AIV43630.1, AIV43626.1, AIV43634.1, ASU08938.1, AKJ86874.1, AIV43627.1, ASU08940.1, ASU08939.1, ALF99893.1, ASU09129.1, ASU09128.1, ASU09130.1, UIT08865.1, UIT08866.1, ALF99891.1, ALF99892.1, AIV43625.1, ALF99890.1, or AKJ86873.1, and administering to the subject a therapeutically effective a second mRNA vaccine, wherein the second mRNA vaccine comprises a lipid nanoparticle encapsulated messenger ribonucleic acid (mRNA) encoding Plasmodium falciparum surface protein circumsporozoite protein (PfCSP), wherein the encoded PfCSP comprises a sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in GenBank Accession No. QOW08375.1, QOW08406.1, AAW78209.1, UFQ12080.1, QFZ93540.1, BAM84831.1, BAD73954.1, XP_001351122.1, BAM84814.1, AAW78190.1, AAW78206.1, UFQ12085.1, AAA29527.1, QFZ93502.1, UFQ12084.1, UFQ12082.1, QFZ93564.1, QFZ93554.1, AAW78200.1, AAF03135.1, AAW78193.1, ACO49498.1, AAN87576.1, ACO49324.1, BAM84859.1, AAN87615.1, ABF66070.1, BAM84771.1, ABF66086.1, QOW08441.1, QFZ93522.1, ABF66066.1, AAW78195.1, ABF66075.1, BAM84762.1, AAN87618.1, AAN87581.1, QOW08394.1, AAN87600.1, AAW78212.1, AW78211.1, AAN87622.1, ACO49332.1, ACO49330.1, KNG78490.1, AAN87620.1, BAD08405.1, QOW08419.1, ACO49375.1, AVC42000.1, P13814.1, ACO49327.1, QOW08472.1, QOW08469.1, AVC41974.1, AAA29547.1, BAM84971.1, ABF66089.1, ACO49331.1, AVC42006.1, ABF83989.1, BAM84962.1, ACO49325.1, ACO49339.1, AVC41971.1, AAA29543.1, AAA63422.1, ACO49409.1, BAM84856.1, ABF83997.1, AVC41992.1, ACO49410.1, or ABF83986.1, wherein the immune response is specific to Pfs25 and PfCSP, wherein malaria is treated and/or prevented in the subject.

In an aspect, a disclosed method of treating and/or preventing malaria can further comprise administering to the subject artesunate, aremether-lumefantrine, clindamycin, doxycycline, atovaquone, chloroquine, mefloquine, quinine, primaquine, or any combination thereof.

In an aspect of a disclosed method of treating and/or preventing malaria, administering a first mRNA vaccine occurs prior to administering a second mRNA vaccine. In an aspect of a disclosed method of treating and/or preventing malaria, administering a first mRNA vaccine occurs concurrently with administering a second mRNA vaccine. In an aspect of a disclosed method of treating and/or preventing malaria, administering a first mRNA vaccine occurs after administering a second mRNA vaccine. In an aspect of a disclosed method of treating and/or preventing malaria, the schedule for administering the first mRNA vaccine and administering the second mRNA vaccine can be established prior to treatment. In an aspect of a disclosed method of treating and/or preventing malaria, the schedule for administering the first mRNA vaccine and administering the second mRNA vaccine can be established prior to treatment and subsequently modified.

In an aspect, an effective amount of a disclosed mRNA vaccine can comprise a range from about 0.0005 mg/kg body weight to about 500 mg/kg body weight. In an aspect, a disclosed therapeutically effective dose can range from about 0.001 mg/kg body weight to about 400 mg/kg body weight, from about 0.001 mg/kg body weight to about 300 mg/kg body weight, from about 0.001 mg/kg body weight to about 200 mg/kg body weight, from about 0.001 mg/kg body weight to about 100 mg/kg body weight, from about 0.001 mg/kg body weight to about 90 mg/kg body weight, from about 0.001 kg/kg body weight to about 80 mg/kg body weight, from about 0.001 mg/kg body weight to about 70 mg/kg body weight, from about 0.001 mg/kg body weight to about 60 mg/kg body weight, from about 0.001 mg/kg body weight to about 50 mg/kg body weight, from about 0.001 mg/kg body weight to about 40 mg/kg body weight, from about 0.001 mg/kg body weight to about 30 mg/kg body weight, from about 0.001 mg/kg body weight to about 25 mg/kg body weight, from about 0.001 mg/kg body weight to about 20 mg/kg body weight, from about 0.001 mg/kg body weight to about 15 mg/kg body weight, from about 0.001 mg/kg body weight to about 10 mg/kg body weight, or from about 0.001 mg/kg body weight to about 5 mg/kg body weight.

In an aspect, an effective amount of a disclosed mRNA vaccine can be as low as 10 μg, administered for example as a single dose or as two 5 μg doses. In an aspect, an effective amount of a disclosed mRNA vaccine can comprise a range from about 10 μg to about 300 μg. For example, in an aspect, an effective amount can be a total dose of 10 μg, 20 μg, 25 μg, 30 μg, 35 μg, 40 μg, 45 μg, 50 μg, 55 μg, 60 μg, 65 μg, 70 30 μg, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg or 200 μg, 210 μg, 220 μg, 230 μg, 240 μg, 250 μg, 260 μg, 270 μg, 280 μg, 290 μg or 300 μg.

In an aspect, a disclosed mRNA vaccine can be administered and dosed in accordance with current medical practice, taking into account the clinical condition of the subject, the site and method of administration, the scheduling of administration, the subject's age, sex, body weight and other factors relevant to clinicians of ordinary skill in the art. In an aspect, an effective amount of a disclosed mRNA vaccine can be determined by such relevant considerations as are known to those of ordinary skill in experimental clinical research, pharmacological, clinical, and medical arts.

In an aspect, a subject can be suspected of having or can be diagnosed with having malaria. In an aspect, a disclosed subject can be symptomatic or asymptomatic. In an aspect, a subject can be a subject in need of treatment of malaria.

In an aspect, a disclosed method can further comprise repeating one or more administering steps. In an aspect, a disclosed administering step can be repeated one or more, two or more, three or more, four or more, or more than four times.

In an aspect, a disclosed method of treating and/or preventing malaria can further comprise repeating one or more, two or more, three or more, four or more, or more than four times the administering of a disclosed first mRNA vaccine comprising a messenger ribonucleic acid (mRNA) encoding a disclosed first antigenic peptide or protein. For example, in an aspect, a disclosed method can further comprise repeating one or more, two or more, three or more, four or more, or more than four times the administering of a disclosed second mRNA vaccine comprising a messenger ribonucleic acid (mRNA) encoding a disclosed second antigenic peptide or protein. For example, in an aspect, a disclosed method can further comprise repeating one or more, two or more, three or more, four or more, or more than four times (i) the administering of a disclosed first mRNA vaccine comprising a messenger ribonucleic acid (mRNA) encoding a disclosed first antigenic peptide or protein, and (ii) the administering of a disclosed second mRNA vaccine comprising a messenger ribonucleic acid (mRNA) encoding a disclosed second antigenic peptide or protein.

In an aspect, a disclosed method can further comprise repeating one or more, two or more, three or more, four or more, or more than four times the administering of a disclosed mRNA vaccine comprising a messenger ribonucleic acid (mRNA) encoding Plasmodium falciparum surface protein Pfs25.

In an aspect, a disclosed method can further comprise repeating one or more, two or more, three or more, four or more, or more than four times the administering of a disclosed mRNA vaccine comprising a messenger ribonucleic acid (mRNA) encoding Plasmodium falciparum surface protein circumsporozoite protein (PfCSP).

For example, in an aspect, a disclosed method can further comprise repeating one or more, two or more, three or more, four or more, or more than four times (i) the administering of a disclosed mRNA vaccine comprising a messenger ribonucleic acid (mRNA) encoding Plasmodium falciparum surface protein Pfs25, and (ii) the administering of a disclosed mRNA vaccine comprises a messenger ribonucleic acid (mRNA) encoding Plasmodium falciparum surface protein circumsporozoite protein (PfCSP).

For example, in an aspect, a disclosed method can further comprise repeating one or more, two or more, three or more, four or more, or more than four times the administering of a disclosed mRNA vaccine comprising a messenger ribonucleic acid (mRNA) encoding Plasmodium falciparum surface protein Pfs25 wherein the encoded Pfs25 comprises the sequence set forth in SEQ ID NO:01 or SEQ ID NO:07. For example, in an aspect, a disclosed method can further comprise repeating one or more, two or more, three or more, four or more, or more than four times the administering of a disclosed mRNA vaccine comprising a messenger ribonucleic acid (mRNA) encoding Plasmodium falciparum surface protein circumsporozoite protein (PfCSP), wherein the encoded PfCSP comprises the sequence set forth in SEQ ID NO:02 or SEQ ID NO:08.

For example, in an aspect, a disclosed method can further comprise repeating one or more, two or more, three or more, four or more, or more than four times (i) the administering of a disclosed mRNA vaccine comprising a messenger ribonucleic acid (mRNA) encoding Plasmodium falciparum surface protein Pfs25, wherein the encoded Pfs25 comprises the sequence set forth in SEQ ID NO:01 or SEQ ID NO:07, and (ii) the administering of a disclosed mRNA vaccine comprises a messenger ribonucleic acid (mRNA) encoding Plasmodium falciparum surface protein circumsporozoite protein (PfCSP), wherein the encoded PfCSP comprises the sequence set forth in SEQ ID NO:02 or SEQ ID NO:08.

In an aspect, a disclosed method of treating and/or preventing malaria can further comprise monitoring the subject for adverse effects. In an aspect, in the absence of adverse effects, the method can further comprise continuing to treat the subject and/or continuing to monitor the subject. In an aspect, in the presence of adverse effects, the method can further comprise modifying the treating step, modifying the administering step, or both. Methods of monitoring a subject's well-being can include both subjective and objective criteria (and are discussed supra). Such methods are known to the skilled person.

In an aspect, a disclosed method can further comprise administering to the subject a therapeutically effective amount of a therapeutic agent. Therapeutic agents are known.

In an aspect, a disclosed method can further comprise administering to the subject a therapeutically effective amount of one or more immune modulators. In an aspect, the one or more immune modulators comprise methotrexate, rituximab, intravenous gamma globulin, Tacrolimus, SVP-Rapamycin, bortezomib, or a combination thereof.

In an aspect, a disclosed method can further comprise validating and/or characterizing a disclosed mRNA vaccine. In an aspect, validating and/or charactering a disclosed mRNA vaccine can comprise determining the function and/or activity of the disclosed mRNA vaccine. In an aspect, validating and/or charactering a disclosed mRNA vaccine can comprise measuring and/or determining the efficacy of the disclosed mRNA vaccine. In an aspect, validating and/or charactering a disclosed mRNA vaccine can comprise measuring and/or determining the transmission blocking ability of the disclosed mRNA vaccine. In an aspect, validating and/or charactering a disclosed mRNA vaccine can comprise measuring and/or determining the transmission reducing activity of the disclosed mRNA vaccine. In an aspect, transmission reducing activity can be defined as the present reduction in mean oocysts between test IgG-fed mosquitoes and control IgG-fed mosquitoes. In an aspect, validating and/or charactering a disclosed mRNA vaccine can comprise measuring and/or determining the transmission blocking activity of the disclosed mRNA vaccine. In an aspect, transmission blocking activity can be defined as the present reduction in the proportion of infected mosquitoes between the test IgG-fed mosquitoes and control IgG-fed mosquitoes. In an aspect, validating and/or characterizing a disclosed mRNA vaccine and/or the function and/or activity of a disclosed mRNA vaccine can comprise using an animal model (such as, for example, mice, rats, hamsters, etc.). In an aspect, validating and/or characterizing a disclosed mRNA vaccine and/or the function and/or activity of a disclosed mRNA vaccine can comprise using a mosquito model.

In an aspect of a disclosed method of treating and/or preventing malaria, administering a disclosed mRNA vaccine can comprise intravenous administration, intracerebral administration, intra-CSF administration, intracerebroventricular (ICV) administration, intraventricular administration, intra-cisterna magna (ICM) administration, intraparenchymal administration, intrathecal (lumbar, cisternal, or both) administration, intrahepatic administration, hepatic intra-arterial administration, hepatic portal vein (HPV) administration, or any combination thereof.

In an aspect of a disclosed method, a subject can be immunized with a single administration of a disclosed mRNA molecule, a disclosed mRNA vaccine, and/or a disclosed pharmaceutical formulation. In an aspect of a disclosed method, a subject can be immunized with two administrations of a disclosed mRNA molecule, a disclosed mRNA vaccine, and/or a disclosed pharmaceutical formulation. In an aspect of a disclosed method, a subject can be immunized with three administrations of a disclosed mRNA molecule, a disclosed mRNA vaccine, and/or a disclosed pharmaceutical formulation. In an aspect of a disclosed method, a subject can be immunized with four administrations of a disclosed mRNA molecule, a disclosed mRNA vaccine, and/or a disclosed pharmaceutical formulation. In an aspect of a disclosed method, a subject can be immunized with five administrations of a disclosed mRNA molecule, a disclosed mRNA vaccine, and/or a disclosed pharmaceutical formulation. In an aspect of a disclosed method, a subject can be immunized with more than 5 administrations of a disclosed mRNA molecule, a disclosed mRNA vaccine, and/or a disclosed pharmaceutical formulation.

In an aspect, a disclosed method of treating and/or preventing malaria can employ multiple routes of administration to the subject. In an aspect, a disclosed method can employ a first route of administration that can be the same or different as a second and/or subsequent routes of administration. In an aspect, a disclosed mRNA molecule, a disclosed mRNA vaccine, a disclosed pharmaceutical formulation, or any combination thereof can be concurrently and/or serially administered to a subject via multiple routes of administration. For example, in an aspect, administering a disclosed mRNA molecule, a disclosed mRNA vaccine, and/or a disclosed pharmaceutical formulation can comprise intravenous administration and intra-cistern magna (ICM) administration. In an aspect, administering a disclosed mRNA molecule, a disclosed mRNA vaccine, and/or a disclosed pharmaceutical formulation can comprise IV administration and intrathecal (ITH) administration.

In an aspect, a disclosed method of treating and/or preventing malaria can further comprise inducing an antigen specific immune response. In an aspect a disclosed method of treating and/or preventing malaria can further comprise establishing biodistribution, persistence, and integration analysis. In an aspect, a disclosed method of treating and/or preventing malaria can further comprise performing assays to assess the distribution and duration of the immune response. In an aspect, a disclosed method of treating and/or preventing malaria can further comprise disrupting and/or interrupting malaria transmission. In an aspect, a disclosed method of treating and/or preventing malaria can comprise a partial disruption and/or interruption and/or a full disruption and/or interruption. In an aspect, a disclosed method of treating and/or preventing malaria can further comprise reducing the pathological phenotype associated with malaria. In an aspect, a disclosed method of treating and/or preventing malaria can further comprise diagnosing the subject with malaria. In an aspect, a disclosed method of treating and/or preventing malaria can further comprise disrupting the sexual life cycle of the malarial parasite. In an aspect, a disclosed method of treating and/or preventing malaria can further comprise causing the cessation and/or preventing and/or slowing the development of the malarial parasite. In an aspect, a disclosed method of treating and/or preventing malaria can further comprise reducing the transmission of the malarial parasite. In an aspect, a disclosed method of treating and/or preventing malaria can further comprise slowing malarial disease progression in a subject.

In an aspect, a disclosed method of treating and/or preventing malaria can further comprise administering to the subject a therapeutically effective amount of an agent that can correct one or more aspects of a dysregulated metabolic or enzymatic pathway.

In an aspect, a disclosed method of treating and/or preventing malaria can further comprise restoring one or more aspects of cellular homeostasis and/or cellular functionality and/or metabolic dysregulation. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of malaria; (vii) reversing, inhibiting, preventing, stabilizing, and/or slowing the rate of progression and/or transmission of malaria, or (viii) any combination thereof. In an aspect, restoring one or more aspects of cellular homeostasis can comprise improving, enhancing, restoring, and/or preserving one or more aspects of cellular structural and/or functional integrity. In an aspect of a disclosed method of treating and/or preventing malaria, techniques to monitor, measure, and/or assess the restoring one or more aspects of cellular homeostasis and/or cellular functionality can comprise qualitative (or subjective) means as well as quantitative (or objective) means. These means are known to the skilled person. For example, representative regulated variables and sensors relating to systemic homeostasis are discussed supra.

In an aspect, a disclosed method of treating and/or preventing malaria can further comprise repeating one or more steps of the method and/or modifying one or more steps of the method (such as, for example, an administering step).

In an aspect, a disclosed method of treating and/or preventing malaria can further comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount of one or more of a disclosed mRNA molecule, a disclosed mRNA vaccine, a disclosed pharmaceutical formulation, or any combination thereof administered to a subject, or by changing the frequency of administration of one or more of a disclosed mRNA molecule, a disclosed mRNA vaccine, a disclosed pharmaceutical formulation, or any combination thereof to a subject, or by changing the duration of time one or more of a disclosed mRNA molecule, a disclosed mRNA vaccine, a disclosed pharmaceutical formulation, or any combination thereof are administered to a subject.

In an aspect, a disclosed method of treating and/or preventing malaria can further comprise generating and/or validating one or more of the disclosed mRNA molecules, one or more of the disclosed mRNA vaccines, one or more of the disclosed pharmaceutical formulations, or any combination thereof.

In an aspect of a disclosed method of treating and/or preventing malaria, a disclosed pharmaceutical formulation (such as, for example, a disclosed pharmaceutical formulation comprising a disclosed mRNA vaccine) can be substituted in lieu of a disclosed mRNA vaccine.

In an aspect of a disclosed method of treating and/or preventing malaria, a disclosed mRNA molecule can encode a homologue of Pfs25 and/or PfCSP. In an aspect, for example, a homologue can be Pvs25 (Plasmodium vivax) or can be PvCSP (Plasmodium vivax), both of which are discussed supra.

### I. Kits

Disclosed herein is a kit comprising one or more disclosed mRNA molecules, disclosed mRNA vaccines, disclosed pharmaceutical formulations, disclosed host cells, disclosed plasmids, or any combination thereof with or without additional therapeutic agents to induce an antigen specific immune response. Disclosed herein is a kit comprising one or more disclosed mRNA molecules, disclosed mRNA vaccines, disclosed pharmaceutical formulations, disclosed host cells, disclosed plasmids, or any combination thereof with or without additional therapeutic agents to interrupt malaria transmission. Disclosed herein is a kit comprising one or more disclosed mRNA molecules, disclosed mRNA vaccines, disclosed pharmaceutical formulations, disclosed host cells, disclosed plasmids, or any combination thereof with or without additional therapeutic agents to treat and/or prevent malaria. Disclosed herein is a kit comprising one or more disclosed mRNA molecules, disclosed mRNA vaccines, disclosed pharmaceutical formulations, disclosed host cells, disclosed plasmids, or any combination thereof with or without additional therapeutic agents establish biodistribution, persistence, and integration analysis. Disclosed herein is a kit comprising one or more disclosed mRNA molecules, disclosed mRNA vaccines, disclosed pharmaceutical formulations, disclosed host cells, disclosed plasmids, or any combination thereof with or without additional therapeutic agents to disrupt the sexual life cycle of the malarial parasite. Disclosed herein is a kit comprising one or more disclosed mRNA molecules, disclosed mRNA vaccines, disclosed pharmaceutical formulations, disclosed host cells, disclosed plasmids, or any combination thereof with or without additional therapeutic agents to reduce the transmission of the malarial parasite. Disclosed herein is a kit comprising one or more disclosed mRNA molecules, disclosed mRNA vaccines, disclosed pharmaceutical formulations, disclosed host cells, disclosed plasmids, or any combination thereof with or without additional therapeutic agents to slow malarial disease progression in a subject.

In an aspect, a disclosed kit can comprise at least two components constituting the kit. Together, the components constitute a functional unit for a given purpose (such as, for example, treating a subject diagnosed with or suspected of having malaria). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise one or more containers holding a disclosed mRNA molecule, a disclosed mRNA vaccine, a disclosed pharmaceutical formulation, a disclosed therapeutic agent, a disclosed reagent, or a combination thereof, and a label or package insert with instructions for use. In an aspect, suitable containers include, for example, bottles, vials, syringes, blister pack, etc. The containers can be formed from a variety of materials such as glass or plastic. The container can hold, for example, a disclosed pharmaceutical formulation and/or a disclosed therapeutic agent and can have a sterile access port (for example the container may be an intravenous solution bag or a vial having a stopper pierceable by a hypodermic injection needle). The label or package insert can indicate that a disclosed pharmaceutical formulation and/or a disclosed therapeutic agent can be used for treating, preventing, inhibiting, and/or ameliorating malaria or complications and/or symptoms associated with malaria. In an aspect, a disclosed kit can comprise additional components necessary for administration such as, for example, other buffers, diluents, filters, needles, and syringes. In an aspect, a disclosed kit can comprise those components (e.g., primers) necessary to measure one or more times the development and/or transmission of malaria. In an aspect, a disclosed kit can comprise a mRNA molecule encoding a homologue of Pfs25 and/or PfCSP. In an aspect, for example, a homologue can be Pvs25 (Plasmodium vivax) or can be PvCSP (Plasmodium vivax), both of which are discussed supra.

TABLE 1
Sequence Information

| SEQ ID NO: | Name of Sequence | Type | Source |
|---|---|---|---|
| 1 | ookinete surface protein P25 (3D7) | AA | Plasmodium falciparum |
| 2 | circumsporozoite (CS) protein [3D7] | AA | Plasmodium falciparum |
| 3 | N-Terminal Signal (for Pfs25) | AA | synthetic/artificial |
| 4 | N-Terminal Signal (for PfCSP) | AA | synthetic/artificial |
| 5 | C-Terminal GPI Anchor (for Pfs25) | AA | synthetic/artificial |
| 6 | C-Terminal GPI Anchor (for PfCSP) | AA | synthetic/artificial |
| 7 | Pfs25 with N-Terminal Signal and C- | AA | synthetic/artificial |
| | Terminal GPI Anchor | | |
| 8 | PfCSP with N-Terminal Signal and C- | AA | synthetic/artificial |
| | Terminal GPI Anchor | | |
| 9 | ookinete surface protein P25 (3D7) | NT | Plasmodium falciparum |
| 10 | circumsporozoite (CS) protein [3D7] | NT | Plasmodium falciparum |
| 11 | VR1020 plasmid comprising PfCSP | NT | synthetic/artificial |
| 12 | VR1020 plasmid comprising Pfs25 | NT | synthetic/artificial |
| 13 | Pfs25 with Histidine Tag & Linker | AA | synthetic/artificial |
| 14 | Pfs25 Peptide | AA | Plasmodium falciparum |
| 15 | Pfs25 Peptide | AA | Plasmodium falciparum |
| 16 | Pfs25 Peptide | AA | Plasmodium falciparum |
| 17 | Pfs25 Peptide | AA | Plasmodium falciparum |
| 18 | Pfs25 Peptide | AA | Plasmodium falciparum |
| 19 | Pfs25 Peptide | AA | Plasmodium falciparum |
| 20 | Pfs25 Peptide | AA | Plasmodium falciparum |
| 21 | Pfs25 Peptide | AA | Plasmodium falciparum |
| 22 | Pfs25 Peptide | AA | Plasmodium falciparum |
| 23 | Pfs25 Peptide | AA | Plasmodium falciparum |
| 24 | Pfs25 Peptide | AA | Plasmodium falciparum |
| 25 | Pfs25 Peptide | AA | Plasmodium falciparum |
| 26 | Pfs25 Peptide | AA | Plasmodium falciparum |
| 27 | Pfs25 Peptide | AA | Plasmodium falciparum |
| 28 | Pfs25 Peptide | AA | Plasmodium falciparum |
| 29 | Pfs25 Peptide | AA | Plasmodium falciparum |
| 30 | Pfs25 Peptide | AA | Plasmodium falciparum |
| 31 | Spacer/Linker | AA | synthetic/artificial |

### EXAMPLES

Malaria is caused by Plasmodium parasites transmitted by female anopheline mosquitoes. As of 2020, malaria was prevalent in greater than 90 countries accounting for 241 million cases with an estimated 627,000 deaths (WHO. Global technical strategy for malaria 2016-2030 (2021); WHO. World malaria report 2021). Over the past few decades, progress has been made toward reducing malaria incidence through mosquito control interventions and increased access to antimalarial drugs. Unfortunately, drug resistance towards frontline antimalarial drugs continues to increase, and overall progress in incidence reduction has started to stagnate (WHO. World Malaria Report 2021; Balikagala B, et al. (2021) New Engl J Med. 385:1163-1171). Therefore, interventions, such as vaccines, are needed to achieve further progress toward malaria elimination.

There has been progress in vaccine development, where the RTS,S/AS01 vaccine became the first and only approved vaccine to combat the disease (WHO. World malaria report 2021). However, the RTS,S/AS01 vaccine is only partially effective in protecting against clinical malaria with the efficacy waning over multiple years (RTS,S Clinical Trials Partnership. (2015) Lancet. 386:31-45; Laurens M B. (2020) Hum Vaccin Immunother. 16:480-489).

Due to the complex parasite life cycle, there are three distinct types of vaccines in development: pre-erythrocytic, blood-stage, and transmission-blocking vaccines. Pre-erythrocytic vaccines target sporozoite and liver-stage parasites with the aim of eliciting immune responses to prevent infection. Blood-stage vaccines target the disease-causing blood-stage parasites to elicit an immune response to limit blood-stage parasite burden, thereby reducing disease severity. Transmission-blocking vaccines target the sexual stage parasites in the female mosquitoes, leading to the disruption of the sexual life cycle and cessation of parasite development, and reduction of transmission. It is widely accepted that a vaccine comprised of multiple antigen combinations and targeting multiple stages will likely produce a highly effective vaccine, to stop malaria transmission (Yusuf Y, et al. (2019) Front Immunol. 10:2412; Boes A, et al. (2015) PLOS One. 10:e0131456; Spiegel H, et al. (2015) Biotechnol J. 10(10):1651-1659; Brod F, et al. (2018) Front Immunol. 9:2780).

One of the primary targets for pre-erythrocytic vaccine development is the Plasmodium falciparum circumsporozoite protein (PfCSP). PfCSP is a protein containing three regions; a N terminal domain, and immunodominant repeat region and a C terminal domain which contains multiple T cell epitopes (Plassmeyer M L, et al. (2009) J Biol Chem. 284:26951-26963). PfCSP is expressed on the infectious sporozoite stage that contributes to parasite motility and hepatocyte invasion (Wardemann H, et al. (2018) Curr Opin Immunol. 53:119-123). Numerous vaccine platforms, such as virus-like particles (VLPs), nanoparticles, live vectors, and DNA plasmids, have been evaluated for PfCSP vaccine development with mixed success (Yusuf Y, et al. (2019) Front Immunol. 10:2412; Molina-Franky J, et al. (2020) Malar J. 19(1):56; Ferraro B, et al. (2013) Infect Immun. 81(10):3709-3720; McCoy M E, et al. (2013) Malaria J. 12:136).

Pfs25 is one of the few leading targets for transmission-blocking vaccines, alongside Pfs48/45 and Pfs230 (Duffy P E. (2021) Expert Rev Vaccines. 20(2):185-198). Pfs25 is a cysteine rich protein consisting of four EGF-like domains.

Pfs25 is expressed on the surface of developing ookinetes and is crucial for the development of oocysts within the mosquito midgut. The infectious sporozoite stage is produced in the oocysts which leads to the subsequent transmission of malaria in a new host. Several vaccine technologies have been evaluated to design Pfs25 vaccine candidates, including recombinant proteins, viral vectors, nanoparticles, and DNA plasmids (Yusuf Y, et al. (2019) Front Immunol. 10:2412; Duffy P E. (2021) Expert Rev Vaccines. 20(2):185-198; Chichester J A, et al. (2018) Vaccine. 36(39):5865-5871; de Graaf H, et al. (2021) Front Immunol 12:694759; Kaba S A, et al. (2012) PLOS One. 7(10):e48304). Prior to the work described herein, the most advanced Pfs25 vaccine formulations have had limited success in clinical trials, eliciting weak immunogenicity and overall transmission reducing activity (Chichester J A, et al. (2018) Vaccine. 36(39):5865-5871; de Graaf H, et al. (2021) Front Immunol 12:694759; Healy S A, et al. (2021) J Clin Invest. 131(7):e146221).

Therefore, the immunogenicity of two of the leading targets, Pfs25 and PfCSP, formulated as nucleoside-modified mRNA-LNP vaccines was evaluated, and compared to Pfs25 and PfCSP DNA vaccines. As described herein, the optimal immunogenic dose and immunization schedule for eliciting functionally effective immune responses was established, including protection against sporozoite challenge and P. falciparum transmission reduction to mosquitoes. An additional critical goal of the studies described herein was to evaluate the possibility of co-immunization using multiple mRNA-LNP vaccines for immune targeting malaria parasites at various life cycle stages.

### A. Materials and Methods

### 1. Study Design.

For the initial evaluation of the mRNA-LNPs, groups (n=5) of 6-week-old to 8-week-old female Balb/c mice (Charles River) were immunized with various doses (3 μg, 10 μg, 30 μg) of either PfCSP mRNA-LNP or Pfs25 mRNA-LNP alone or were co-immunized with 10 μg of both mRNA-LNPs. In parallel, groups of Balb/c mice were also immunized with 50 μg of DNA encoding PfCSP or Pfs25 alone or a combination of both PfCSP and Pfs25.

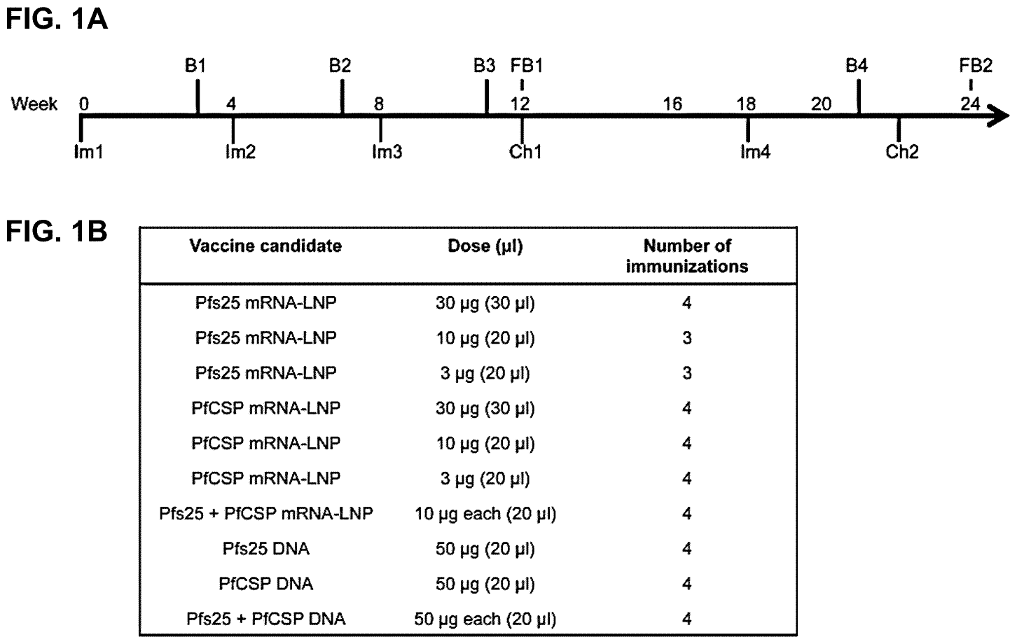

DNA plasmids were administered using in vivo electroporation with a BTX Agile Pulse electroporator (see FIG. 14A/SEQ ID NO:11 and FIG. 14B/SEQ ID NO:12). Mice were immunized with 3 or 4 doses given at 4-week intervals or as described in FIG. 1. Mice immunization schedules and timings of collection of blood and parasite challenges in various experiments are shown schematically in FIG. 1.

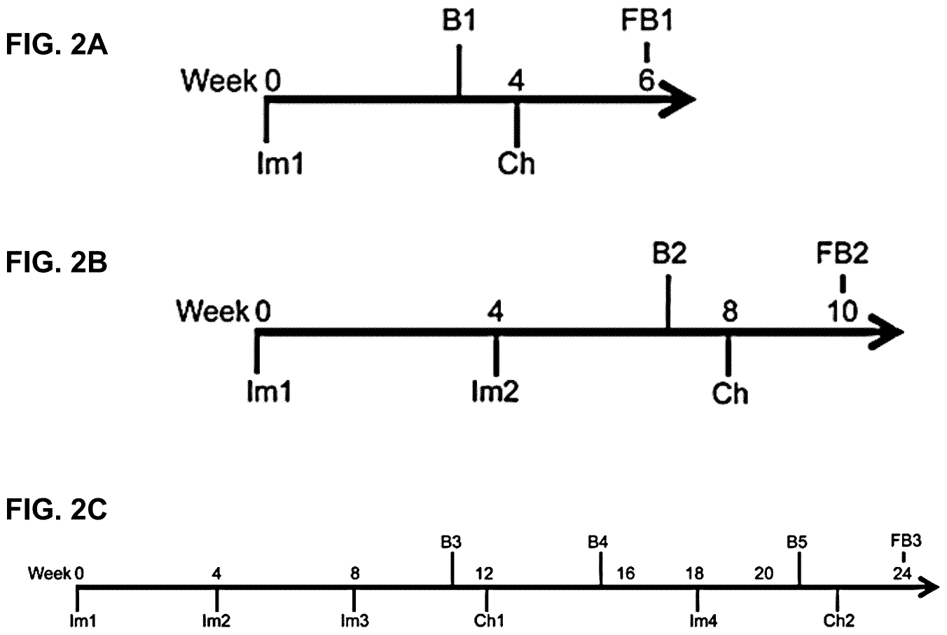

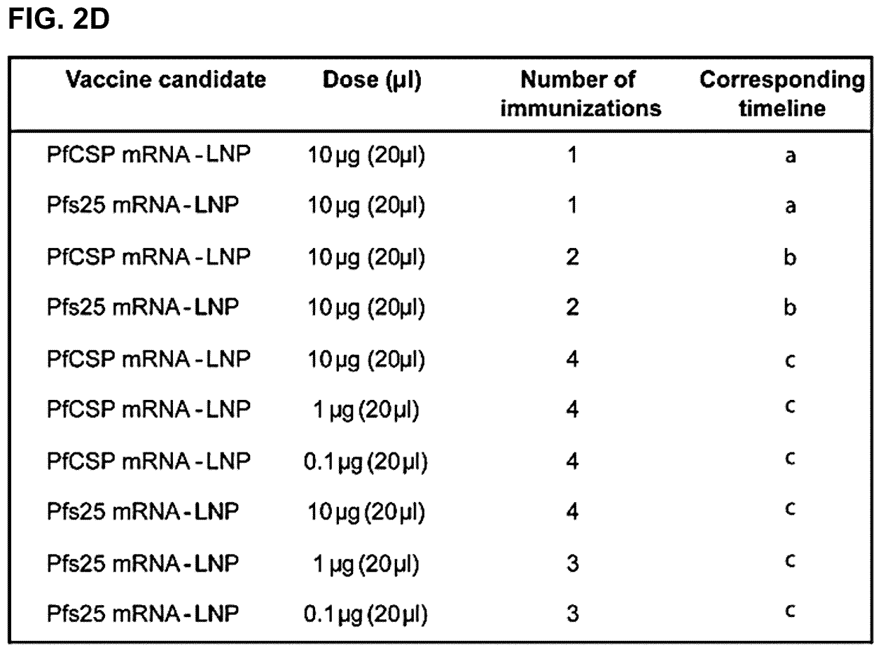

In the follow-up experiment, lower doses (0.1 μg, 1 μg, and 10 μg) and different immunization regimens (1, 2, and 3 immunizations using a fixed dose of 10 μg) of mRNA-LNPs were evaluated in groups of 6-week-old-8-week-old female Balb/c mice using immunization schedules as indicated FIG. 2A-FIG. 2C). For evaluation of cellular responses, studies included groups (n=5) of C57Bl/6 and Balb/c mice. Mice were immunized with Pfs25 mRNA-LNP, PfCSP mRNA-LNP, orrPfs25 and rPfCSP proteins formulated with the adjuvant, Alhydrogel (2% aluminum hydroxide gel suspension), and splenocytes were harvested for evaluation following either the priming dose or the subsequent booster dose administered after 15 days.

For all immunizations, mice were anesthetized with isoflurane and were immunized via intramuscular injection in the left anterior tibialis.

Typically, blood was collected three weeks following each immunization from the tail vein and at the end of each study by either cardiac puncture or retro-orbital bleed for isolation of serum for various tests (FIG. 1 and FIG. 2). In vivo experiments in mice were approved by IACUCs of the George Washington University and University of Pennsylvania. All experimentation adhered to the Guide for the Care and Use of Laboratory Animals by the National Research Council. Mice were housed and cared for in Association for Assessment and Accreditation of Laboratory Animal Care International (AAALAC)-accredited facilities.

2. Production of mRNA-LNP Vaccines.

Codon-optimized coding sequences of Pfs25 (AF193769.1) and PfCSP (XP_001351122.1) were synthesized and cloned into an mRNA production plasmid as described (Freyn A W, et al. (2020) Mol Ther. 28(7):1569-1584). Modified nucleoside containing mRNAs were produced to contain 101 nucleotide-long poly(A) tails and using m1ψ-5′-triphosphate instead of UTP. In vitro transcribed mRNAs were capped co-transcriptionally using the trinucleotide cap1 analog, CleanCap. mRNA purified by cellulose purification (Baiersdörfer M, et al. (2019) Mol Ther Nucleic Acids 15:26-35) were analyzed by agarose gel electrophoresis and were stored frozen at −20° C. until being encapsulated in LNPs using a self-assembly process. Briefly, an ethanolic lipid mixture of ionizable cationic lipid, phosphatidylcholine, cholesterol, and polyethylene glycol-lipid was rapidly mixed with an aqueous solution containing mRNA at acidic pH (Maier M A, et al. (2013) Mol Ther. 21(8):1570-1578). The LNP formulation used in this study is proprietary to Acuitas Therapeutics (U.S. Pat. No. 10,221,127). The RNA-loaded particles were characterized and subsequently stored at −80° C. at an RNA concentration of 1 mg/mL and a total lipid concentration of 30 mg/mL. The mean hydrodynamic diameter of mRNA-LNPs, measured by dynamic light scattering using a Zetasizer Nano ZS (Malvern) was ˜80 nm with a polydispersity index of 0.02-0.06 and encapsulation efficiency of 95%.

### 3. Production of DNA Vaccines.

Pfs25 DNA plasmid was constructed, as previously described (Datta D, et al. (2015) Clin Vaccine Immunol. 22(9):1013-1019), by cloning a codon-optimized (GenScript) Pfs25 sequence lacking the N-terminal signal sequence, C-terminal anchor sequence, and mutated N-linked glycosylation sites into the DNA vector VR1020 (FIG. 14B/SEQ ID NO:12). PfCSP DNA plasmid was constructed similarly by cloning a codon optimized full-length PfCSP sequence (Genscript) into the vector, VR1020 (see FIG. 14A/SEQ ID NO:11). The two plasmids were resuspended in endotoxin-free water at 2.5 mg/mL concentrations. For the co-immunization of Pfs25 and PfCSP DNA vaccines, each plasmid was concentrated, mixed in equal amounts, and diluted to a final concentration of 5 mg/L.

### 4. Parasite Challenge.

Transgenic P. berghei (ANKA) parasites expressing P. falciparum CSP, GFP, and luciferase (PbPfCSP-GFPLuc) were used for the challenges (Flores-Garcia Y, et al. (2019) Malar J. 18(1):426). Infectious sporozoites were produced by feeding Anopheles stephensi mosquitoes on an infected Swiss Webster (CFW) mouse. 20-23 days post-infection, mosquito salivary glands were dissected, placed in a 1.5 mL microcentrifuge tube containing HBSS with 2% FBS, and teased using a pestle to release sporozoites. The sporozoite suspension was filtered using a 100 μm mesh to remove debris. Mice were challenged with ˜2000 sporozoites intravenously into the tail vein. For the first challenge only, parasite liver burden was assessed by IVIS, 42 hr-44 hr post-challenge. 100 μL RediJect D-luciferin was injected intraperitoneally into mice, and the resulting bioluminescence intensity of each mouse liver was quantified using an IVIS Lumina III in vivo imaging system. Following every challenge, blood-stage parasitemia was monitored by microscopic evaluation of gimesa-stained blood smears starting three days post-challenge. Blood-stage parasitemia was monitored for at least 11 days or until 5-10% parasitemia is reached. Complete protection was defined when no parasitemia was detected for up to 11 days following the challenge. In some experiments, infected mice were administered a combination of oral sulfadiazine (30 mg/L) in drinking water and daily intraperitoneal injections of chloroquine (20 mg/kg body weight) for six days to cure mice. The clearance of parasites was confirmed by microscopic examination of gimesa-stained blood smears.

### 5. IgG Purification.

Total IgG from sera was purified using Protein-G Sepharose beads (Invitrogen). Pooled sera were incubated with beads in an equal volume of binding buffer (1.5 M glycine, 3 M NaCl, pH 9) for 3 hr-4 hr at 4° C. Beads were washed with binding buffer and the bound IgG was eluted with 0.2 M glycine pH 2.5 and collected into tubes containing 1 M Tris (pH 9) to immediately neutralize the pH. Purified IgG was concentrated to 4 mg/mL-8 mg/mL and stored at −20° C.

6. P. falciparum Gametocyte Culture.

Mature gametocytes of the human malaria parasite, P. falciparum (NF54), were cultured as described earlier (Trager W, et al. (1976) Science 193(4254):673-675; Tripathi A K, et al. (2020) J Vis Exp. (161):10.3791/61426). Parasites were cultured using O+ human erythrocytes at 4% hematocrit in parasite culture medium (RPMI 1640 supplemented with 25 mM HEPES, 10 mM glutamine, 0.074 mM hypoxanthine, and 10% O+ human serum). Gametocyte cultures were initiated at 0.5% parasitemia from low-passage stock and were maintained up to day 18 with daily medium changes. Culture plates were incubated at 37° C. in a microaerophilic environment inside a candle jar. The use of human erythrocytes to support the growth of P. falciparum was approved by the Internal Review Board (IRB) of the Johns Hopkins University Bloomberg School of Public Health (#NA 00019050).

### 7. Standard Membrane Feeding Assay (SMFA).

The functional activity of transmission-blocking vaccines was evaluated by SMFA as described previously (Datta D, et al. (2015) Clin Vaccine Immunol. 22(9):1013-1019). Purified IgG was diluted to desirable test concentrations in a mixture of approximately 0.3% P. falciparum (NF54) gametocytes, human red blood cells (50% hematocrit), and normal human sera. The antibody-parasite mixture was fed to 25-40, 4-day old to 6-day old Anopheles stephensi mosquitoes (starved 6 hrs to 8 hrs) using glass membrane feeders maintained at 37° C. for 15 min.

Unfed mosquitoes were removed, and blood-fed mosquitoes were maintained at 27° C.-28° C. with 70-80% relative humidity for 7 days to 8 days. Mosquito midguts were dissected, and oocysts were enumerated after staining with 0.5% mercurochrome for 15-20 min. Transmission reducing activity is defined as the percent reduction in mean oocysts between the test IgG-fed mosquitoes and the control IgG-fed mosquitoes. Transmission blocking activity is defined as the percent reduction in the proportion of infected mosquitoes between the test IgG fed and control IgG fed groups.

### 8. Recombinant Pfs25 and PfCSP and Synthetic Peptides.

Recombinant Pfs25 protein was purified as reported previously (Kumar R, et al. (2014) Infect Immun. 82(4):1453-1459). Recombinant PfCSP (SEQ ID NO:13) was produced by expression in Escherichia coli. Codon harmonized PfCSP sequence (lacking N-terminal signal and C-terminal GPI anchor sequence) with a 6× histidine tag fused at the 5′ end with a spacer (PGGSGSGT) (SEQ ID NO:31) was synthesized (GenScript) and cloned into a pET (K-) expression vector and transformed into BL21 (DE3) E. coli. Protein expression was induced with 0.1 mM IPTG. Bacteria were harvested by centrifugation (Beckman Avanti J-E, JLA9.1) at 7460×g for 30 min at 4° C. The cell pellet was resuspended in lysis buffer (50 mM NaH₂PO₄, 300 mM NaCl, 20 mM imidazole pH 8) and lysed via microfluidization. Lysed bacteria were centrifuged (Beckman Avanti J-E, JA-25.50) at 20,442×g for 30 min at 4° C., and the supernatant was clarified by filtration (0.45 um) and incubated with Ni-NTA beads (Qiagen) at 4° C., overnight. Ni-NTA beads were washed with the wash buffer (50 mM NaH₂PO₄, 300 mM NaCl, 20 mM imidazole pH 8) containing 0.25% Sarkosyl followed by the wash buffer alone. Bound protein was eluted from the beads using 250 mM imidazole in phosphate buffer (50 mM NaH₂PO₄, 300 mM NaCl, pH 8). Buffer exchange with PBS pH 7.4 was conducted using Amicon Ultra centrifugal filters (30 kDa MWCO) and rPfCSP was stored at −20° C. Seventeen overlapping synthetic peptides (20 amino acids long with a 10 amino acid overlap-SEQ ID NO: 14-SEQ ID NO:30) of Pfs25 (amino acids 18-210) (SEQ ID NO:01) were synthesized by Biomatik. Peptides were supplied as TFA salt and estimated to be >85% pure.

### 9. Enzyme-Linked Immunosorbent Assay (ELISA).

PfCSP and Pfs25-specific antibodies were analyzed by ELISA using Nunc MaxiSorp 96 well plates coated with 100 ng/ml rPfCSP in DPBS overnight at 4° C., and Immulon 4hbx 96 well plates coated with 1 μg/mL rPfs25 in 0.1 M carbonate-bicarbonate buffer (pH 9.6), respectively.

Pfs25-coated plates were blocked with 1% BSA and 0.1% Tween-20 in DPBS, while PfCSP-coated plates were blocked with 1% BSA in DPBS. For endpoint titer analysis, sera, in duplicate, were diluted in corresponding blocking buffers and incubated for 1 hr at 25° C. Plates were washed; first with DPBS containing 0.5% Tween-20 followed by DPBS alone. Plates were incubated with 1:2000 dilution of peroxidase-conjugated goat anti-mouse IgG (SeraCare) secondary antibody for 1 h at 25° C., washed as above, and developed using a one-component ABTS substrate (SeraCare). After development, the enzyme reaction was stopped with 1% SDS and plates were read at 405 nm by VersaMax ELISA reader (Molecular Devices). Endpoint titers were determined using the average plus three standard deviations of the optical densities (OD) of pooled pre-immune mouse sera replicates as a cutoff. Antigen-specific antibody isotypes were evaluated for each group's pooled serum at a dilution corresponding to the absorbance values within the linear range of the ELISA reaction, in duplicates. Peroxidase labeled goat anti-mouse IgG1, IgG2a, IgG2b, and IgG3 (Southern Biotech) were used at 1:5000 dilution, and the plates were processed as above. Antibody-antigen avidity was evaluated using sera at fixed dilutions corresponding to the absorbance values within the linear range of the ELISA reaction in duplicates. Sera were incubated at 37° C. for 1 hr followed by incubation with various concentrations of NaSCN (0 M, 1 M, 2 M, 4 M, 8 M) for 15 min. Washed plates were incubated with peroxidase-labeled goat anti-mouse IgG for 1 hr at 37° C. and developed as above. Avidity index was defined as the NaSCN concentration resulting in 50% dissociation of antibody-antigen binding.

### 10. Production of Fluorescently-Labeled Proteins.

Recombinant PfCSP and Pfs25 protein were fluorescently labeled as previously described (Alameh M G, et al. (2021) Immunity. 54(12):2877-2892.e7). Briefly, PfCSP and Pfs25 were independently conjugated to either PE or AlexaFluor 647 using the Lightning-Link R-Phycoerythrin (R-PE) and Lightning-Link I Rapid Alexa Fluor 647 according to the manufacturer's instructions (Novus Biologicals). Adjustment to the molar ratio was performed as per manufacturer recommendations.

### 11. Flow Cytometry Analysis of Memory B and GC B Cells.

Spleens were collected and processed using 40 μm cell strainers in complete DMEM to obtain single-cell suspensions. All steps were carried out at 4° C. RBCs were first lysed with ACK (5 minutes), then splenocytes were washed twice and incubated with fluorescently labeled PfCSP or Pfs25. All cells were stained for live-dead, incubated with Fc block (Biolegend) for 20 minutes at 4° C., washed with FACS buffer (1% BSA in PBS), and stained for 1 hr using the antibody panel in Table 2.

TABLE 2
Detailed List of Antibodies Used for
B Cell Analysis by Flow Cytometry

| Marker | Clone | Dilution | Catalog # |
|---|---|---|---|
| B220 | RaA3-6B2 | 1:200 | 103225 |
| IgD | 11-26c.2a | 1:300 | 405725 |
| Aqua | n/a | 1:400 | 423102 |
| CD19 | 6D5 | 1:100 | 115543 |
| CD38 | 90 | 1:100 | 56-0381-82 |
| IgM | R6-60.2 | 1:200 | 562565 |
| CD4 | H129.19 | 1:800 | 130312 |
| CD8 | 53-6.7 | 1:400 | 553034 |
| Gr-1 | RB6-8C5 | 1:800 | 108410 |
| F4/80 | BM8 | 1:800 | 15-4801-82 |
| GL7 | GL7 | 1:200 | 144620 |

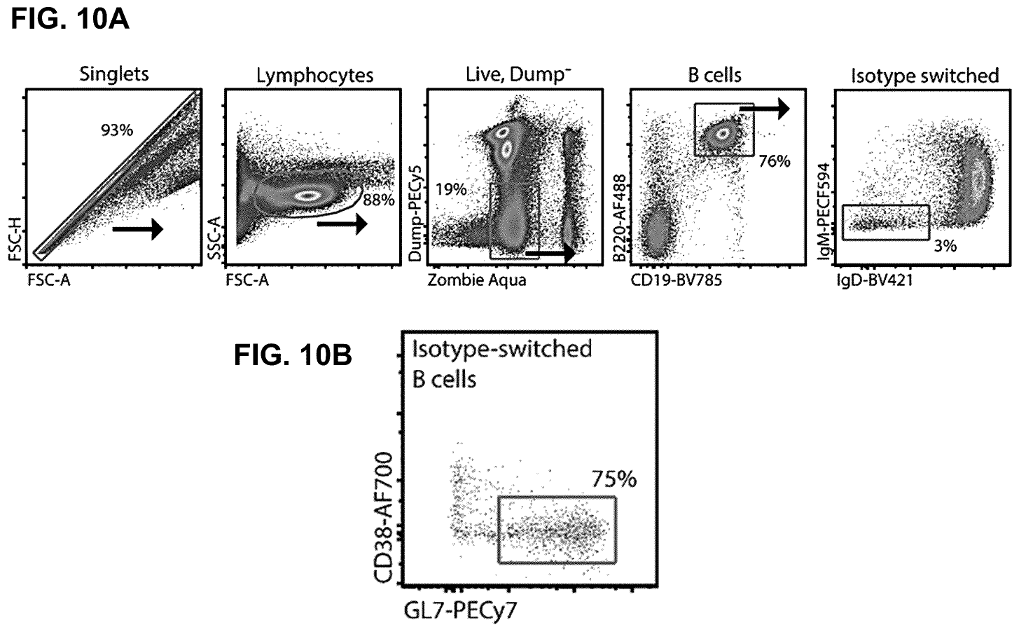

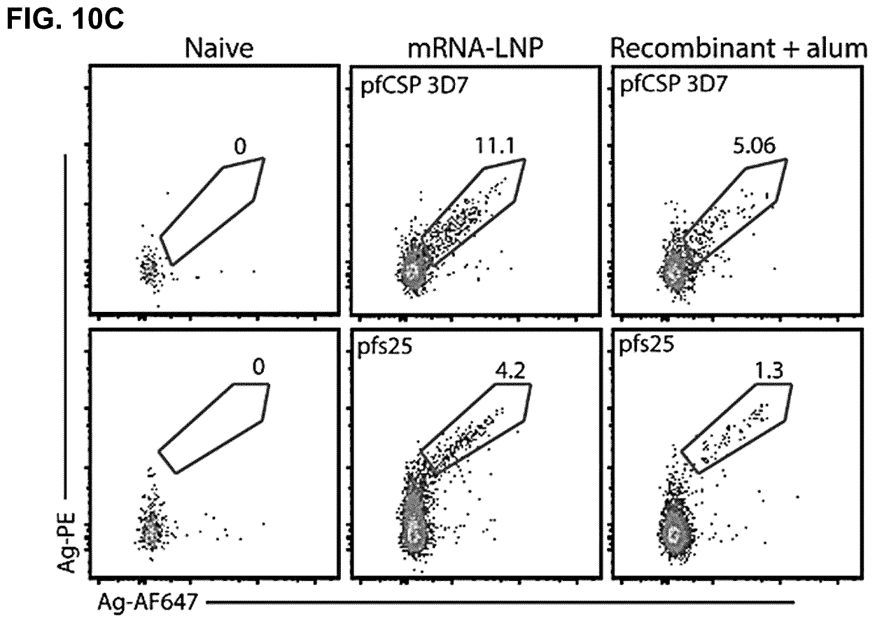

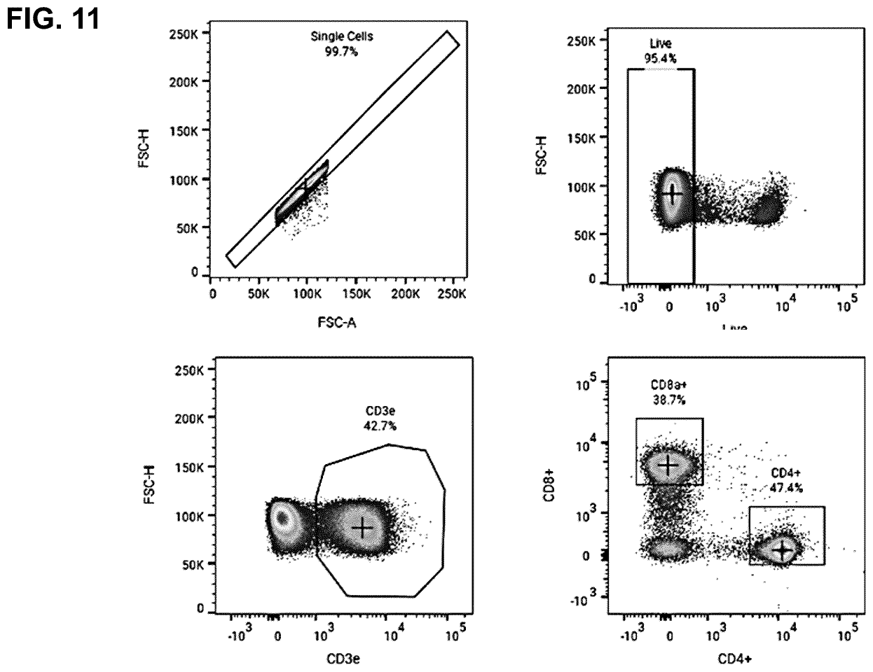

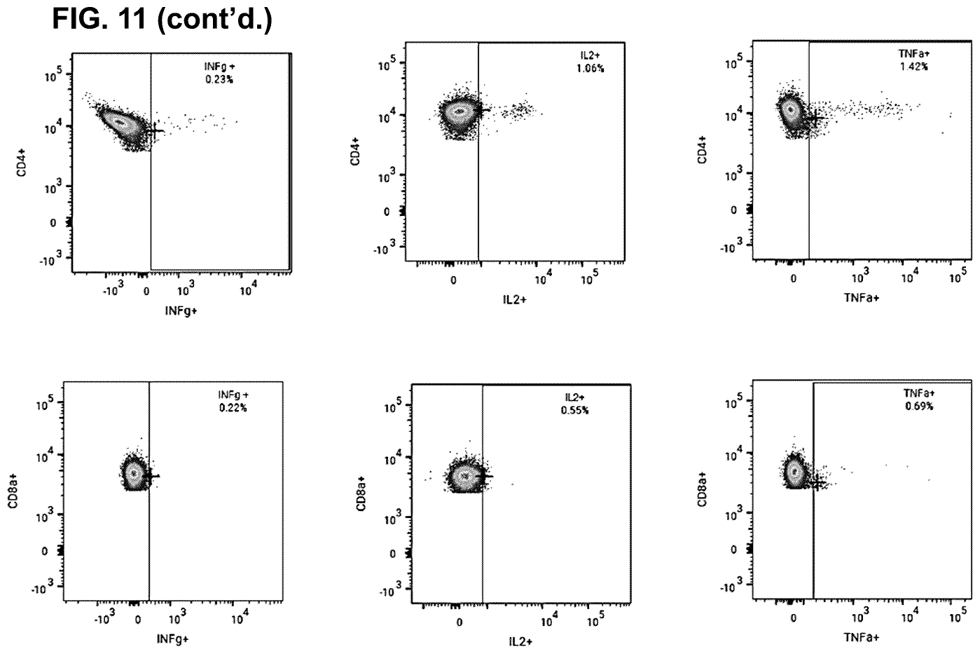

Following staining, cells were washed twice, fixed with 300 μL (1% paraformaldehyde) and samples were acquired on a BD LSR II equipped with 4 laser lines and 18 PMTs. The gating strategy is provided in FIG. 10A-FIG. 10C.

### 12. Flow Cytometry Analysis of T Cells.

Splenocytes were collected, counted using a Vi-Cell automated cell counter, resuspended at 20,000 cells/μL, and seeded into a 5 mL polypropylene FACS tube (100 μL/tube). For Pfs25 restimulation, cells were stimulated with 80 μl of a pool of 17 Pfs25 synthetic peptides at a final concentration of 2.5 μg/mL, and 1 μg/mL of CD28/CD49d mixture (co-stimulatory signal) for 6 hours. One hour post-stimulation a volume of 20 μL containing brefeldin-A and monensin at final concentrations of 0.2 and 0.14 μM, respectively, was added to the cells to block cytokine secretion. Cells were then washed using PBS, stained for live-dead cells using the Aqua LD stain (Thermo Fisher), and blocked for 20 minutes in the dark using Fc block (Biolegend) before staining with specific antibodies. Splenocytes were stained as per antibody panel in Table 3. Samples were acquired on a BD LSR II equipped with 4 laser lines and 18 PMTs. The gating strategy is provided in FIG. 2.

TABLE 3
Detailed list of antibodies used for
T cell analysis by Flow Cytometry

| Marker | Clone | Dilution | Catalog # |
|---|---|---|---|
| CD107a | 1D4B | 1:100 | 553793 |
| CD4 | GK1.5 | 1:200 | 100434 |
| CD8a | 53-6.7 | 1:200 | 100725 |
| L/D | | 1:200 | L34966 |
| CD3e | 145-2C11 | 1:200 | 100351 |
| IL-4 | 11B11 | 1:200 | 564006 |
| IL-2 | | 1:200 | 554429 |
| IFN-g | XMG1.2 | 1:200 | 557998 |
| IL-17 | TC11-18H10.1 | 1:200 | 506940 |
| IL-5 | TRFK5 | 1:200 | 504304 |
| TNF-a | MP6-XT22 | 1:200 | 557644 |

### 13. Statistical Analysis.

All statistical analyses were performed using GraphPad Prism software. Two-sided Mann-Whitney U test was used to analyze antibody titers and SMFA data. The log-rank (Mantel-Cox) test was used to analyze survival curves. One way ANOVA was used to analyze avidity data. All statistical tests were conducted using a 5% significance level.

### B. Specific Examples

### Example 1

Pfs25 mRNA-LNPs Elicited High Antibody Titers in a Dose-Dependent Fashion

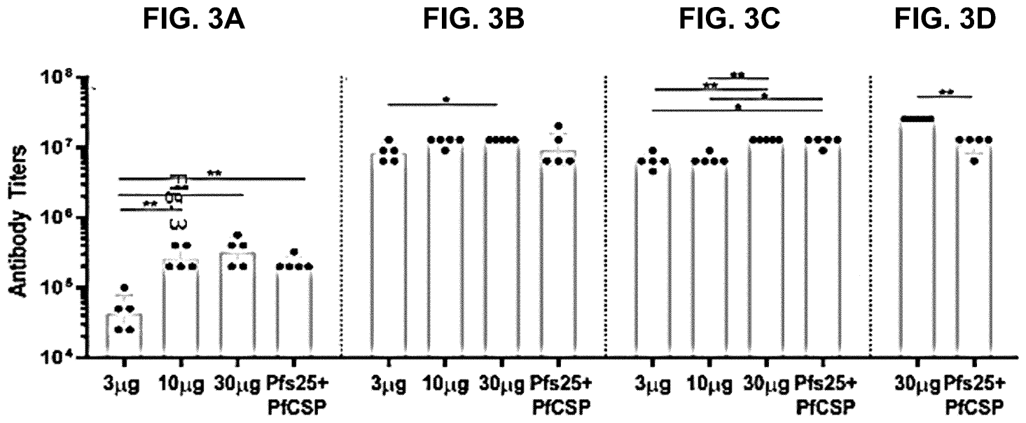

To assess the antibody responses to Pfs25, female Balb/c mice were immunized with various doses (3 μg, 10 μg, and 30 μg) of Pfs25 mRNA-LNP and evaluated as shown in FIG. 1A. After the first immunization, the 3 μg, 10 μg, and 30 μg Pfs25 mRNA-LNPs elicited dose-dependent primary antibody responses with geometric mean titers of 43,528, 263,902, and 324,901, respectively (FIG. 3A). Administration of the second immunization of Pfs25 mRNA-LNP resulted in significant boosting of antibody responses with titers ˜200-fold higher for the lowest dose (ELISA titer 8,444,851) and ˜20-fold higher for the 10 μg (ELISA titer 11,942,822) and 30 μg (ELISA titer 12,800,000) Pfs25 mRNA-LNP doses (FIG. 3B). Additional immunization did not result in any further increase in antibody titers (FIG. 3C-FIG. 3D). While all doses elicited strong antibody production, the 30 μg Pfs25 mRNA-LNP elicited significantly higher titers compared with the 3 μg (p=0.0079) and 10 μg (p=0.0079) Pfs25 mRNA-LNP groups after three immunizations.

### Example 2

PfCSP mRNA-LNPs Elicited High Antibody Titers in a Dose-Dependent Fashion

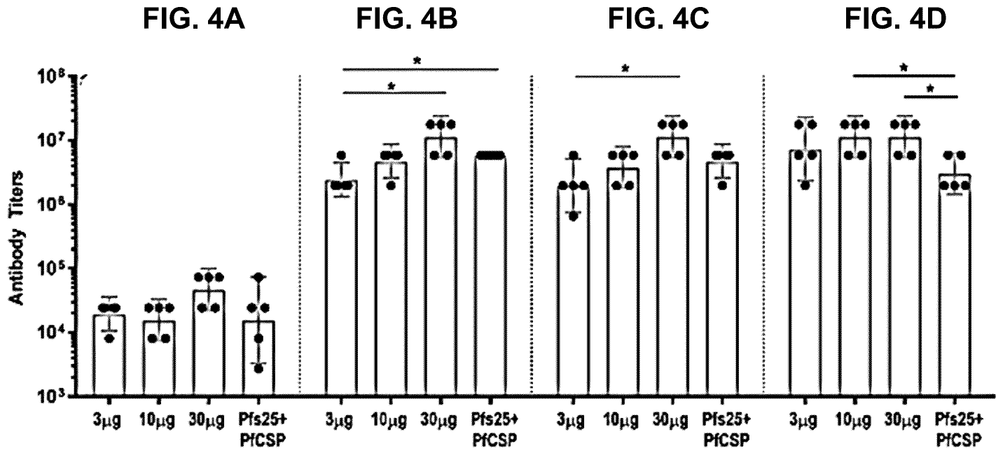

PfCSP mRNA-LNPs was evaluated at doses of 3, 10, and 30 μg, as shown in FIG. 1A. After one immunization, the 3 μg, 10 μg, and 30 μg PfCSP mRNA-LNP groups elicited PfCSP-specific antibody responses with geometric mean titers of 19,507, 15,658, and 46,976 (FIG. 4A). A second immunization resulted in 100 to 200-fold increases in antibody titers, with geometric means reaching 2,451,972, 4,740,109, and 11,415,247 for the 3 μg, 10 μg, and 30 μg PfCSP mRNA-LNP groups, respectively (FIG. 4B). Although there was a clear trend of a dose-dependent increase in the immunogenicity of PfCSP mRNA-LNP, the only statistically significant difference was between the 3 μg and 30 μg PfCSP mRNA-LNP groups (p=0.0238). Any further immunization did not significantly alter antibody titers (FIG. 4C), with observed geometric mean titers of U.S. Pat. Nos. 1,968,300, 3,805,082, and 11,415,247 for the 3 μg, 10 μg, and 30 μg PfCSP mRNA groups, respectively.

As discussed below, the protection against sporozoite challenge after three immunizations was evaluated; however, the purified sporozoites used for challenge were found to be largely non-viable thus necessitating another challenge after a period of drug treatment and a fourth immunization (FIG. 1A). Geometric mean antibody titers after a fourth immunization in the 3 μg and 10 μg PfCSP mRNA-LNPs groups increased to U.S. Pat. Nos. 7,355,917 and 11,415,247, with the 30 μg mRNA-LNP group remaining at similar titers to those prior to the fourth immunization (FIG. 4D).

### Example 3

Co-Immunization with Pfs25 and PfCSP mRNA-LNP Did not Compromise Antibody Responses

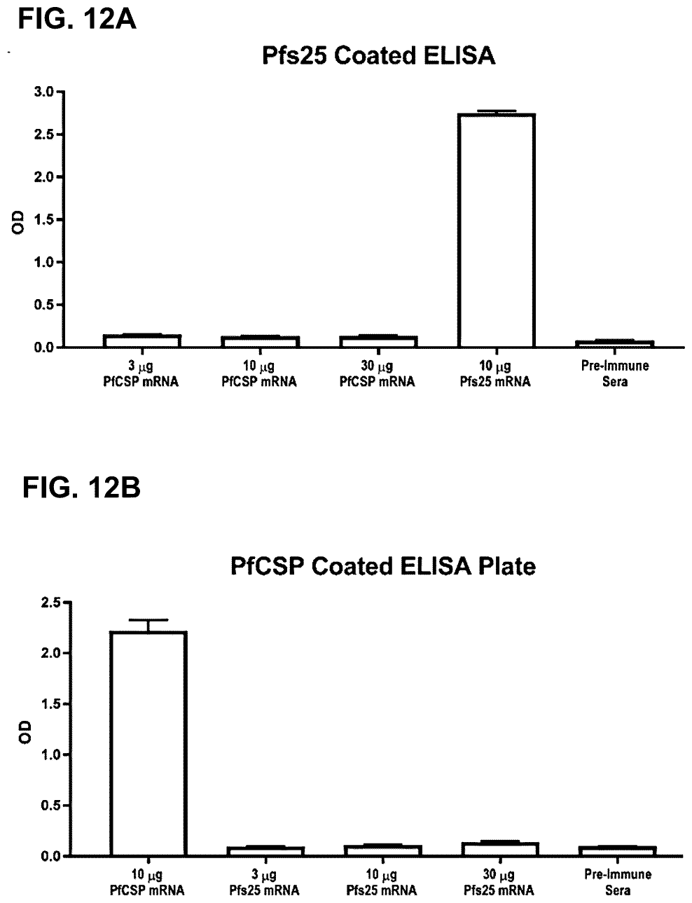

A group of mice was immunized with a combination of both Pfs25 and PfCSP mRNA-LNPs to explore the immunogenicity of co-immunization of antigens targeting different parasite life cycle stages. Female Balb/c mice were co-immunized with 10 μg of Pfs25 and PfCSP mRNA-LNPs (Pfs25+PfCSP mRNA-LNP), and the individual antigen-specific antibody titers were compared with antibody titers in mice immunized with Pfs25 mRNA-LNP or PfCSP mRNA-LNP alone. As shown in FIG. 3 and FIG. 4, Pfs25 and PfCSP-specific antibody titers after each immunization in the co-immunized group remained largely comparable to those obtained in mice immunized with either of the two immunogens individually. The only notable difference was higher Pfs25-specific titers after three immunizations in the combination group as compared to 10 μg Pfs25 mRNA-LNP (FIG. 3; p=0.0079). Surprisingly, in the case of PfCSP-specific antibody responses, mice that had undergone experimental manipulations consisting of a challenge with largely non-viable sporozoites and drug treatment showed somewhat lower antibody titers after the fourth immunization in the combination group as compared to 10 μg PfCSP mRNA-LNP group (FIG. 4D). To rule out the possibility of any cross-reactivity between the two antigens, sera from mice immunized with Pfs25 mRNA-LNPs was tested for reactivity with PfCSP and vice versa using ELISAs. As shown (FIG. 12A-FIG. 12B), immunized mice sera displayed antigen specific reactivity while the reactivity of Pfs25 immunized mice sera to PfCSP and the reactivity of PfCSP immunized mice sera to Pfs25 was comparable to that of pre-immune mouse sera used as a negative control for all ELISA.

### Example 4

mRNA-LNPs Elicited Superior Antibody Responses than Electroporation-Mediated DNA Plasmids

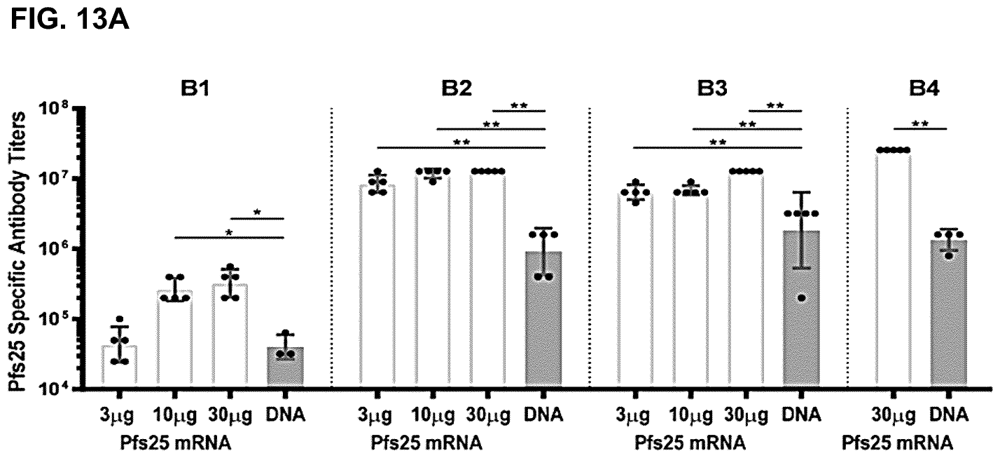

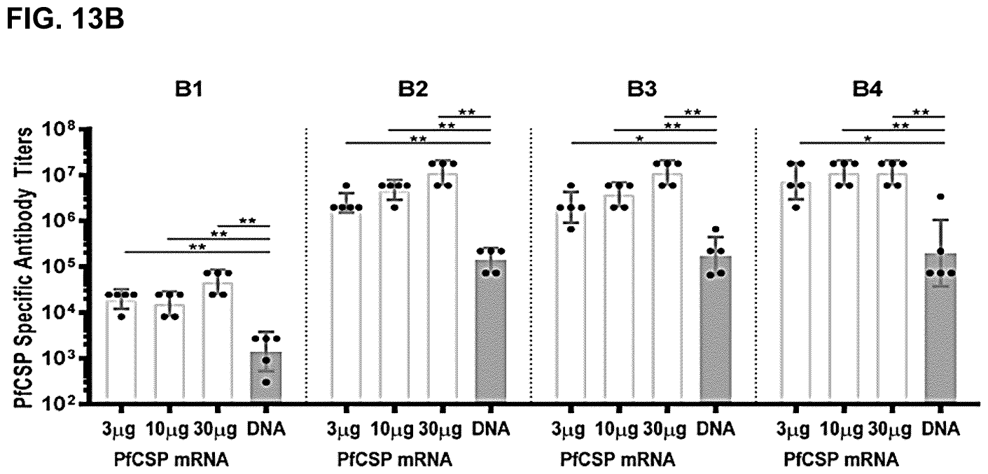

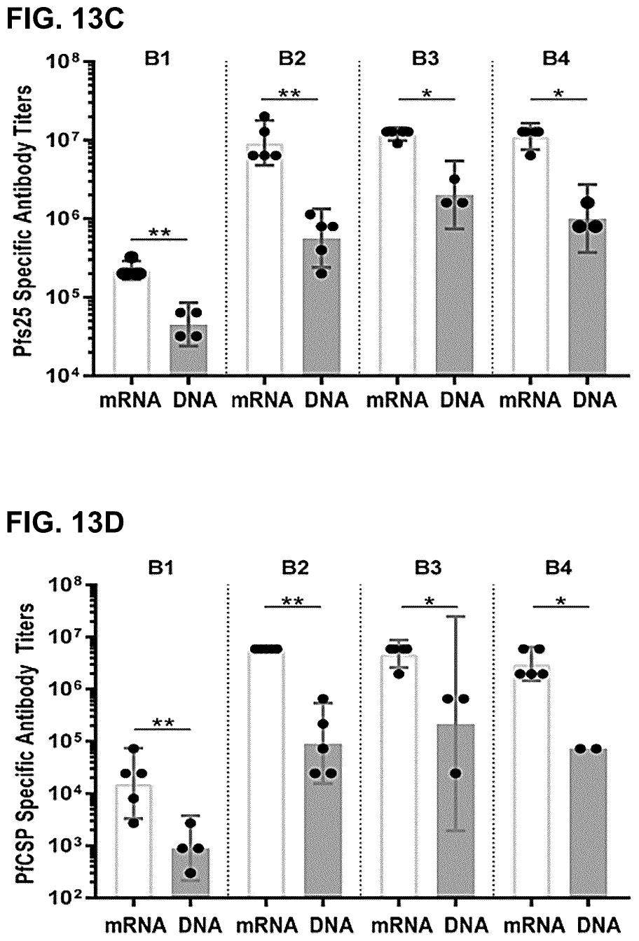

Previously, 50 μg of Pfs25 DNA administered with electroporation (EP) was shown to be immunogenic and efficacious in mice (Datta D, et al. (2015) Clin Vaccine Immunol. 22(9):1013-1019). Another goal of these studies was to compare the relative immunogenicity differences between mRNA-LNP and DNA with in vivo EP for both Pfs25 and PfCSP. After each immunization, the antibody titers elicited by Pfs25 and PfCSP mRNA-LNPs, regardless of the vaccine dose, were superior to those elicited by immunization with Pfs25 and PfCSP DNA vaccines administered using in vivo EP (FIG. 13A-FIG. 13B). Likewise, Pfs25 and PfCSP mRNA-LNPs combination (10 μg each) vaccine-elicited antibody responses were also superior as compared to antibodies elicited in mice co-immunized with a combination of Pfs25 and PfCSP DNA (25 μg each) administered with EP (FIG. 13A-FIG. 13D).

### Example 5

Pfs25 mRNA-LNPs Induced Potent Transmission-Blocking Antibodies

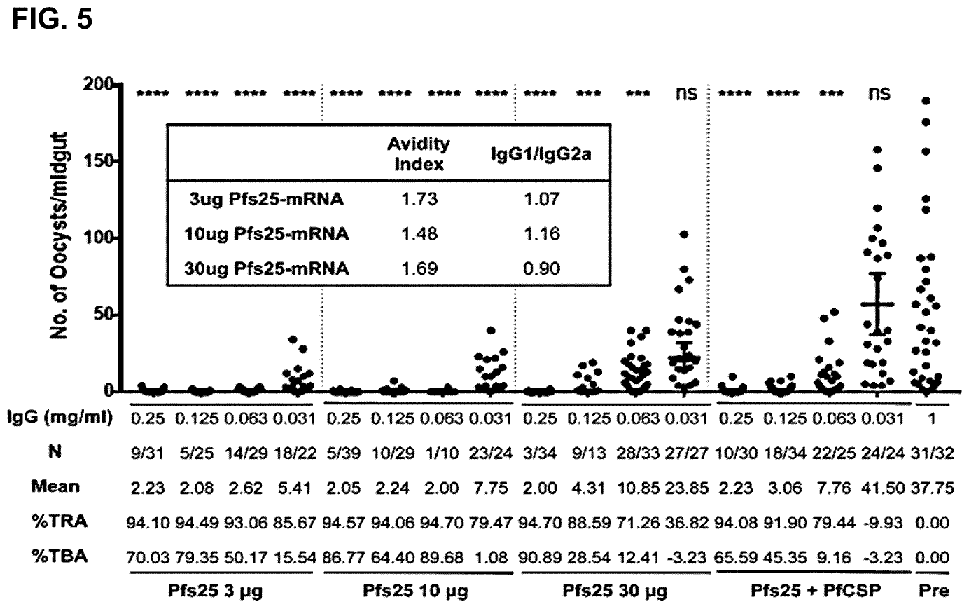

Due to the limited volume of sera collected, serum collected from final bleeds from each mouse was pooled to purify IgG for evaluation in SMFA. Sera (FB1 in FIG. 1A) from mice immunized three times with 3 μg and 10 μg Pfs25 mRNA-LNP were pooled and evaluated, while the 30 μg Pfs25 mRNA-LNP and (Pfs25+PfCSP) mRNA-LNP combination groups were evaluated after four immunizations (FB2 in FIG. 1A). Initially, purified IgG from all the groups of the 3 μg, 10 μg, and 30 μg Pfs25 mRNA-LNP and Pfs25+PfCSP mRNA-LNP groups were evaluated at three concentrations (2 mg/mL, 1 mg/mL, 0.5 mg/mL) and all revealed greater than 94% TRA at all three concentrations of IgG tested. In subsequent experiments, lower concentrations of IgG (0.25 mg/mL, 0.125 mg/mL, 0.0625 mg/mL, 0.03125 mg/mL) were evaluated. IgG purified from pooled pre-immune mouse sera was used at 1 mg/mL as a negative control. IgG purified from the 3 μg and 10 μg Pfs25 mRNA-LNP immunized mice achieved greater than 90% oocyst reduction (TRA) at IgG concentrations as low as 0.0625 mg/mL. Surprisingly, IgG from the 30 μg Pfs25 mRNA-LNP and Pfs25+PfCSP combination groups revealed relatively lower reduction with TRA>90% at 0.125 mg/mL to 0.25 mg/ml IgG concentrations (FIG. 5).

To further characterize the anti-Pfs25 antibodies of sera that have transmission-blocking activity, Pfs25-specific antibody avidity and isotypes were evaluated using pooled serum of terminal bleeds of each group by ELISA (FIG. 5). The avidity indices of 1.73, 1.48, and 1.69 were similar among the pooled sera of the 3 μg, 10 μg, and 30 μg Pfs25 RNA-LNP immunized mice, respectively. Pfs25-specific IgG isotypes (expressed as IgG1/IgG2a ratios) were compared in these sera and the results revealed balanced IgG isotypes with IgG1/IgG2a ratios of 1.20, 1.07, and 1.17 for the 3 μg, 10 μg, and 30 μg Pfs25 mRNA-LNP groups, respectively.

### Example 6

Protection Against Sporozoite Challenge after Immunization with PfCSP mRNA-LNP

The protection provided by the immunization with PfCSP was evaluated using an in vivo challenge model using sporozoites of transgenic P. berghei expressing PfCSP and luciferase (PbPfCSP-GFPLuc). For the first challenge (Ch1 in FIG. 1A), mice were inoculated with ˜2000 PbPfCSP-GFPLuc sporozoites four weeks after the third immunization. Liver stage parasite burden was evaluated 42 hr-44 hf after the challenge by IVIS, quantifying the subsequent bioluminescence from the liver. None of the challenged mice, including negative controls, had any detectable liver stage burden, which indicates that the sporozoites used for the challenge were not very viable or infectious. Additionally, only a fraction of Pfs25 immunized control mice (6/10) had any detectable blood-stage parasites by day 7 further corroborating that the viability of PbPfCSP-GFPLuc sporozoites was severely compromised during sporozoite isolation.

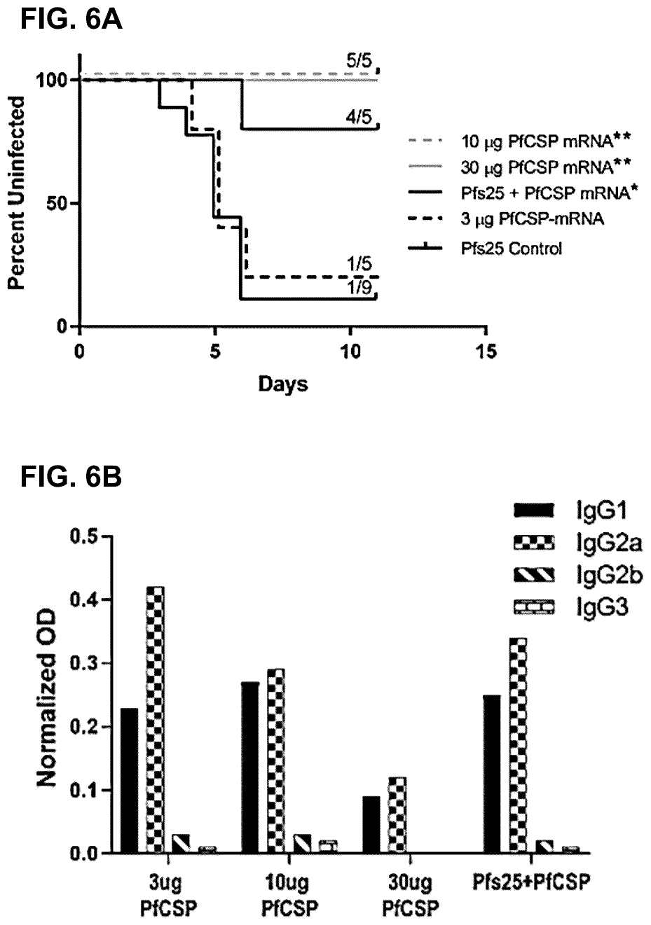

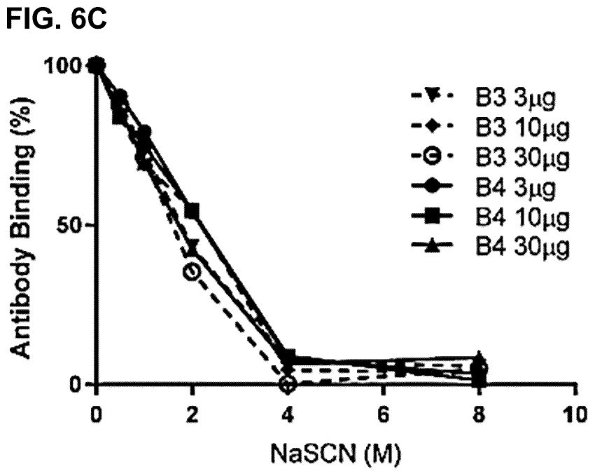

To further evaluate the protective efficacy of the PfCSP mRNA LNP vaccine, mice were treated with chloroquine and sulfadiazine for six days to treat for any low-level parasitemia. Mice were given a fourth immunization after a rest period, and four weeks after the last dose of immunization mice were challenged again (Ch2 in FIG. 1A) and monitored for blood-stage parasitemia for 11 days. Five out of five mice of each 10 μg and 30 μg PfCSP mRNA-LNP group and four out of five mice of the Pfs25+PfCSP mRNA-LNP group were fully protected. In contrast, the 3 μg PfCSP mRNA-LNP group only had one out of five mice that achieved complete protection. Kaplan-Meier survival curves representing the percent of mice with detectable parasitemia each day following the challenge were used to assess any significant delays in blood-stage parasitemia. The survival curves of the 10 μg PfCSP mRNA-LNP (p=0.004), 30 μg PfCSP mRNA-LNP (p=0.004), and Pfs25+PfCSP mRNA-LNP groups (p=0.0132), were significantly different to the Pfs25 mRNA-LNP immunized groups which served as a negative control for protection against infection by sporozoites. In contrast, the 3 μg PfCSP mRNA-LNP group showed no significant difference when compared to the Pfs25 mRNA-LNP control (p=0.8319) (FIG. 6A).

To further characterize antibody responses at the time of protection, PfCSP-specific antibody avidity and isotypes were evaluated on pooled sera collected three weeks after the fourth dose (FIG. 6). The PfCSP-specific antibody avidity indices were similar between 3 μg, 10 μg, and 30 μg PfCSP mRNA-LNP groups, with avidity indices of 2.18, 2.20, and 1.72, respectively. To rule out influence of any potential impact of the transgenic parasites used in the first non-viable challenge, avidity of sera collected one week prior to the first challenge (B3 in FIG. 1A) was also assessed. The avidity indices for the 3 μg, 10 μg, and 30 μg PfCSP mRNA-LNP groups were 1.77, 2.01 and 1.59, respectively, which are within the experimental variability of the assay and similar to the avidity of sera collected three weeks after the fourth dose (B4 in FIG. 1A). In addition, the immune sera revealed IgG2a biased antibody isotypes with significantly pronounced IgG2a skewed isotypes in the sera from mice immunized with 3 μg and 30 μg PfCSP mRNA-LNP and the combination of Pfs25+PfCSP mRNA-LNP, with IgG1/IgG2a ratios of 0.75, 0.54, and 0.73, respectively.

### Example 7

Evaluation of the Minimum Effective Dose of Pfs25 mRNA-LNP and PfCSP mRNA-LNP

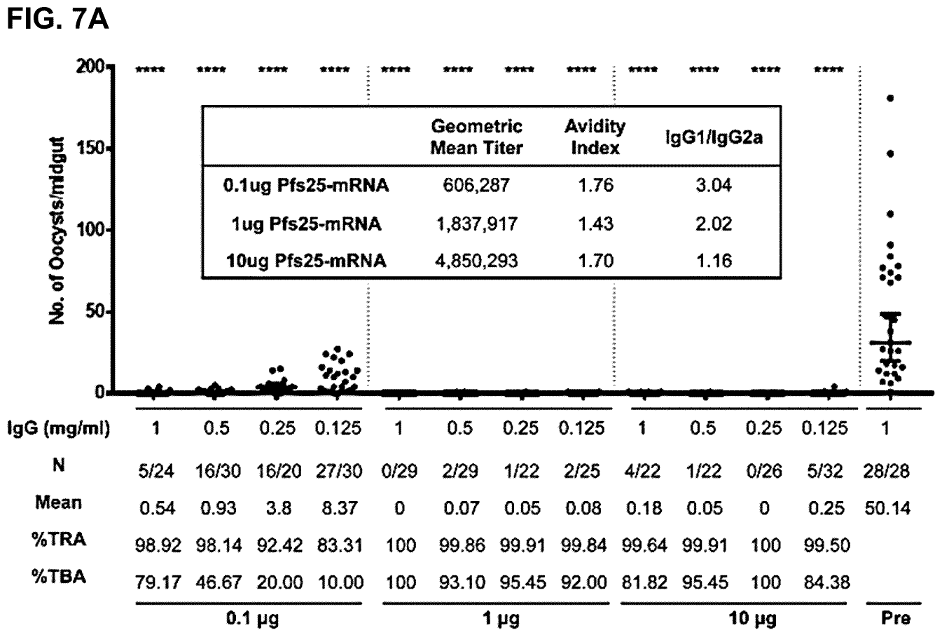

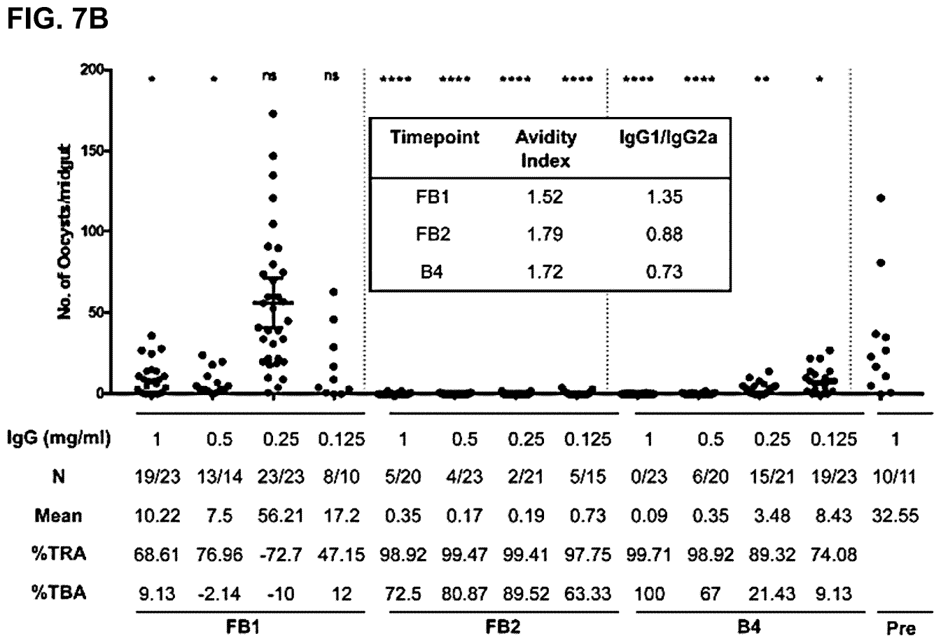

Due to the significant antibody responses elicited by the 3 μg mRNA-LNP group, whether lower doses of 1 μg or 0.1 μg mRNA-LNP would be immunogenic with effective antibody responses was also investigated. Mice immunized three times (FIG. 2C) with 0.1 μg, 1 μg, and 10 μg Pfs25 mRNA-LNP revealed a dose-dependent antibody response with geometric mean titers of 606,287, 1,837,917, and 4,850,293, respectively (FIG. 7A-inset). The 1 μg Pfs25 mRNA-LNP group had higher antibody titers than the 0.1 μg Pfs25 mRNA-LNP group, but the difference was not statistically significant. However, the antibody titers in the mice immunized with 10 μg Pfs25 mRNA-LNP were significantly higher than the 1 μg (p=0.0476) and 0.1 μg (p=0.0079) Pfs25 mRNA-LNP groups.

SMFA was conducted to evaluate the functional efficacy of the antibodies elicited by lower doses of Pfs25 mRNA-LNP (FIG. 7). Purified IgG was evaluated at 1.0 mg/mL, 0.5 mg/mL, 0.25 mg/mL, and 0.125 mg/mL final concentrations, with IgG purified from pre-immune sera used as a negative control at 1 mg/mL. The IgG purified from the 1 μg and 10 μg Pfs25 mRNA-LNP immunization groups elicited nearly 100% blocking at the lowest concentration of 0.125 mg/ml IgG tested. IgG from the 0.1 μg Pfs25 mRNA-LNP immunized mice, appeared to be relatively less potent, eliciting 98-99% TRA only at higher (0.5 to 1.0 mg/mL) IgG concentrations, with decreasing TRA at lower concentrations (<0.25 mg/mL) of IgG tested.

The antibody isotype and avidity in the sera used for the SMFA (FIG. 7a, inset). The avidity indices for the 0.1 μg, 1 μg, and 10 μg groups were not different from those seen earlier when higher immunizing doses up to 30 μg were evaluated (FIG. 5). Strikingly, the antibody isotype analysis did reveal rather unexpected IgG1 skewed responses with IgG1/IgG2a of 3.04, and 2.02 in the mice immunized with 0.1 μg and 1.0 μg Pfs25 mRNA-LNP, respectively. Consistent with the previous analysis, the 10 μg Pfs25 mRNA-LNP group elicited a balanced response, with an IgG1/IgG2a ratio of 1.16.

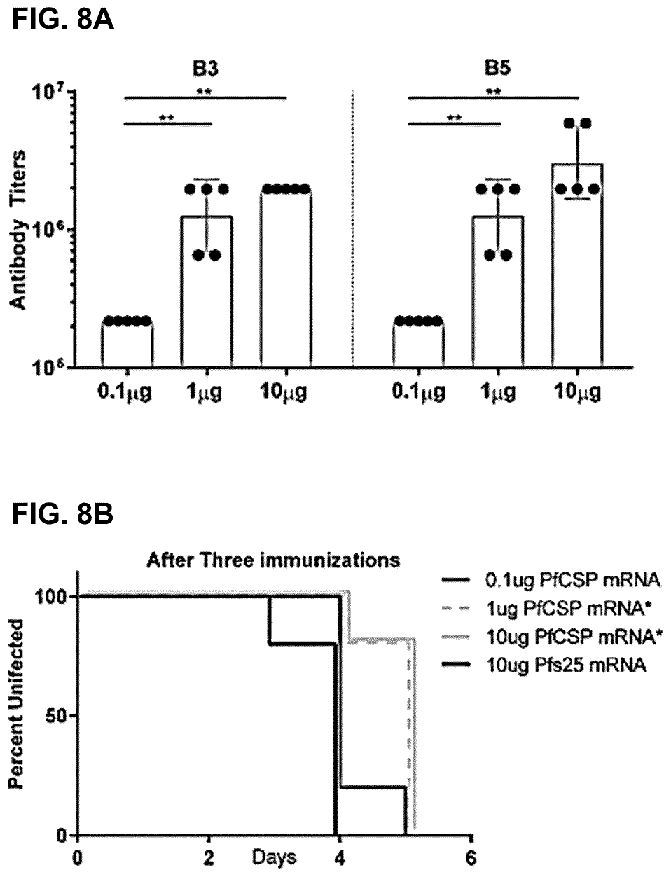

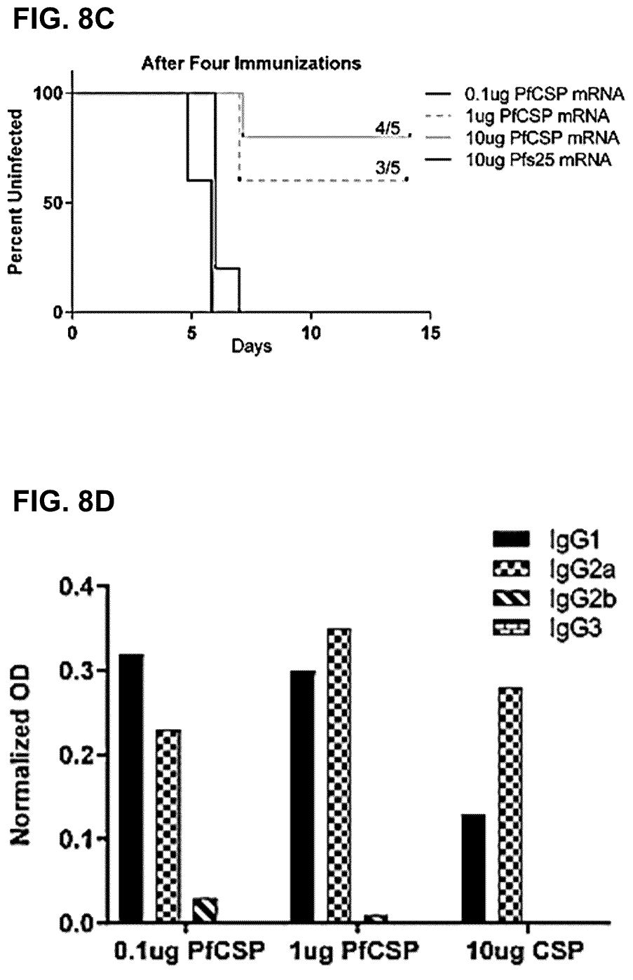

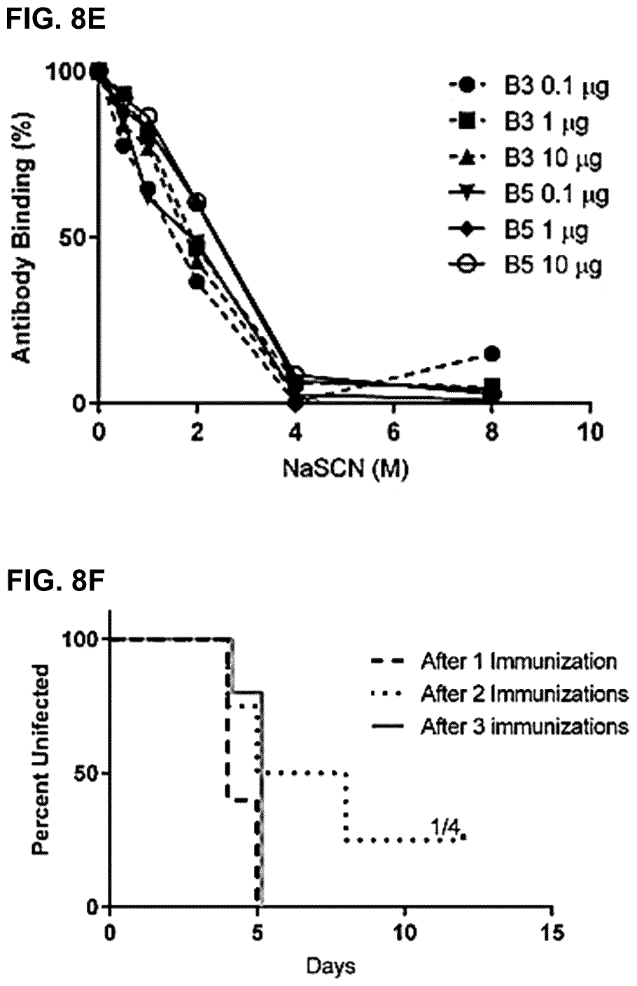

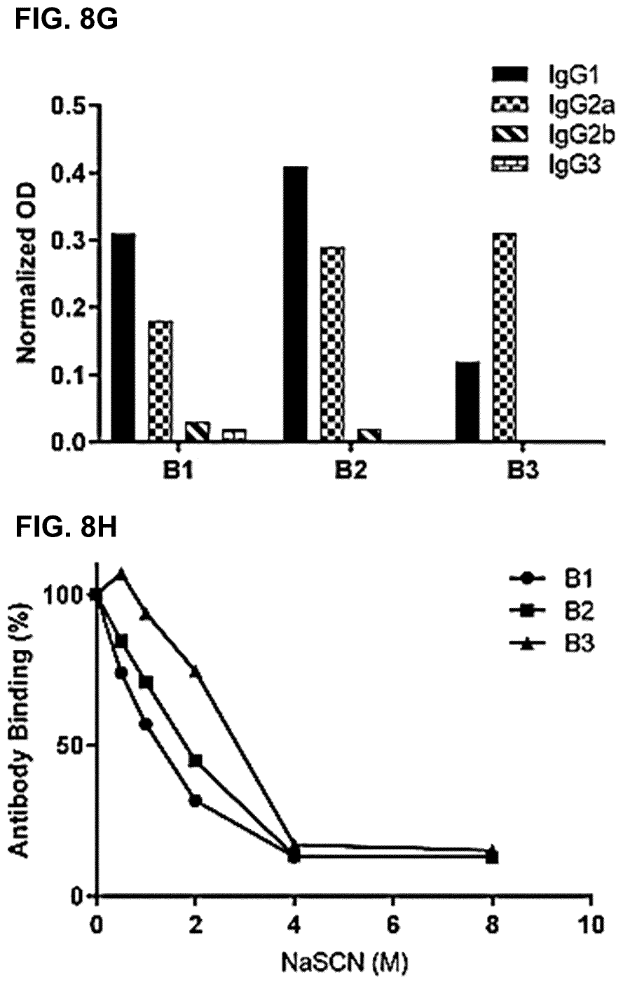

As above, the immunogenicity of the lower doses of the PfCSP mRNA-LNP vaccine was re-examined. Mice were immunized with 0.1 μg, 1 μg, or 10 μg PfCSP mRNA-LNP (schedule in FIG. 2C), followed by an evaluation of antibody responses in bleeds (B3 and B5) and protective efficacy. There was a sharp increase in anti-PfCSP antibody response in bleed B3 from 0.1 μg to 1.0 μg, with geometric mean titers of 218,000, 1, 268,600, and 1,968,300 in mice immunized with three doses of PfCSP mRNA-LNP, respectively. Immunization with another dose did not appreciably boost antibody responses in the bleed B5 in the various groups, except for the 10 μg PfCSP mRNA-LNP group, which showed a ˜50% increase in antibody titers (FIG. 8A).

To assess the protective efficacy of immunization with low doses of PfCSP mRNA-LNP, mice were challenged with sporozoites of PbPfCSP-GFPLuc. Even though significant levels of antibodies were detected, mice in the various dose groups immunized three times were not protected against the challenge infection. Blood-stage parasitemia was detected beginning on day 3 post-challenge in all the mice, however, mice immunized with 1 μg and 10 μg PfCSP mRNA-LNP revealed a one-day difference in the detection of parasites when compared with the Pfs25 mRNA-LNP control group, resulting in significant differences in survival curves between the Pfs25 control group and the 1 μg (p=0.0145) and 10 μg (p=0.0145) PfCSP mRNA-LNP groups (FIG. 8B).

Given these results, the challenged mice were drug cured and immunized one more time, and subsequently challenged as per the schedule in FIG. 2C. As shown in FIG. 8C, 4 out of 5 mice in the 10 μg PfCSP mRNA-LNP group and 3 out of 5 mice in the 1 μg PfCSP mRNA-LNP group showed complete protection over 14 days period of monitoring blood-stage parasitemia. However, mice immunized with the 0.1 μg PfCSP mRNA-LNP were not protected against the challenge infection. The survival curves of uninfected mice of the 1 μg PfCSP mRNA-LNP group (p=0.0031) and the 10 μg PfCSP mRNA-LNP group (p=0.0031) were statistically different compared to the Pfs25 mRNA-LNP control group (FIG. 8C). There was no evidence that the survival curve of the 0.1 μg group was different from the Pfs25 mRNA-LNP negative control group (p=0.0951). Results in FIG. 8D present evidence for a shift in antibody isotype profile from a balanced isotype response in the mice immunized with 0.1 μg and 1.0 μg dose which becomes IgG2a skewed in mice immunized with the higher 10 μg dose. Apart from this isotype profile shift, no significant difference was observed in the avidity index among the 0.1 μg, 1 μg and 10 μg PfCSP mRNA-LNP groups with indices of 1.72, 2.33 and 2.40. The avidity indices of sera collected prior to any challenge were 1.52, 1.91 and 1.80 (FIG. 8E) and the difference between bleeds are within the experimental variability of the assay, thus ruling out any modulation as a result of the exposure to the transgenic parasites used in the first challenge.

During these studies, the immunogenicity parameters and functional protective efficacy in mice immunized were evaluated with different immunization regimens (1, 2, and 3 immunizations) of 10 μg of Pfs25 or PfCSP mRNA-LNPs (FIG. 2A-FIG. 2C). FIG. 7B shows the results of transmission-blocking SMFA in the presence of purified IgG from mice immunized with 10 μg Pfs25 I′-LNP. IgG from sera after a single immunization (FB1) revealed only modest TRA. However, IgG from mice immunized two (FB2) or three times (B4) exhibited robust TRA. The FIG. 7B inset also shows data on antibody avidity and isotype for each bleed with results similar to those in earlier studies (FIG. 5). Results of protection against sporozoite challenge in groups of mice immunized with various regimens of 10 μg PfCSP mRNA-LNP are shown in FIG. 8F. Consistent with previous results showing the importance of a four-dose regimen (FIG. 6 and FIG. 8C), one, two, or three immunizations were not sufficient in giving any significant protection against sporozoite challenge (FIG. 8F). Further analysis of immune sera did reveal an antigen-specific effect of improved avidity with subsequent immunization. The pooled sera was tested after each immunization and then reconfirmed by testing individual mouse serum. As shown in FIG. 8H, the mean avidity indices increased from 0.908 to 1.25, and 1.886 in mice receiving one, two, or three immunizations of 10 μg PfCSP mRNA-LNP, respectively. A significant difference was detected between the mice that received one immunization and mice that received three immunizations (p=0.0090). The isotype analysis revealed once again IgG2a skewed antibodies after three immunizations (FIG. 8G).

### Example 8

Analysis of Splenic T Cell and B Cell Responses in Mice Immunized with Pfs25 and PfCSP mRNA-LNPs

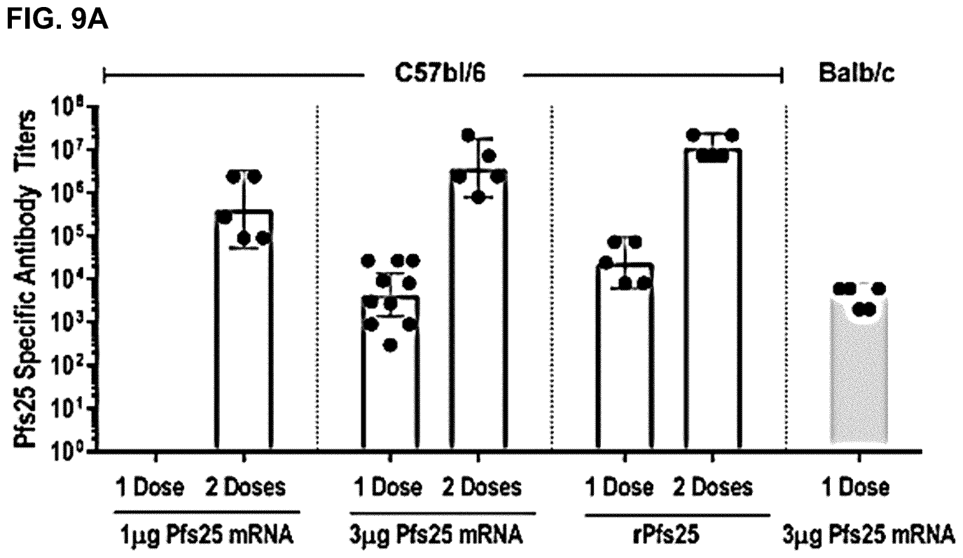

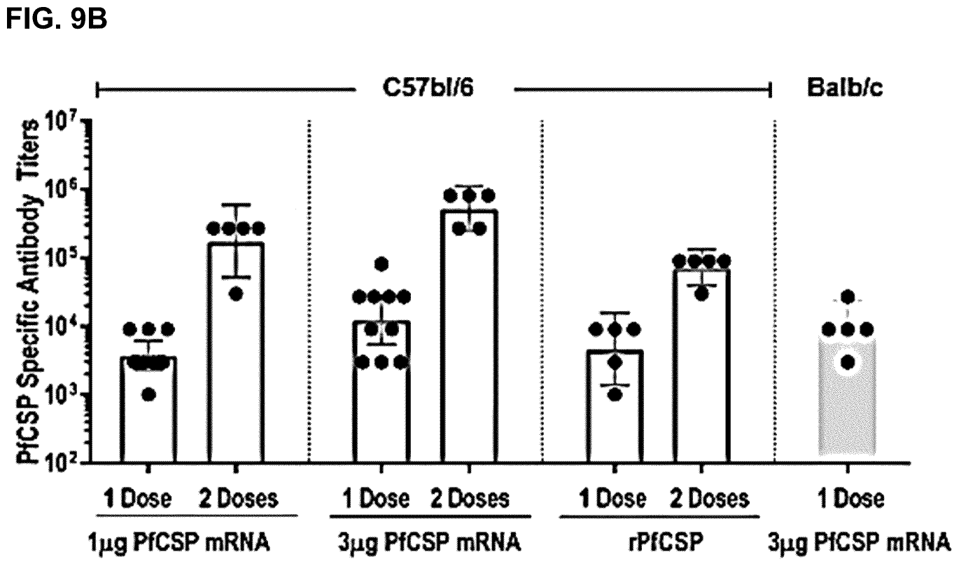

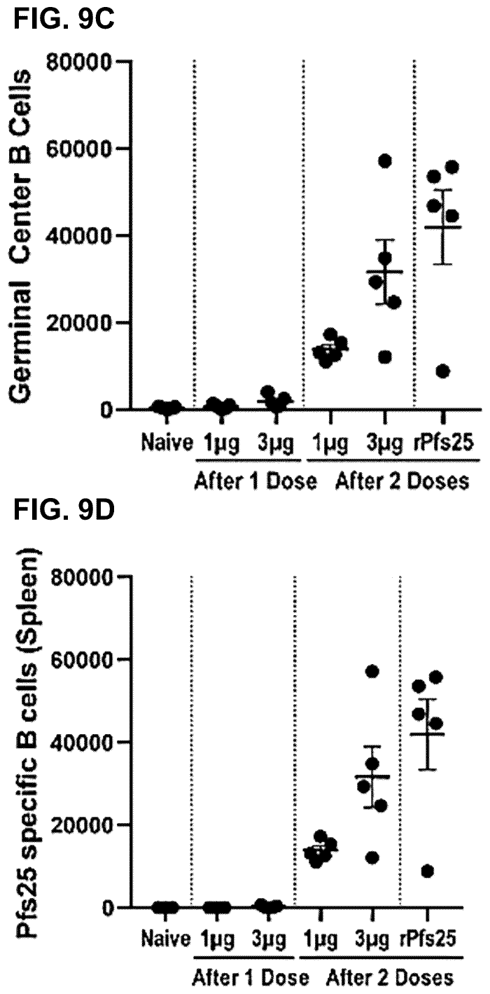

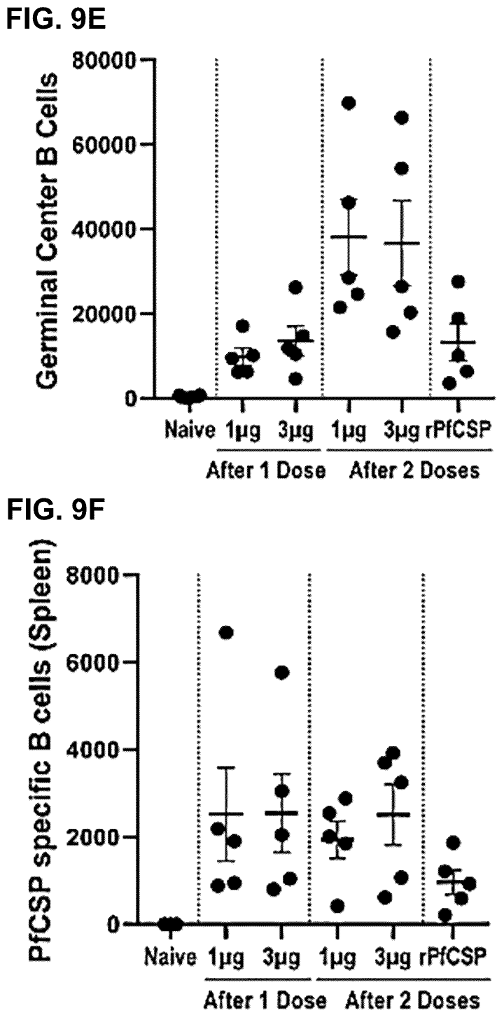

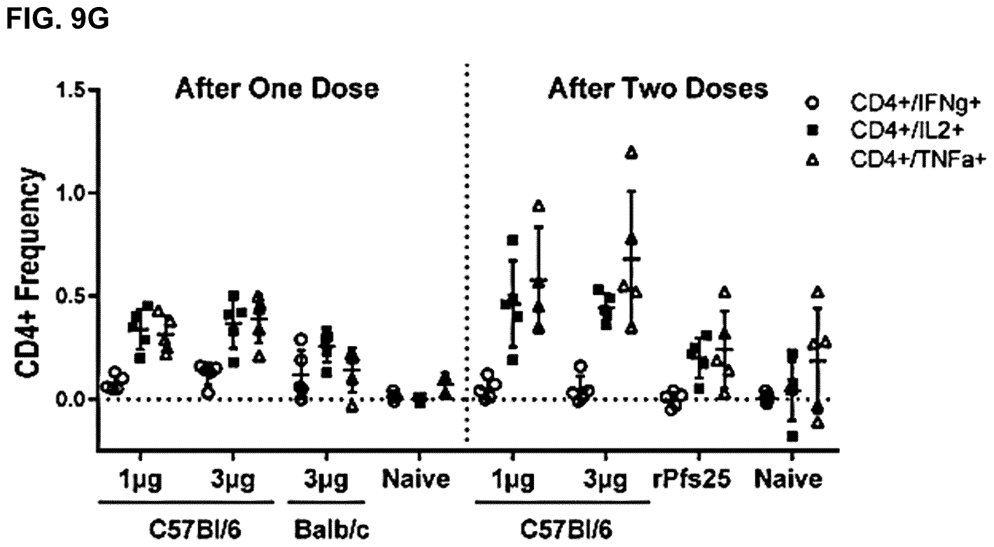

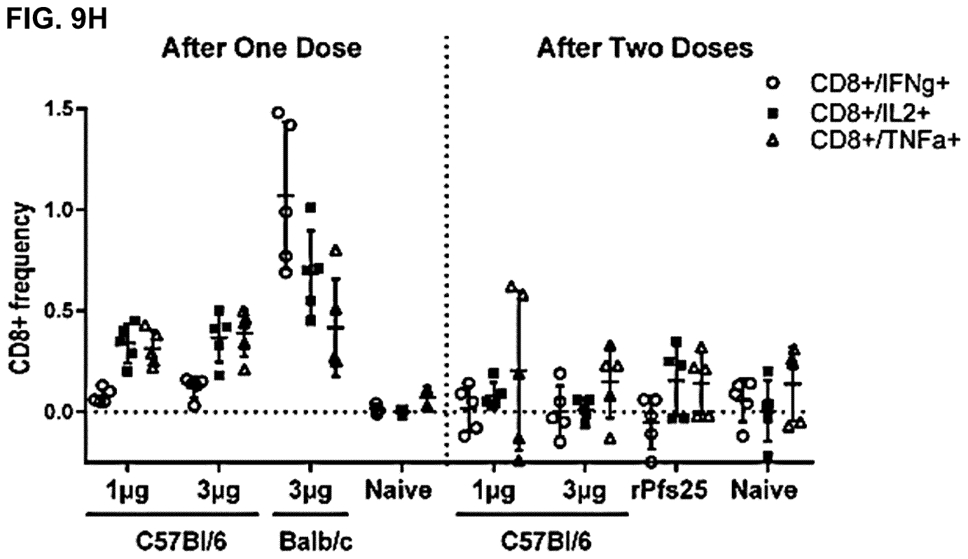

The cellular responses of each mRNA-LNP were investigated by the immunization of female Balb/c and C57Bl/6 mice. The C57Bl/6 mice were immunized with either one or two doses of either Pfs25 or PfCSP mRNA-LNP (1 μg or 3 μg) while the Balb/c mice were immunized with a single 3 μg dose of Pfs25 or PfCSP mRNA-LNP. Additionally, mice immunized with either recombinant Pfs25 or PfCSP proteins administered with the adjuvant, Alhydrogel, were used as a positive control. To confirm the immunogenicity of the immunization, antigen-specific antibody responses were determined from sera collected two weeks following each immunization (FIG. 9A-FIG. 9B). The C57Bl/6 mice immunized with 1 μg of Pfs25 mRNA-LNP were unresponsive two weeks following the first immunization while the C57Bl/6 mice immunized with 3 μg Pfs25 mRNA-LNP elicited antibody titers comparable to antibody responses of Balb/c mice. After a second immunization, antibody titers for all groups increased as expected.

The GC B cells and antigen-specific B cells were measured via flow cytometry of splenocytes collected from the immunized C57Bl/6 mice. Spleens collected from unimmunized mice were used as a control. The number of GC B cells was very low following one immunization and increased significantly following the second dose. In comparison, the amount of GC B cells of mRNA-LNP immunized mice were lower than GC B cells of rPfs25 immunized mice (FIG. 9C). The same trend occurred with Pfs25-specific B cells, with very low number of cells observed after the first immunization and higher amounts observed following the second immunization (FIG. 9D). For PfCSP immunized mice, modest amount of GC B cells was observed in mice immunized with one dose of PfCSP mRNA-LNP and increased significantly following the second dose (FIG. 9E). Two doses of PfCSP mRNA-LNPs elicited higher GC B cells than two doses of rPfCSP with Alhydrogel. Interestingly PfCSP-specific B cells were very low for all groups, yet were similar between all PfCSP mRNA-LNP immunized mice and did not increase following a booster dose (FIG. 9F). Additionally, all PfCSP mRNA-LNP immunized mice had higher PfCSP-specific B cells than those of rPfCSP immunized mice.

For Pfs25 mRNA-LNPs, T cell responses were evaluated. Splenocytes were harvested and cultured with or without stimulation with Pfs25 overlapping peptide pools and the frequency of proliferated splenic T cells producing key cytokines as a result of stimulation was determined. The C57Bl/6 mice immunized with one dose of Pfs25 mRNA-LNP elicited extremely low IL4 and IL5 producing CD4 T cells, while IFNγ, IL2, and TNFα producing CD4 T cells were elevated, showing a Th1 skewed response. C57Bl/6 mice immunized with two doses of Pfs25 mRNA-LNP elicited elevated IL2 and TNFα producing CD4 T cells compared with mice those receiving one dose. In contrast, IFNγ producing CD4 T cells were similar between the one dose and two dose regimens (FIG. 9G). In C57Bl/6 mice, the CD8 T cells producing IFNγ, IL2, and TNFα were elevated in mice that received one dose but were low in mice that received two doses (FIG. 9H).

Additionally, strain-specific T cells responses were observed. The Balb/c mice which received one 3 μg dose had IFNγ producing CD4 T cells that were similar to the C57Bl/6 mice which received one 3 μg dose while IL2 and TNFα producing CD4 T cells were slightly lower (FIG. 9G). In contrast, IFNγ and IL2 producing CD8 T cells were significantly higher in Balb/c mice compared with C57Bl/6 mice (FIG. 9H). Thus, a clear difference in T cell responses was observed between the two mouse strains.

### Summary of Experiments

Malaria vaccine development has focused on antigens expressed during various stages of the life cycle of the parasite. Malaria transmission depends upon the development of intraerythrocytic sexual stages, ingestion by female anopheline mosquitoes, and subsequent sexual development in mosquitoes leading to the formation of sporozoites. An infected Anopheles mosquito initiates the malaria infection cycle by injecting sporozoites into the host which invade hepatocytes leading to pathogenic blood-stage infection. Hence immune interventions aimed at blocking the development of both the liver stage and sexual stage are expected to provide a more effective strategy to protect against malaria infection and transmission. A transmission-blocking vaccine (TBV) approach targeting antigens in the sexual stages (i.e., male and female gametocytes and gametes) and the mosquito stages of the parasite (i.e., zygote and ookinete) is believed to be of central importance in malaria elimination efforts.

Accordingly, the primary goal of the studies reported herein was to evaluate the mRNA-LNP platform for the development of vaccines targeting multiple vulnerable life cycle stages of the malaria parasite. The studies focused on immunogenicity and functional (protective) activity of immune responses elicited by mRNA-LNP encoding the two P. falciparum target antigens; PfCSP (circumsporozoite protein present on the surface of sporozoites initiating infection) and Pfs25 (a target antigen for the development of a TBV).

The data presented herein show that Pfs25 mRNA-LNPs were highly immunogenic at the various doses evaluated. Doses ranging from 0.1 μg to 30 μg elicited strong antibody responses when given in a 3-dose immunization regimen, spaced 4 weeks apart. Antibody responses tended to be modest following the priming dose, which then increased significantly following a booster dose. The ability to effectively prime immune responses was also supported by the quantification of germinal center B cells. Following a single dose, a low level of germinal center B cells was observed in the spleen. This increased significantly after a booster dose. Additionally, robust CD4 and CD8 T cell responses were observed in both C57Bl/6 and Balb/c mice. Strain-specific responses were observed, where Balb/c mice had a significantly stronger CD8 T cell response compared with C57Bl/6 mice.

For all doses of Pfs25 mRNA-LNP given in a three-immunization regimen, the SMFAs revealed potent TRA and TBA in the presence of low IgG concentrations. Even vaccine doses as low as 0.1 μg Pfs25 mRNA-LNPs elicited significant TRA, which improved further as higher vaccine doses were tested. A low dose of 1 μg appeared to optimally provide both high immunogenicity and potent functional mosquito transmission-blocking activity. Immunization with 1 μg and higher amounts of Pfs25 mRNA-LNP revealed highly significant transmission-blocking activity with greater than 90% transmission-blocking activity by IgG concentration of 0.125 mg/ml. Additionally, after three immunizations, the 10 μg Pfs25 mRNA-LNPs elicited ˜2.5 greater antibody titers compared with the 1 μg Pfs25 mRNA-LNPs; however, both their respective TRA and TBA functional activities were comparable.

Additional studies looking at optimization of immunogenicity revealed that even though antibodies elicited by a single dose of a higher amount (10 μg) of mRNA-LNPs elicited transmission reducing antibodies, a booster immunization generated potent functional activity. It is known that modified mRNA-LNP vaccines induce potent Tfh cell responses that drive potent GC responses. Modified mRNA-LNP induced Tfh cells also drive affinity maturation and responses to subdominant epitopes (Pardi N, et al. (2018) J Exp Med. 215(6):1571-1588; Alameh M G, et al. (2021) Immunity. 54(12):2877-2892.e7). Interestingly, mice immunized with the highest dose (30 μg) revealed superior antibody titers as compared to the 3 μg and 10 μg Pfs25 mRNA-LNP groups; however, the antibodies appeared to be less effective in TRA. Seeking an understanding of the impact of parasite challenge through the analysis of antibody avidity and antibody isotypes (IgG1/IgG2a ratio), it was determined that they were not significantly modulated by parasite challenge. The parasite challenge can modulate the humoral response by eliciting antibodies of other specificities, which in turn, can lead to a reduction in the relative proportions of Pfs25-specific antibodies in the purified IgG, reflected in reduced TRA activity in the SMFA.

As with Pfs25 mRNA-LNP, PfCSP mRNA LNP elicited moderate antibody responses following one immunization, and robust responses after each subsequent booster dose. Although antibody titers were high, immunized mice were not completely protected against sporozoite challenge following three immunizations. A significant 1-day delay in the blood-stage parasitemia in the 1 μg and 10 μg PfCSP mRNA-LNP vaccine groups was observed when compared with the negative control group. This indicated that some level of protection was conferred. A one-day delay, however, corresponded to a ˜90% reduction in liver stage burden. Then, mice immunized with PfCSP mRNA-LNPs were given another booster immunization and sporozoite challenge to observe whether another immunization would improve protection. Interestingly, mice immunized with four doses revealed a significant percentage of mice eliciting complete protection.

Additionally, the studies described herein indicated that PfCSP mRNA-LNP immunogenicity requires greater than three immunizations, as has been observed in humans with RTS,S/AS01 (Casares S, et al. (2010) The RTS,S malaria vaccine. Vaccine 28:4880-4894). This can provide a rationale for a heterologous boosting strategy used for PfCSP vaccine evaluation (Sklar M J, et al. (2021) PLOS One. 16:e0256980; Noe A R, et al. (2014) PLOS One. 9:e107764; Wang R, et al. (2004) J Immunol. 172(9):5561-5569).

Apart from exploring the immunogenicity of the mRNA-LNP vaccines encoding Pfs25 and PfCSP alone, another key goal of the work described herein was to evaluate the possibility of co-immunization of mRNA-LNPs affecting different parasite life cycle stages to effectively perturb propagation of infection and transmission cycles. The co-immunization of Pfs25 mRNA-LNP and PfCSP mRNA-LNPs elicited comparable antigen-specific antibody responses to the single antigen mRNA-LNPs. Both the antibody responses and the functional activities after immunization with single antigen or combination vaccines were found to be comparable thus providing supporting evidence in favor of the possibility of combining multiple vaccines without any negative consequences using the mRNA-LNP platform. Furthermore, although PfCSP mRNA-LNP was not completely protective, the overall efficacy when in combination with Pfs25 mRNA-LNP may be enhanced over multiple generations, as was shown in a multigenerational P. berghei mouse model using a combination of passively transferred partially effective antibodies against P25 and PCSP (Sherrard-Smith E, et al. (2018) Elife 7:e35213).

Overall, the use of the mRNA-LNP platform for targeting the malarial antigens, Pfs25 and PfCSP, was highly effective in eliciting protective immunogenicity outcomes. The Pfs25 mRNA-LNP is extremely promising as both low dose immunizations and short prime-boost regimens elicited extremely potent functional activity. The PfCSP mRNA-LNP can benefit from multiple booster immunization or a modified strategy that incorporates a mRNA prime and a heterologous protein boost to elicit complete protection. While a vaccine targeting sporozoite can prevent or reduce the development of blood-stage parasites including gametocytes in an infected person, a TBV can block the sexual reproduction of the gametocytes in the mosquito. A combination of vaccines targeting both the infection stage and sexual/midgut stages—as thoroughly described herein—can provide effective ways to interrupt malaria transmission, which is critical for achieving elimination goals. Here, for the first time, the protective immunogenicity of two of the leading targets, Pfs25 and PfCSP, as nucleoside-modified mRNA-LNP vaccines was evaluated. The work described herein demonstrated for the first time extremely potent protective outcomes. Moreover, when these two were combined into a single vaccine, the studies demonstrated uncompromised outcome against each vaccine's efficacy, which indicates that the mRNA vaccine can combined to provide additive and/or synergistic vaccine induced protection. The work is significant because the only licensed vaccine “Mosquirix” based on PfCSP domains has shown much less than 40% efficacy and thus underscores the need for more effective vaccines.

1. A messenger RNA (mRNA) molecule, comprising:
one or more coding regions encoding at least one antigenic peptide or protein, wherein the at least one antigenic peptide or protein is present in one or more life cycle stages of a malarial parasite.

2. The mRNA molecule of claim 1, wherein the one or more life cycle stages of the malarial parasite comprises the sporozoite stage, the liver stage, the blood-stage, and the sexual-stage.

3. The mRNA molecule of claim 1, wherein the malarial parasite comprises Plasmodium falciparum (P. falciparum).

4. The mRNA molecule of claim 3, wherein the one or more coding regions encode Plasmodium falciparum surface protein Pfs25 or Plasmodium falciparum surface protein circumsporozoite protein (PfCSP).

5. (canceled)

6. The mRNA molecule of claim 4, wherein the encoded Pfs25 comprises a sequence having at least 90% identity to the sequence set forth in SEQ ID NO:01 or SEQ ID NO:07.

7. (canceled)

8. (canceled)

9. The mRNA molecule of claim 4, wherein the coding region for Pfs25 comprises the sequence set forth in SEQ ID NO:09.

10. (canceled)

11. (canceled)

12. The mRNA molecule of claim 4, wherein the encoded PfCSP comprises a sequence having at least 90% identity to the sequence set forth in SEQ ID NO:02 or SEQ ID NO:08.

13. (canceled)

14. (canceled)

15. The mRNA molecule of claim 4, wherein the coding region for PfCSP comprises the sequence set forth in SEQ ID NO:10.

16. The mRNA molecule of claim 1, wherein the mRNA molecule is encapsulated in a lipid nanoparticle.

17. An mRNA vaccine, comprising: the mRNA molecule of claim 16.

18. An mRNA vaccine, comprising: the messenger RNA (mRNA) molecule of claim 4, wherein the mRNA molecule encodes Pfs25, and wherein the mRNA molecule is encapsulated in a lipid nanoparticle.

19. An mRNA vaccine, comprising: the messenger RNA (mRNA) molecule of claim 4, wherein the mRNA molecule encodes PfCSP, and wherein the mRNA molecule is encapsulated in a lipid nanoparticle.

20. A method of inducing an antigen specific immune response, the method comprising:
administering to a subject in need thereof the mRNA vaccine of claim 18 in an amount effective to produce an immune response specific to Pfs25; and
administering to the subject the mRNA vaccine of claim 19 in an amount effective to produce an immune response specific to Pfs25.

21. The method of claim 20,
wherein, following the administering steps, the sexual life cycle of the malarial parasite is disrupted.

22. The method of claim 20,
wherein, following the administering steps, the malarial disease progression is slowed.

23. The method of claim 20, wherein the subject has been diagnosed with malaria or is at risk of developing malaria.

24. (canceled)

25. (canceled)

26. The method of claim 20, further comprising repeating one or more times (i) the administering of the mRNA vaccine of claim 18, and (ii) the administering of the mRNA vaccine of claim 19.

27. The method of claim 20, further comprising monitoring the subject for adverse effects.

28. The method of claim 27, wherein in the absence of adverse effects, the method further comprises continuing to treat the subject and/or continuing to monitor the subject, or wherein in the presence of adverse effects, the method further comprises modifying one or more steps of the method.

29. (canceled)

30. The method of claim 28, wherein modifying comprises modifying the treating step, modifying the administering step, or both.
